(19) United States
(12) Patent Application Publication
(10) Pub. No.: US 2025/0057808 A1
(43) Pub. Date: Feb. 20, 2025

(54) COMPOSITION COMPRISING BETA LACTAM FOR TREATING ALCOHOL DEPENDENCE AND ALCOHOL ASSOCIATED DISEASES OR CONDITIONS

(71) Applicants: Temple University of the Commonwealth System of Higher Education, Philadelphia, PA (US); University of Toledo, Toledo, OH (US)

(72) Inventors: Magid Abou-Gharbia, Exton, PA (US); Wayne Childers, New Hope, PA (US); Youssef Sari, Ottawa Hills, OH (US)

(21) Appl. No.: 18/789,242

(22) Filed: Jul. 30, 2024

Related U.S. Application Data
(60) Provisional application No. 63/516,547, filed on Jul. 31, 2023.

(51) Int. Cl. A61K 31/397; A61K 31/498; A61K 31/695; A61P 25/32

(52) U.S. Cl. CPC A61K 31/397; A61K 31/498; A61K 31/695; A61P 25/32

(57) ABSTRACT

A method is provided for treating or preventing alcohol dependence and/or an alcohol associated disease or condition. Such a method includes administrating a pharmaceutical composition comprising an effective amount of a compound having formula (I):

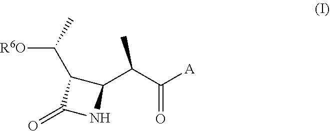

or a hydrates, a solvate, a pharmaceutically acceptable salt, a prodrug, or a complex thereof to a subject in need thereof. For example, (3S, 4R)-3-((R)-(1-hydroxy-ethyl)-4-((R)-[1-methyl-2-(4-methyl-piperazin-1-yl)-2-oxo-ethyl]-azetidin-2-one (MC-100093) is an exemplary compound. The composition can be used to prevent or treat alcohol dependence, attenuate alteration in glutamate transporters, reduce alcohol associated neuroinflammation, and treat or prevent alcohol associated fatty liver diseases.

### PRIORITY CLAIM AND CROSS-REFERENCE

This application claims the benefit of U.S. Provisional Application No. 63/516,547, filed Jul. 31, 2023, which application is expressly incorporated by reference herein in its entirety.

### STATEMENT REGARDING FEDERALLY SPONSORED RESEARCH OR DEVELOPMENT

This invention was made with government support under R01 AA029674 and R01 DA047270 awarded by the National Institutes of Health (NIH). The government has certain rights in the invention.

### FIELD

The disclosure relates to a pharmaceutical composition and a method of use generally. More particularly, the disclosed subject matter relates to pharmaceutical compositions and methods for treating alcohol dependence and alcohol-associated diseases such as fatty liver diseases.

### BACKGROUND

Alcohol use disorder (AUD) is a complex chronically relapsing disorder that, as it progresses, induces long-lasting neuroadaptations as a result of alterations in brain circuitry. Among these adaptations, glutamatergic changes have been implicated in the development and maintenance of ethanol dependence. Dysregulation of the glutamatergic systems in key brain reward regions such as nucleus accumbens (NAc), basal lateral amygdala (BLA), prefrontal cortex (PFC), and hippocampus is an observed consequence of chronic ethanol exposure, resulting in altered glutamate receptors and transporters. Among other brain regions, the NAc is an important reward region that has been extensively studied for its crucial role in the development of substance use disorders (SUDs), including ethanol.

In addition to brain effects, alcohol exposure causes chronic liver injury, particularly steatohepatitis. Peroxisome proliferator-activated receptors (PPAR-α and PPARγ) play critical roles in adipose expansion and in the control of their function. Studies linked the ethanol induced fatty liver to the blocking of PPAR-α activity in vitro and in vivo. Moreover, PPAR agonists reduced ethanol drinking behavior, neurodegeneration, and alcohol induced liver injury. It is noteworthy that ethanol, in addition to its direct action on the brain, may impair neurotransmitter function required for some aspects of neuroplasticity, learning, and memory through the liver-brain axis, including via the development of insulin resistance. Accordingly, these studies suggested the role of liver-brain axis in the ethanol dependence.

### SUMMARY

The present disclosure provides compounds, compositions, and methods for treating, lessening effect of, or preventing alcohol dependence and alcohol-associated fatty liver diseases. The compounds and the compositions comprise at least one beta lactam.

In one aspect, the present disclosure provides a composition comprising a compound having formula (I)

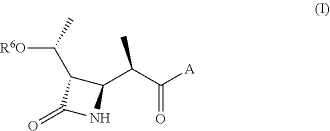

including hydrates, solvates, pharmaceutically acceptable salts, prodrugs and complexes thereof, wherein:
A is selected from the group consisting of

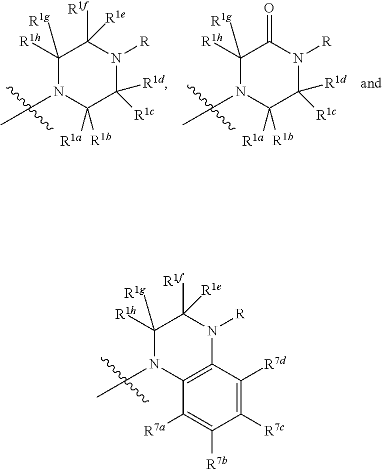

R is selected from the group consisting of hydrogen, C₁₋₆ linear alkyl, C₁₋₆ branched alkyl, optionally substituted aryl, C(O)R², C(O)OR³, C(O)NR^{4a}R^{4b}, SO₂R⁵, and SO₂NH₂;
R^{1a}, R^{1b}, R^{1e}, R^{1d}, R^{1e}, R^{1f}, R^{1g}, and R^{1h} are each independently selected from the group consisting of hydrogen, C₁₋₆ linear alkyl, and C₁₋₆ branch alkyl;
In some embodiments, R^{1b} and R^{1g} are joined together with the atoms to which they are bound to form a ring containing 5, 6, or 7 atoms;
In some embodiments, R^{1b} and R^{1f} are joined together with the atoms to which they are bound to form a ring containing 5, 6, or 7 atoms;
In some embodiments, R^{1d} and R^{1f} are joined together with the atoms to which they are bound to form a ring containing 5, 6, or 7 atoms;
In some embodiments, R^{1b} and R^{1c} are joined together with the atoms to which they are bound to form a ring containing 5 or 6 atoms;
R² is selected from the group consisting of C₁₋₆ linear alkyl, C₁₋₆ branched alkyl, and optionally substituted aryl;
R³ is selected from the group consisting of C₁₋₆ linear alkyl, C₁₋₆ branched alkyl, and optionally substituted aryl;
R^{4a} is selected from the group consisting of C₁₋₆ linear alkyl, C₁₋₆ branched alkyl, and optionally substituted aryl;
R^{4b} is selected from the group consisting of C₁₋₆ linear alkyl, C₁₋₆ branched alkyl, and optionally substituted aryl;
R⁵ is selected from the group consisting of C₁₋₆ linear alkyl, C₁₋₆ branched alkyl, and optionally substituted aryl;
R⁶ is selected from the group consisting of hydrogen, C₁₋₆ linear alkyl, and C(O)R⁸;
R^{7a}, R^{7b}, R^{7c}, and R^{7d} are each independently selected from the group consisting of are each independently selected from a group consisting of hydrogen, halogen, OH, C₁₋₆ linear alkyl, C₁₋₆ branched alkyl, C₁₋₆ alkoxy, C₁₋₆ haloalkyl, C₁₋₆ haloalkoxy, cyano, NH(C₁₋₆ alkyl), N(C₁₋₆ alkyl)₂, NHC(O)R⁸, C(O)NHR⁸, C(O)N(R⁸)₂, SH, SC₁₋₆ alkyl, SO₂NH₂, SO₂NHR⁸, SO₂R⁸, and NHSO₂R⁸;
R⁸ is independently selected at each occurrence from a group consisting of hydrogen, C₁₋₆ linear alkyl, C₁₋₆ branched alkyl, and C₃₋₇ cycloalkyl;
The present disclosure further provides compositions comprising:
   an effective amount of one or more compounds according to the present disclosure and at least one excipient.

The present disclosure also provides a method for preventing, treating, or lessen effects of, alcohol dependence. Such as method comprises administering to a subject an effective amount of a compound or composition according to the present disclosure. Such a method includes administering to a subject in need thereof a composition comprising an effective amount of one or more compounds according to the present disclosure and at least one excipient.

In some embodiments, the alcohol dependence is treated or prevented by attenuating alteration in glutamate transporters and/or reducing alcohol associated neuroinflammation.

The present disclosure also provides a method for preventing, treating, or lessen effects of alteration in glutamate transporters in the subject associated with alcohol uses. In some embodiments, the method is for attenuating alteration in glutamate transporters in the subject. Such as method comprises administering to a subject an effective amount of a compound or composition according to the present disclosure. Such a method includes administering to a subject in need thereof a composition comprising an effective amount of one or more compounds according to the present disclosure and at least one excipient.

The present disclosure also provides a method for reducing alcohol associated neuroinflammation in the subject. Such as method comprises administering to a subject an effective amount of a compound or composition according to the present disclosure. Such a method includes administering to a subject in need thereof a composition comprising an effective amount of one or more compounds according to the present disclosure and at least one excipient.

The present disclosure also provides a method for preventing, treating, or lessen effects of, alcohol-associated fatty liver diseases. Such as method comprises administering to a subject an effective amount of a compound or composition according to the present disclosure. Such a method includes administering to a subject in need thereof a composition comprising an effective amount of one or more compounds according to the present disclosure and at least one excipient.

In some embodiments, the composition is administrated orally or through injection. The effective amount is a dose of the compound may be in a range of from 0.001 mg/Kg of the subject to 200 mg/Kg of the subject. For example, the dose of the compound is in a range of from 1 mg/Kg of the subject to 100 mg/Kg of the subject.

In the methods described herein, in some embodiments, the compound is (3S, 4R)-3-((R)-(1-hydroxy-ethyl)-4-((R)-[1-methyl-2-(4-methyl-piperazin-1-yl)-2-oxo-ethyl]-azetidin-2-one (MC-100093), or a pharmaceutically acceptable salt or complex thereof. The pharmaceutical composition further comprises at least one excipient. The composition is administrated through injection in some embodiments.

The present disclosure further provides a process for preparing the compounds and the compositions of the present disclosure.

### BRIEF DESCRIPTION OF THE DRAWINGS

The present disclosure is best understood from the following detailed description when read in conjunction with the accompanying drawings. It is emphasized that, according to common practice, the various features of the drawings are not necessarily to scale. On the contrary, the dimensions of the various features are arbitrarily expanded or reduced for clarity. Like reference numerals denote like features throughout specification and drawings.

FIGS. 1A-1C show the effects of daily MC-100093 (50 mg/kg, i.p.) or ceftriaxone (200 mg/kg, i.p.) treatment for five consecutive days on (A) Ethanol consumption (g/kg of average body weight/24 hr), (B) water intake (mL/day), and (C) body weight (grams). MC-100093 is (3S, 4R)-3-((R)-(1-hydroxy-ethyl)-4-((R)-[1-methyl-2-(4-methyl-piperazin-1-yl)-2-oxo-ethyl]-azetidin-2-one as an exemplary compound provided in the present disclosure.

FIGS. 2A-2D show the effects of MC-100093 (50 mg/kg, i.p.) or ceftriaxone (200 mg/kg, i.p.) treatment on: (A) Protein expression of GLT-1 in the NAc-core, (B) Protein expression of GLT-1 in the NAc-shell, (C) Protein expression of xCT in the NAc-core, and (D) Protein expression of xCT in the NAc-shell.

FIGS. 3A-3F show the effects of MC-100093 (50 mg/kg, i.p.) or ceftriaxone (200 mg/kg, i.p.) treatment on: (A) Protein expression of mGluR1 in the NAc-core, (B) Protein expression of mGluR1 in the NAc-shell, (C) Protein expression of mGluR5 in the NAc-core, (D) Protein expression of mGluR5 in the NAc-shell, (E) Protein expression of PPAR-α in the NAc-core, and (F) Protein expression of PPAR-α in the NAc-shell.

FIGS. 4A-4B show fat droplets in Oil Red O stained liver sections: (A) Quantitative analysis in liver fat content (high fat deposition) in ethanol group compared to control group, and (B) Representative Oil Red O stained liver sections of control, ethanol, ethanol-MC and ethanol-CEF rats.

FIGS. 5A-5D show the effects of MC-100093 (50 mg/kg, i.p.) or ceftriaxone (200 mg/kg, i.p.) on: (A) Protein expression of PPAR-α in the liver, (B) Protein expression of PPAR-γ in the liver, (C) Protein expression of GLT-1 in the liver, and (D) Protein expression of xCT in the liver.

FIGS. 6A-6B show the effects of five consecutive days of MC-100093 (100 mg/kg, i.p.) or saline (i.p.) treatment on (A) ethanol consumption (g/kg/24 hr), and (B) water intake (mL/day).

FIG. 7 shows the effect of five consecutive days of MC-100093 (100 mg/kg, i.p.) or saline (i.p.) treatment on body weight (grams).

FIGS. 8A-8B show the effects of five consecutive days of MC-100093 (100 mg/kg, i.p.) or saline (i.p.) on GLT-1 expression in the infralimbic prefrontal cortex (IL).

FIGS. 9A-9B show the effects of five consecutive days of MC-100093 (100 mg/kg, i.p.) or saline (i.p.) on GLT-1 expression in the prelimbic frontal cortex (PL).

FIGS. 10A-10D show the effects of MC-100093 treatment (100 mg/kg, i.p.) in male P rats for five consecutive days (A) Ethanol consumption (g/kg of average body weight/24 hr), (B) Ethanol preference, (C) Average water intake (mL/day), and (C) body weight (grams). Values are expressed as mean±SEM (n=5-7/group), (*p<0.05 and **p<0.01, ***p<0.001, and ****p<0.0001).

FIGS. 11A-11D show the effects of MC-100093 treatment (100 mg/kg, i.p.) in female P rats for five consecutive days (A) Ethanol consumption (g/kg of average body weight/24 hr), (B) Ethanol preference, (C) Average water intake (mL/day), and (C) body weight (grams). Values are expressed as mean±SEM (n=5-7/group), (*p<0.05 and **p<0.01, ***p<0.001, and ****p<0.0001).

FIGS. 12A-12D show the effects of MC-100093 treatment (100 mg/kg, i.p.) for five days on the expression of GLT-1 and xCT expression in the mPFC-IL and mPFC-PL in Male P rats: (A) Immunoblot of GLT-1 and β-tubulin in the mPFC-IL; (B) Immunoblot of xCT and β-tubulin in the mPFC-IL, (C) Immunoblot of GLT-1 and β-tubulin in the mPFC-PL. Quantitative analysis using one-way ANOVA followed by Newman-Keuls test indicated that there was a significant decrease in GLT-1 expression in the ethanol-saline group as compared to water-saline group, while post-treatment with MC-100093 significantly upregulated GLT-1 expression as compared to ethanol-saline group in the mPFC-PL, and (D) Immunoblot of xCT and β-tubulin in the mPFC-PL. Water-saline group data were represented as 100% (relative to water-saline). Values are expressed as mean±SEM (n=5/group), (*p<0.05, **p<0.01, ***p<0.001, and p<0.0001).

FIGS. 13A-13D show the effects of MC-100093 treatment (100 mg/kg, i.p.) for five days on the expression of GLT-1 and xCT in the mPFC-IL and mPFC-PL in Female P rats: (A) Immunoblot of GLT-1 and β-tubulin in the mPFC-IL, (B) Immunoblot of xCT and β-tubulin in the mPFC-IL, (C) Immunoblot of GLT-1 and β-tubulin in the mPFC-PL, and (D) Immunoblot of xCT and β-tubulin in the mPFC-PL. Water-saline group data were represented as 100% (relative to water-saline). Values are expressed as mean±SEM (n=5/group), (*p<0.05, **p<0.01, and ***p<0.001).

FIGS. 14A-14D show the effects of MC-100093 treatment (100 mg/kg, i.p.) for five days on the expression of GLT-1 and xCT in the NAc-shell and NAc-core in Male P rats: (A) Immunoblot of GLT-1 and β-tubulin in the NAc-shell, (B) Immunoblot of xCT and 3-tubulin in the NAc-shell, (C) Immunoblot of GLT-1 and β-tubulin in the NAc-core, and (D) Immunoblot of xCT and β-tubulin in the NAc-core. Water-saline group data were represented as 100% (relative to water-saline). Values are expressed as mean±SEM (n=5/group), (*p<0.05, **p<0.01, and ***p<0.001).

FIGS. 15A-15D show the effects of MC-100093 treatment (100 mg/kg, i.p.) for five days on the expression of GLT-1 and xCT in the NAc-shell and NAc-core in Female P rats: (A) Immunoblot of GLT-1 and β-tubulin in the NAc-shell, (B) Immunoblot of xCT and β-tubulin in the NAc-shell, (C) Immunoblot of GLT-1 and β-tubulin in the NAc-core, and (D) Immunoblot of xCT and β-tubulin in the NAc-core. Water-saline group data were represented as 100% (relative to water-saline). Values are expressed as mean±SEM (n=5/group), (*p<0.05, **p<0.01, and ***p<0.001).

FIG. 16 shows the timeline of experimental procedures for evaluating the effect of MC-100093 on neuroinflammatory and neurotrophic biomarkers in mesocorticolimbic brain regions of make alcohol preferring rates exposed chronically to ethanol. There were three groups: water/saline, ethanol/saline and ethanol/MC-100093 while Ethanol concentrations were 15% and 30%, v/v.

FIGS. 17A-17C show the effect of MC-100093 (100 mg/kg i.p.) on (A) ethanol intake, (B) water intake, and (C) body weight when the testing was performed using the timeline and the procedures in FIG. 16. Each column is expressed as mean±S.E.M (Water/Saline, n=5; Ethanol/Saline, n=5; Ethanol/MC-100093, n=6), (*p<0.05, **p<0.01, and ***p<0.001).

FIGS. 18A-18D show the effects of MC-100093 and chronic ethanol intake on Brain Derived Neurotrophic Factor (BDNF) expression in the subregions of mPFC and nucleus accumbens (NAc). FIGS. 18A-18B show the BDNF expression in the infralimbic (IL) cortex and in the prelimbic (PL) cortex, respectively. FIGS. 18C-18D show the BDNF expression in the nucleus accumbens (NAc) core and the NAc shell, respectively. Control group data were represented as 100%. Each column is expressed as mean±S.E.M, (n=5 per group), (*p<0.05, **p<0.01, ***p<0.001 and ****p<0.0001).

FIGS. 19A-19D show the effects of chronic ethanol intake and MC-100093 on Tumor Necrosis Factor alpha (TNF-α) expression in the subregions of mPFC (IL and PL) and NAc (shell and core). Quantitative analysis using one-way ANOVA followed by Newman Keuls post-hoc test indicated a significant increase in TNF-α expression in the IL (A), PL (B), NAc core (C), and NAc shell (D) in ethanol/saline group as compared to water/control group, while post-treatment with MC-100093 normalized TNF-α expression in all these brain regions. Control group data were represented as 100%. Each column is expressed as mean±S.E.M, (n=5 per group), (*p<0.05, **p<0.01, ***p<0.001 and ****p<0.0001).

FIGS. 20A-20D show the effects of chronic ethanol intake and MC-100093 on Receptor for Advanced Glycation End Products (RAGE) expression in the subregions of mPFC (IL and PL in FIG. 20A and FIG. 20B, respectively) and NAc (shell and core in FIG. 21C and FIG. 21D, respectively). In FIGS. 20B-20D, comparison was made while post-treatment with MC-100093 normalized RAGE expression in corresponding regions. Control group data were represented as 100%. Each column is expressed as mean±S.E.M (n=5 per group), (*p<0.05, **p<0.01, and ****p<0.0001).

FIGS. 21A-21D show the effects of chronic ethanol intake and MC-100093 on High Mobility Group Box 1 (HMGβ-1) expression in the subregions of the mPFC and NAc. Quantitative analysis using one-way ANOVA followed by Newman Keuls post-hoc test revealed an upregulation of HMGβ-1 in the IL (A), PL (B), NAc core (C), and NAc shell (D) in the ethanol/saline group as compared to the water/saline group, while post-treatment with MC-100093 normalized HMGβ-1 expression in all these brain regions. Control group data were represented as 100%. Each column is expressed as mean±S.E.M (n=5 per group), (*p<0.05, **p<0.01, and ***p<0.001).

### DETAILED DESCRIPTION

For purposes of the description hereinafter, it is to be understood that the embodiments described below may assume alternative variations and embodiments. It is also to be understood that the specific articles, compositions, and/or processes described herein are exemplary and should not be considered as limiting.

These and other objects, features, and advantages will become apparent to those of ordinary skill in the art from a reading of the following detailed description and the appended claims. All percentages, ratios and proportions herein are by weight, unless otherwise specified. All temperatures are in degrees Celsius (° C.) unless otherwise specified. All documents cited are in relevant part, incorporated herein by reference; the citation of any document is not to be construed as an admission that it is prior art with respect to the present disclosure.

In the present disclosure the singular forms “a,” “an,” and “the” include the plural reference, and reference to a particular numerical value includes at least that particular value, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

When values are expressed as approximations, by use of the antecedent “about,” it will be understood that the particular value forms another embodiment. As used herein, “about X” (where X is a numerical value) preferably refers to +10% of the recited value, inclusive. For example, the phrase “about 8” preferably refers to a value of 7.2 to 8.8, inclusive; as another example, the phrase “about 8%” preferably (but not always) refers to a value of 7.2% to 8.8%, inclusive. Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, “2-5”, and the like. In addition, when a list of alternatives is positively provided, such listing can be interpreted to mean that any of the alternatives may be excluded, e.g., by a negative limitation in the claims. For example, when a range of “1 to 5” is recited, the recited range may be construed as including situations whereby any of 1, 2, 3, 4, or 5 are negatively excluded; thus, a recitation of “1 to 5” may be construed as “1 and 3-5, but not 2”, or simply “wherein 2 is not included.” It is intended that any component, element, attribute, or step that is positively recited herein may be explicitly excluded in the claims, whether such components, elements, attributes, or steps are listed as alternatives or whether they are recited in isolation.

Throughout the description, where compositions are described as having, including, or comprising specific components, or where processes are described as having, including, or comprising specific process steps, it is contemplated that compositions of the present disclosure also consist essentially of, or consist of, the recited components, and that the processes of the present disclosure also consist essentially of, or consist of, the recited processing steps.

In the application, where an element or component is said to be included in and/or selected from a list of recited elements or components, it should be understood that the element or component can be any one of the recited elements or components and can be selected from a group consisting of two or more of the recited elements or components.

It should be understood that the order of steps or order for performing certain actions is immaterial so long as the present disclosure remain operable. Moreover, two or more steps or actions can be conducted simultaneously.

As used herein, the term “alcohol” is understood to encompass any alcohol (ethanol) containing composition, which includes, but is not limited to, alcoholic beverage or drink. The term “alcohol dependence” refers to a chronic disease, in which a subject such as a human being craves drinks that contain alcohol and is unable to control his or her drinking. The alcohol associated diseases or conditions are understood to encompass a disease or condition associated with excessive consumption of alcohol.

As used herein, the term “halogen” shall mean chlorine, bromine, fluorine and iodine.

As used herein, unless otherwise noted, “alkyl” and “aliphatic” whether used alone or as part of a substituent group refers to straight and branched carbon chains having 1 to 20 carbon atoms or any number within this range, for example 1 to 6 carbon atoms or 1 to 4 carbon atoms. Designated numbers of carbon atoms (e.g. C₁₋₆) shall refer independently to the number of carbon atoms in an alkyl moiety or to the alkyl portion of a larger alkyl-containing substituent. Non-limiting examples of alkyl groups include methyl, ethyl, n-propyl, iso-propyl, n-butyl, sec-butyl, iso-butyl, tert-butyl, and the like. Alkyl groups can be optionally substituted. Non-limiting examples of substituted alkyl groups include hydroxymethyl, chloromethyl, trifluoromethyl, aminomethyl, 1-chloroethyl, 2-hydroxyethyl, 1,2-difluoroethyl, 3-carboxypropyl, and the like. In substituent groups with multiple alkyl groups such as (C₁₋₆alkyl)₂amino, the alkyl groups may be the same or different.

As used herein, the terms “alkenyl” and “alkynyl” groups, whether used alone or as part of a substituent group, refer to straight and branched carbon chains having 2 or more carbon atoms, preferably 2 to 20, wherein an alkenyl chain has at least one double bond in the chain and an alkynyl chain has at least one triple bond in the chain. Alkenyl and alkynyl groups can be optionally substituted. Nonlimiting examples of alkenyl groups include ethenyl, 3-propenyl, 1-propenyl (also 2-methylethenyl), isopropenyl (also 2-methylethen-2-yl), buten-4-yl, and the like. Nonlimiting examples of substituted alkenyl groups include 2-chloroethenyl (also 2-chlorovinyl), 4-hydroxybuten-1-yl, 7-hydroxy-7-methyloct-4-en-2-yl, 7-hydroxy-7-methyloct-3,5-dien-2-yl, and the like. Nonlimiting examples of alkynyl groups include ethynyl, prop-2-ynyl (also propargyl), propyn-1-yl, and 2-methyl-hex-4-yn-1-yl. Nonlimiting examples of substituted alkynyl groups include, 5-hydroxy-5-methylhex-3-ynyl, 6-hydroxy-6-methylhept-3-yn-2-yl, 5-hydroxy-5-ethylhept-3-ynyl, and the like.

As used herein, “cycloalkyl,” whether used alone or as part of another group, refers to a non-aromatic carbon-containing ring including cyclized alkyl, alkenyl, and alkynyl groups, e.g., having from 3 to 14 ring carbon atoms, preferably from 3 to 7 or 3 to 6 ring carbon atoms, or even 3 to 4 ring carbon atoms, and optionally containing one or more (e.g., 1, 2, or 3) double or triple bond. Cycloalkyl groups can be monocyclic (e.g., cyclohexyl) or polycyclic (e.g., containing fused, bridged, and/or spiro ring systems), wherein the carbon atoms are located inside or outside of the ring system. Any suitable ring position of the cycloalkyl group can be covalently linked to the defined chemical structure. Cycloalkyl rings can be optionally substituted. Nonlimiting examples of cycloalkyl groups include: cyclopropyl, 2-methyl-cyclopropyl, cyclopropenyl, cyclobutyl, 2,3-dihydroxycyclobutyl, cyclobutenyl, cyclopentyl, cyclopentenyl, cyclopentadienyl, cyclohexyl, cyclohexenyl, cycloheptyl, cyclooctanyl, decalinyl, 2,5-dimethylcyclopentyl, 3,5-dichlorocyclohexyl, 4-hydroxycyclohexyl, 3,3,5-trimethylcyclohex-1-yl, octahydropentalenyl, octahydro-1H-indenyl, 3a,4,5,6,7,7a-hexahydro-3H-inden-4-yl, decahydroazulenyl; bicyclo[6.2.0]decanyl, decahydronaphthalenyl, and dodecahydro-1H-fluorenyl. The term “cycloalkyl” also includes carbocyclic rings which are bicyclic hydrocarbon rings, non-limiting examples of which include, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

“Haloalkyl” is intended to include both branched and straight-chain saturated aliphatic hydrocarbon groups having the specified number of carbon atoms, substituted with 1 or more halogen. Haloalkyl groups include perhaloalkyl groups, wherein all hydrogens of an alkyl group have been replaced with halogens (e.g., —CF₃, CF₂CF₃). Haloalkyl groups can optionally be substituted with one or more substituents in addition to halogen. Examples of haloalkyl groups include, but are not limited to, fluoromethyl, dichloroethyl, trifluoromethyl, trichloromethyl, pentafluoroethyl, and pentachloroethyl groups.

The term “alkoxy” refers to the group —O-alkyl, wherein the alkyl group is as defined above. Alkoxy groups optionally may be substituted. The term C₃-C₆ cyclic alkoxy refers to a ring containing 3 to 6 carbon atoms and at least one oxygen atom (e.g., tetrahydrofuran, tetrahydro-2H-pyran). C₃-C₆ cyclic alkoxy groups optionally may be substituted.

The term “aryl,” wherein used alone or as part of another group, is defined herein as an unsaturated, aromatic monocyclic ring of 6 carbon members or to an unsaturated, aromatic polycyclic ring of from 10 to 14 carbon members. Aryl rings can be, for example, phenyl or naphthyl ring each optionally substituted with one or more moieties capable of replacing one or more hydrogen atoms. Non-limiting examples of aryl groups include: phenyl, naphthylen-1-yl, naphthylen-2-yl, 4-fluorophenyl, 2-hydroxyphenyl, 3-methylphenyl, 2-amino-4-fluorophenyl, 2-(N,N-diethylamino)phenyl, 2-cyanophenyl, 2,6-di-tert-butylphenyl, 3-methoxyphenyl, 8-hydroxynaphthylen-2-yl 4,5-dimethoxynaphthylen-1-yl, and 6-cyano-naphthylen-1-yl. Aryl groups also include, for example, phenyl or naphthyl rings fused with one or more saturated or partially saturated carbon rings (e.g., bicyclo[4.2.0]octa-1,3,5-trienyl, indanyl), which can be substituted at one or more carbon atoms of the aromatic and/or saturated or partially saturated rings.

The term “arylalkyl” or “aralkyl” refers to the group -alkyl-aryl, where the alkyl and aryl groups are as defined herein. Aralkyl groups of the present disclosure are optionally substituted. Examples of arylalkyl groups include, for example, benzyl, 1-phenylethyl, 1-phenyl-1-methylethyl, 2-phenylethyl, 3-phenylpropyl, 2-phenylpropyl, fluorenylmethyl and the like.

The terms “heterocyclic” and/or “heterocycle” and/or “heterocylyl,” whether used alone or as part of another group, are defined herein as one or more ring having from 3 to 20 atoms wherein at least one atom in at least one ring is a heteroatom selected from nitrogen (N), oxygen (O), or sulfur (S), and wherein further the ring that includes the heteroatom is non-aromatic. In heterocycle groups that include 2 or more fused rings, the non-heteroatom bearing ring may be aryl (e.g., indolinyl, tetrahydroquinolinyl, chromanyl). Exemplary heterocycle groups have from 3 to 14 ring atoms of which from 1 to 5 are heteroatoms independently selected from nitrogen (N), oxygen (O), or sulfur (S). One or more N or S atoms in a heterocycle group can be oxidized. Heterocycle groups can be optionally substituted. Non-limiting examples of heterocyclic units having a single ring include: diazirinyl, aziridinyl, urazolyl, azetidinyl, pyrazolidinyl, imidazolidinyl, oxazolidinyl, isoxazolinyl, isoxazolyl, thiazolidinyl, isothiazolyl, isothiazolinyl oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, piperidin-2-onyl (valerolactam), 2,3,4,5-tetrahydro-1H-azepinyl, 2,3-dihydro-1H-indole, and 1,2,3,4-tetrahydro-quinoline. Non-limiting examples of heterocyclic units having 2 or more rings include: hexahydro-1H-pyrrolizinyl, 3a,4,5,6,7,7a-hexahydro-1H-benzo[d]imidazolyl, 3a,4,5,6,7,7a-hexahydro-1H-indolyl, 1,2,3,4-tetrahydroquinolinyl, chromanyl, isochromanyl, indolinyl, isoindolinyl, and decahydro-1H-cycloocta[b]pyrrolyl.

The term “heteroaryl,” whether used alone or as part of another group, is defined herein as one or more rings having from 5 to 20 atoms wherein at least one atom in at least one ring is a heteroatom chosen from nitrogen (N), oxygen (O), or sulfur (S), and wherein further at least one of the rings that includes a heteroatom is aromatic. In heteroaryl groups that include 2 or more fused rings, the non-heteroatom bearing ring may be a carbocycle (e.g., 6,7-Dihydro-5H-cyclopentapyrimidine) or aryl (e.g., benzofuranyl, benzothiophenyl, indolyl). Exemplary heteroaryl groups have from 5 to 14 ring atoms and contain from 1 to 5 ring heteroatoms independently selected from nitrogen (N), oxygen (O), or sulfur (S). One or more N or S atoms in a heteroaryl group can be oxidized. Heteroaryl groups can be substituted. Non-limiting examples of heteroaryl rings containing a single ring include: 1,2,3,4-tetrazolyl, [1,2,3]triazolyl, [1,2,4]triazolyl, triazinyl, thiazolyl, 1H-imidazolyl, oxazolyl, furanyl, thiopheneyl, pyrimidinyl, 2-phenylpyrimidinyl, pyridinyl, 3-methylpyridinyl, and 4-dimethylaminopyridinyl. Non-limiting examples of heteroaryl rings containing 2 or more fused rings include: benzofuranyl, benzothiophenyl, benzoxazolyl, benzthiazolyl, benztriazolyl, cinnolinyl, naphthyridinyl, phenanthridinyl, 7H-purinyl, 9H-purinyl, 6-amino-9H-purinyl, 5H-pyrrolo[3,2-d]pyrimidinyl, 7H-pyrrolo[2,3-d]pyrimidinyl, pyrido[2,3-d]pyrimidinyl, 2-phenylbenzo[d]thiazolyl, 1H-indolyl, 4,5,6,7-tetrahydro-1-H-indolyl, quinoxalinyl, 5-methylquinoxalinyl, quinazolinyl, quinolinyl, 8-hydroxy-quinolinyl, and isoquinolinyl.

One non-limiting example of a heteroaryl group as described above is C₁-C₅ heteroaryl, which has 1 to 5 carbon ring atoms and at least one additional ring atom that is a heteroatom (preferably 1 to 4 additional ring atoms that are heteroatoms) independently selected from nitrogen (N), oxygen (O), or sulfur (S). Examples of C₁-C₅ heteroaryl include, but are not limited to, triazinyl, thiazol-2-yl, thiazol-4-yl, imidazol-1-yl, 1H-imidazol-2-yl, 1H-imidazol-4-yl, isoxazolin-5-yl, furan-2-yl, furan-3-yl, thiophen-2-yl, thiophen-4-yl, pyrimidin-2-yl, pyrimidin-4-yl, pyrimidin-5-yl, pyridin-2-yl, pyridin-3-yl, and pyridin-4-yl.

Unless otherwise noted, when two substituents are taken together to form a ring having a specified number of ring atoms (e.g., R² and R³ taken together with the nitrogen (N) to which they are attached to form a ring having from 3 to 7 ring members), the ring can have carbon atoms and optionally one or more (e.g., 1 to 3) additional heteroatoms independently selected from nitrogen (N), oxygen (O), or sulfur (S). The ring can be saturated or partially saturated and can be optionally substituted.

For the purposed of the present disclosure fused ring units, as well as spirocyclic rings, bicyclic rings and the like, which comprise a single heteroatom will be considered to belong to the cyclic family corresponding to the heteroatom containing ring. For example, 1,2,3,4-tetrahydroquinoline having the formula:

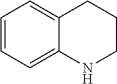

is, for the purposes of the present disclosure, considered a heterocyclic unit. 6,7-Dihydro-5H-cyclopentapyrimidine having the formula:

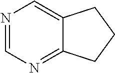

is, for the purposes of the disclosure, considered a heteroaryl unit. When a fused ring unit contains heteroatoms in both a saturated and an aryl ring, the aryl ring will predominate and determine the type of category to which the ring is assigned. For example, 1,2,3,4-tetrahydro-[1,8]naphthyridine having the formula:

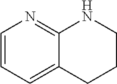

is, for the purposes of the present disclosure, considered a heteroaryl unit.

Whenever a term or either of their prefix roots appear in a name of a substituent the name is to be interpreted as including those limitations provided herein. For example, whenever the term “alkyl” or “aryl” or either of their prefix roots appear in a name of a substituent (e.g., arylalkyl, alkylamino) the name is to be interpreted as including those limitations given above for “alkyl” and “aryl.”

The term “substituted” is used throughout the specification. The term “substituted” is defined herein as a moiety, whether acyclic or cyclic, which has one or more hydrogen atoms replaced by a substituent or several (e.g., 1 to 10) substituents as defined herein below. The substituents are capable of replacing one or two hydrogen atoms of a single moiety at a time. In addition, these substituents can replace two hydrogen atoms on two adjacent carbons to form said substituent, new moiety or unit. For example, a substituted unit that requires a single hydrogen atom replacement includes halogen, hydroxyl, and the like. A two-hydrogen atom replacement includes carbonyl, oximino, and the like. A two-hydrogen atom replacement from adjacent carbon atoms includes epoxy, and the like. The term “substituted” is used throughout the present specification to indicate that a moiety can have one or more of the hydrogen atoms replaced by a substituent. When a moiety is described as “substituted” any number of the hydrogen atoms may be replaced. For example, difluoromethyl is a substituted C₁ alkyl; trifluoromethyl is a substituted C₁ alkyl; 4-hydroxyphenyl is a substituted aromatic ring; (N,N-dimethyl-5-amino)octanyl is a substituted C₈ alkyl; 3-guanidinopropyl is a substituted C₃ alkyl; and 2-carboxypyridinyl is a substituted heteroaryl.

The variable groups defined herein, e.g., alkyl, alkenyl, alkynyl, cycloalkyl, alkoxy, aryloxy, aryl, heterocycle and heteroaryl groups defined herein, whether used alone or as part of another group, can be optionally substituted. Optionally substituted groups will be so indicated.

The following are non-limiting examples of substituents which can substitute for hydrogen atoms on a moiety: halogen (chlorine (Cl), bromine (Br), fluorine (F) and iodine(I)), —CN, —NO₂, oxo (═O), —OR⁹, —SR⁹, —N(R⁹)₂, —NR⁹C(O)R⁹, —SO₂R⁹, —SO₂OR⁹, —SO₂N(R⁹)₂, —C(O)R⁹, —C(O)OR⁹, —C(O)N(R⁹)₂, C₁₋₆ alkyl, C₁₋₆ haloalkyl, C₁₋₆ alkoxy, C₂₋₈ alkenyl, C₂₋₈ alkynyl, C₃₋₁₄ cycloalkyl, aryl, heterocycle, or heteroaryl, wherein each of the alkyl, haloalkyl, alkenyl, alkynyl, alkoxy, cycloalkyl, aryl, heterocycle, and heteroaryl groups is optionally substituted with 1-10 (e.g., 1-6 or 1-4) groups selected independently from halogen, —CN, —NO₂, oxo, and R⁹; wherein R⁹, at each occurrence, independently is hydrogen, —OR¹⁰, —SR¹⁰, —C(O)R¹⁰, —C(O)OR¹⁰, —C(O)N(R¹⁰)₂, —SO₂R¹⁰, —S(O)₂OR¹⁰, —N(R¹⁰)₂, —NR¹⁰C(O)R¹⁰, C₁₋₆ alkyl, C₁₋₆ haloalkyl, C₂₋₈ alkenyl, C₂₋₈ alkynyl, cycloalkyl (e.g., C₃₋₆ cycloalkyl), aryl, heterocycle, or heteroaryl, or two R^{x} units taken together with the atom(s) to which they are bound form an optionally substituted carbocycle or heterocycle wherein said carbocycle or heterocycle has 3 to 7 ring atoms; wherein R¹⁰, at each occurrence, independently is hydrogen, C₁₋₆ alkyl, C₁₋₆ haloalkyl, C₂₋₈ alkenyl, C₂₋₈ alkynyl, cycloalkyl (e.g., C₃₋₆ cycloalkyl), aryl, heterocycle, or heteroaryl, or two R¹⁰ units taken together with the atom(s) to which they are bound form an optionally substituted carbocycle or heterocycle wherein said carbocycle or heterocycle preferably has 3 to 7 ring atoms.

In some embodiments, the substituents are selected from
i) —OR¹¹; for example, —OH, —OCH₃, —OCH₂CH₃, —OCH₂CH₂CH₃;
ii) —C(O)R¹¹; for example, —COCH₃, —COCH₂CH₃, —COCH₂CH₂CH₃;
iii) —C(O)OR¹¹; for example, —CO₂CH₃, —CO₂CH₂CH₃, —CO₂CH₂CH₂CH₃;
iv) —C(O)N(R¹¹)₂; for example, —CONH₂, —CONHCH₃, —CON(CH₃)₂;
v) —N(R¹¹)₂; for example, —NH₂, —NHCH₃, —N(CH₃)₂, —NH(CH₂CH₃);
vi) halogen: —F, —Cl, —Br, —I, or combinations thereof;
vii) —CHₑX_{g}; wherein X is halogen, m is from 0 to 2, e+g=3; for example, —CH₂F, —CHF₂, —CF₃, —CCl₃, or —CBr₃;
viii) —SO₂R¹¹; for example, —SO₂H; —SO₂CH₃; —SO₂C₆H₅;
ix) C₁-C₆ linear, branched, or cyclic alkyl;
x) Cyano
xi) Nitro;
xii) N(R¹¹)C(O)R¹¹;
xiii) Oxo (═O);
xiv) Heterocycle; and
xv) Heteroaryl.
   wherein each R¹¹ is independently hydrogen, optionally substituted C₁-C₆ linear or branched alkyl (e.g., optionally substituted C₁-C₄ linear or branched alkyl), or optionally substituted C₃-C₆cycloalkyl (e.g optionally substituted C₃-C₄ cycloalkyl); or two R¹¹ units can be taken together to forma ring comprising 3-7 ring atoms. In certain aspects, each R¹¹ is independently hydrogen, C₁-C₆ linear or branched alkyl optionally substituted with halogen or C₃-C₆ cycloalkyl or C₃-C₆ cycloalkyl.

At various places in the present specification, substituents of compounds are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆, alkyl.

For the purposes of the present disclosure, the terms “compound,” “analog,” and “composition of matter” stand equally well for the beta lactams described herein, including all enantiomeric forms, diastereomeric forms, salts, and the like, and the terms “compound,” “analog,” and “composition of matter” are used interchangeably throughout the present specification.

Compounds described herein can contain an asymmetric atom (also referred as a chiral center), and some of the compounds can contain one or more asymmetric atoms or centers, which can thus give rise to optical isomers (enantiomers) and diastereomers. The present disclosure and compounds disclosed herein include such enantiomers and diastereomers, as well as the racemic and resolved, enantiomerically pure R and S stereoisomers, as well as other mixtures of the R and S stereoisomers and pharmaceutically acceptable salts thereof. Optical isomers can be obtained in pure form by standard procedures known to those skilled in the art, which include, but are not limited to, diastereomeric salt formation, kinetic resolution, and asymmetric synthesis. The present disclosure also encompasses cis and trans isomers of compounds containing alkenyl moieties (e.g., alkenes and imines). It is also understood that the present disclosure encompasses all possible regioisomers, and mixtures thereof, which can be obtained in pure form by standard separation procedures known to those skilled in the art, and include, but are not limited to, column chromatography, thin-layer chromatography, and high-performance liquid chromatography.

Pharmaceutically acceptable salts of compounds of the present disclosure, which can have an acidic moiety, can be formed using organic and inorganic bases. Both mono and polyanionic salts are contemplated, depending on the number of acidic hydrogens available for deprotonation. Suitable salts formed with bases include metal salts, such as alkali metal or alkaline earth metal salts, for example sodium, potassium, or magnesium salts; ammonia salts and organic amine salts, such as those formed with morpholine, thiomorpholine, piperidine, pyrrolidine, a mono-, di- or tri-lower alkylamine (e.g., ethyl-tert-butyl-, diethyl-, diisopropyl-, triethyl-, tributyl- or dimethylpropylamine), or a mono-, di-, or trihydroxy lower alkylamine (e.g., mono-, di- or triethanolamine). Specific non-limiting examples of inorganic bases include NaHCO₃, Na₂CO₃, KHCO₃, K₂CO₃, Cs₂CO₃, LiOH, NaOH, KOH, NaH₂PO₄, Na₂HPO₄, and Na₃PO₄. Internal salts also can be formed. Similarly, when a compound disclosed herein contains a basic moiety, salts can be formed using organic and inorganic acids. For example, salts can be formed from the following acids: acetic, propionic, lactic, benzenesulfonic, benzoic, camphorsulfonic, citric, tartaric, succinic, dichloroacetic, ethenesulfonic, formic, fumaric, gluconic, glutamic, hippuric, hydrobromic, hydrochloric, isethionic, lactic, maleic, malic, malonic, mandelic, methanesulfonic, mucic, napthalenesulfonic, nitric, oxalic, pamoic, pantothenic, phosphoric, phthalic, propionic, succinic, sulfuric, tartaric, toluenesulfonic, and camphorsulfonic as well as other known pharmaceutically acceptable acids.

When any variable occurs more than one time in any constituent or in any formula, its definition in each occurrence is independent of its definition at every other occurrence (e.g., in N(R⁷)₂, each R⁷ may be the same or different than the other). Combinations of substituents and/or variables are permissible only if such combinations result in stable compounds.

As used herein, the term “therapeutic agent” refers to any molecule, compound, and/or substance that is used for the purpose of treating and/or managing a disease or disorder.

As used herein, the terms “therapies” and “therapy” can refer to any method(s), composition(s), and/or agent(s) that can be used in the prevention, treatment and/or management of a disease or condition, or one or more symptoms thereof. In certain embodiments, the terms “therapy” and “therapies” refer to small molecule therapy.

As used herein, the terms “treat,” “treatment,” and “treating” in the context of the administration of a therapy to a subject refer to the reduction or inhibition of the progression and/or duration of a disease or condition, the reduction or amelioration of the severity of a disease or condition, such as cancer, and/or the amelioration of one or more symptoms thereof resulting from the administration of one or more therapies. The terms “treat” and “treating” and “treatment” as used herein, refer to partially or completely alleviating, inhibiting, ameliorating and/or relieving a condition from which a patient is suspected to suffer.

As used herein, “therapeutically effective” and “effective dose” refer to a substance or an amount that elicits a desirable biological activity or effect.

Except when noted, the terms “subject” or “patient” are used interchangeably and refer to mammals such as human patients and non-human primates, as well as experimental animals such as rabbits, rats, and mice, and other animals. Accordingly, the term “subject” or “patient” as used herein means any mammalian patient or subject to which the compounds of the invention can be administered. In an exemplary embodiment of the present disclosure, to identify subject patients for treatment according to the methods of the invention, accepted screening methods are employed to determine risk factors associated with a targeted or suspected disease or condition or to determine the status of an existing disease or condition in a subject. These screening methods include, for example, conventional workups to determine risk factors that may be associated with the targeted or suspected disease or condition. These and other routine methods allow the clinician to select patients in need of therapy using the methods and compounds of the present disclosure.

As used herein, the term “excipient” refers to an inactive substance that serves as the vehicle or medium for a drug or other active substance. Examples of a suitable excipient include, but are not limited to, a solvent, a co-solvent, a coloring agent, a preservative, an antimicrobial agent, a filler, a binder, a disintegrate, a lubricant, a surfactant, an emulsifying agent, a suspending agent, or any combination thereof.

Studies have shown that chronic exposure to ethanol in alcohol preferring (P) rats resulted in reduced expression of glutamate transporter 1 (GLT-1) and cystine/glutamate exchanger transporter (xCT) and, as a result, increased extracellular glutamate concentration in the NAc. Moreover, treatment of GLT-1 modulators (e.g. Ceftriaxone and MS-153) upregulated GLT-1 and xCT expression in the NAc and attenuated ethanol drinking behaviors. These studies suggest that these glutamate transporters might be potential targets for the treatment of ethanol dependence. Other neuroadaptations thought to be involved in the development of ethanol dependence in animal models include the downregulation of metabotropic glutamate receptors subtype 5 (mGluR5) and its signaling pathway, which is proposed as a promising target for the treatment of SUDs. Together, these studies suggested that manipulation of glutamate transporters/receptors may serve as therapeutic target for the treatment of ethanol dependence.

Dysregulation of the glutamatergic systems in key brain reward regions such as the nucleus accumbens (NAc) is a consequence of chronic ethanol exposure, characterized by altered glutamate receptors and transporters. We showed that chronic exposure to ethanol in alcohol preferring (P) rats resulted in reduced expression of glutamate transporter 1 (GLT-1) and cystine/glutamate exchanger transporter (xCT) and, as a result, increased extracellular glutamate concentration in the NAc. In addition to brain effects, chronic ethanol consumption causes chronic liver injury associated with steatohepatitis. Additional studies have highlighted the role of liver-brain axis in ethanol dependence. Thus, this study we aimed in this study to determine the effect of chronic ethanol consumption on the glutamatergic system in the brain and liver. P rats were given voluntary access to ethanol and water for five weeks. Results showed that chronic ethanol drinking reduced the protein expression of GLT-1 and xCT in the NAc shell but not in the NAc core. Moreover, chronic ethanol consumption induced fatty liver with significantly higher levels of Peroxisome proliferator-activated receptor alpha (PPAR-α) and GLT-1 expression in the liver.

Structural modification of the carboxylic acid group and the 6-membered ring of ceftriaxone resulted in a novel beta-lactam compound such as MC-100093 as described herein, which shows enhanced GLT-1 upregulatory properties compared to ceftriaxone. We hypothesize in this study that treatment with novel beta-lactam compound, such as MC-100093, will attenuate ethanol drinking behavior as a result of GLT-1 upregulation. Using our rat model of chronic ethanol drinking (i.e., alcohol preferring ‘P rats’), we investigated the effects of chronic ethanol exposure and MC-100093 treatment on glutamatergic receptors and transporters as well as PPARs in the liver and brain.

Importantly, treatment with novel beta-lactam compound, MC-100093, attenuated ethanol drinking behavior and normalized levels of GLT-1, xCT and PPAR-α proteins in brain and liver. This study suggests that MC-100093 as a potential therapeutic drug for the treatment for alcohol dependence.

The present disclosure provides a composition comprising an effective amount of one or more compounds according to the present disclosure and at least one excipient, for treating, lessening effect of, or preventing alcohol dependence and/or alcohol related fatty liver diseases. In some embodiments, the one or more compounds are one or more beta lactam compounds or derivatives of formula (I),

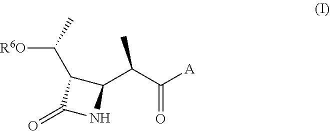

including hydrates, solvates, pharmaceutically acceptable salts, prodrugs, and complexes thereof, wherein:
A is selected from the group consisting of

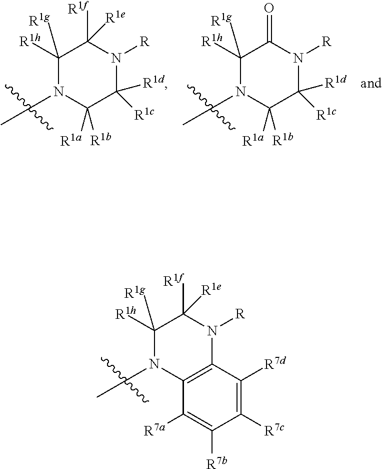

R is selected from the group consisting of hydrogen, C₁₋₆ linear alkyl, C₁₋₆ branched alkyl, optionally substituted aryl, C(O)R², C(O)OR³, C(O)NR^{4a}R^{4b}, SO₂R⁵, and SO₂NH₂;
R^{1a}, R^{1b}, R^{1c}, R^{1d}, R^{1e}, R^{1f}, R^{1g}, and R^{1h} are each independently selected from the group consisting of hydrogen, C₁₋₆ linear alkyl, and C₁₋₆ branch alkyl;
In some embodiments, R^{1b} and R^{1g} are joined together with the atoms to which they are bound to form a ring containing 5, 6, or 7 atoms;
In some embodiments, R^{1b} and R^{1f} are joined together with the atoms to which they are bound to form a ring containing 5, 6, or 7 atoms;
In some embodiments, R^{1d} and R^{1f} are joined together with the atoms to which they are bound to form a ring containing 5, 6, or 7 atoms;
In some embodiments, R^{1b} and R^{1c} are joined together with the atoms to which they are bound to form a ring containing 5 or 6 atoms;
R² is selected from the group consisting of C₁₋₆ linear alkyl, C₁₋₆ branched alkyl, and optionally substituted aryl;
R³ is selected from the group consisting of C₁₋₆ linear alkyl, C₁₋₆ branched alkyl, and optionally substituted aryl;
R^{4a} is selected from the group consisting of C₁₋₆ linear alkyl, C₁₋₆ branched alkyl, and optionally substituted aryl;
R^{4b} is selected from the group consisting of C₁₋₆ linear alkyl, C₁₋₆ branched alkyl, and optionally substituted aryl;
R⁵ is selected from the group consisting of C₁₋₆ linear alkyl, C₁₋₆ branched alkyl, and optionally substituted aryl;
R⁶ is selected from the group consisting of hydrogen, C₁₋₆ linear alkyl, and C(O)R⁸:
R^{7a}, R^{7b}, R^{7c}, and R^{7d} are each independently selected from the group consisting of are each independently selected from a group consisting of hydrogen, halogen, OH, C₁₋₆ linear alkyl, C₁₋₆ branched alkyl, C₁₋₆ alkoxy, C₁₋₆ haloalkyl, C₁₋₆ haloalkoxy, cyano, NH(C₁₋₆ alkyl), N(C₁₋₆ alkyl)₂, NHC(O)R⁸, C(O)NHR⁸, C(O)N(R⁸)₂, SH, SC₁₋₆ alkyl, SO₂NH₂, SO₂NHR⁸, SO₂R⁸, and NHSO₂R⁸;
R⁸ is independently selected at each occurrence from a group consisting of hydrogen, C₁₋₆ linear alkyl, C₁₋₆ branched alkyl, and C₃₋₇ cycloalkyl;

In some embodiments A is

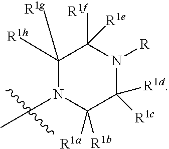

In some embodiments A is R

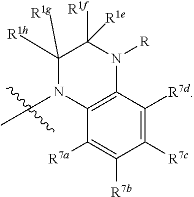

In some embodiments A is

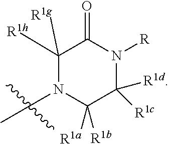

In some embodiments R is hydrogen.

In some embodiments R is C₁₋₆ linear alkyl.

In some embodiments R is C₁₋₆ branched alkyl.

In some embodiments R is optionally substituted aryl.

In some embodiments R is C(O)R².

In some embodiments R is C(O)OR³.

In some embodiments R is C(O)NR^{4a}R^{4b}.

In some embodiments R is SO₂R⁵.

In some embodiments R is SO₂NH₂.

In some embodiments R^{1a} is hydrogen.

In some embodiments R^{1a} is C₁₋₆ linear alkyl.

In some embodiments R^{1a} is C₁₋₆ branch alkyl.

In some embodiments R^{1b} is hydrogen.

In some embodiments R^{1b} is C₁₋₆ linear alkyl.

In some embodiments R^{1b} is C₁₋₆ branch alkyl.

In some embodiments R^{1c} is hydrogen.

In some embodiments R^{1c} is C₁₋₆ linear alkyl.

In some embodiments R^{1c} is C₁₋₆ branch alkyl.

In some embodiments R^{1d} is hydrogen.

In some embodiments R^{1d} is C₁₋₆ linear alkyl.

In some embodiments R^{1d} is C₁₋₆ branch alkyl.

In some embodiments R^{1e} is hydrogen.

In some embodiments R^{1e} is C₁₋₆ linear alkyl.

In some embodiments R^{1e} is C₁₋₆ branch alkyl.

In some embodiments R^{1f} is hydrogen.

In some embodiments R^{1f} is C₁₋₆ linear alkyl.

In some embodiments R^{1f} is C₁₋₆ branch alkyl.

In some embodiments R^{1g} is hydrogen.

In some embodiments R^{1g} is C₁₋₆ linear alkyl.

In some embodiments R^{1g} is C₁₋₆ branch alkyl.

In some embodiments R^{1h} is hydrogen.

In some embodiments R^{1h} is C₁₋₆ linear alkyl.

In some embodiments R^{1h} is C₁₋₆ branch alkyl.

In some embodiments R^{1b} and R^{1g} are joined together with the atoms to which they are bound to form a ring containing 5 atoms.

In some embodiments R^{1b} and R^{1g} are joined together with the atoms to which they are bound to form a ring containing 6 atoms.

In some embodiments R^{1b} and R^{1g} are joined together with the atoms to which they are bound to form a ring containing 7 atoms.

In some embodiments R^{1b} and R^{1f} are joined together with the atoms to which they are bound to form a ring containing 5 atoms.

In some embodiments R^{1b} and R^{1f} are joined together with the atoms to which they are bound to form a ring containing 6 atoms.

In some embodiments R^{1b} and R^{1f} fare joined together with the atoms to which they are bound to form a ring containing 7 atoms.

In some embodiments R^{1d} and R^{1f} are joined together with the atoms to which they are bound to form a ring containing 5 atoms

In some embodiments R^{1d} and R^{1f} are joined together with the atoms to which they are bound to form a ring containing 6 atoms.

In some embodiments R^{1d} and R^{1f} are joined together with the atoms to which they are bound to form a ring containing 7 atoms.

In some embodiments R^{1b} and R^{1c} are joined together with the atoms to which they are bound to form a ring containing 5 atoms.

In some embodiments R^{1b} and R^{1c} are joined together with the atoms to which they are bound to form a ring containing 6 atoms.

In some embodiments R² is C₁₋₆ linear alkyl.

In some embodiments R² is C₁₋₆ branched alkyl.

In some embodiments R² is optionally substituted aryl.

In some embodiments R³ is C₁₋₆ linear alkyl.

In some embodiments R³ is C₁₋₆ branched alkyl.

In some embodiments R³ is optionally substituted aryl.

In some embodiments R^{4a} is C₁₋₆ linear alkyl.

In some embodiments R^{4a} is C₁₋₆ branched alkyl.

In some embodiments R^{4a} is optionally substituted aryl.

In some embodiments R^{4b} is C₁₋₆ linear alkyl.

In some embodiments R^{4b} is C₁₋₆ branched alkyl.

In some embodiments R^{4b} is optionally substituted aryl.

In some embodiments R⁵ is C₁₋₆ linear alkyl.

In some embodiments R⁵ is C₁₋₆ branched alkyl.

In some embodiments R⁵ is optionally substituted aryl.

In some embodiments R⁶ is hydrogen.

In some embodiments R⁶ is C₁₋₆ linear alkyl.

In some embodiments R⁶ is C(O)R⁸.

In some embodiments R^{7a} is hydrogen.

In some embodiments R^{7a} is halogen.

In some embodiments R^{7a} is OH.

In some embodiments R^{7a} is C₁₋₆ linear alkyl.

In some embodiments R^{7a} is C₁₋₆ branched alkyl.

In some embodiments R^{7a} is C₁₋₆ alkoxy.

In some embodiments R^{7a} is C₁₋₆ haloalkyl.

In some embodiments R^{7a} is C₁₋₆ haloalkoxy.

In some embodiments R^{7a} is cyano.

In some embodiments R^{7a} is NH(C₁₋₆ alkyl).

In some embodiments R^{7a} is N(C₁₋₆ alkyl)₂.

In some embodiments R^{7a} is NHC(O)R⁸.

In some embodiments R^{7a} is C(O)NHR⁸.

In some embodiments R^{7a} is C(O)N(R⁸)₂.

In some embodiments R^{7a} is SH.

In some embodiments R^{7a} is SC₁₋₆ alkyl.

In some embodiments R^{7a} is SO₂NH₂.

In some embodiments R^{7a} is SO₂NHR¹.

In some embodiments R^{7a} is SO₂R⁸.

In some embodiments R^{7a} is NHSO₂R⁸.

In some embodiments R^{7b} is hydrogen.

In some embodiments R^{7b} is halogen.

In some embodiments R^{7b} is OH.

In some embodiments R^{7b} is C₁₋₆ linear alkyl.

In some embodiments R^{7b} is C₁₋₆ branched alkyl.

In some embodiments R^{7b} is C₁₋₆ alkoxy.

In some embodiments R^{7b} is C₁₋₆ haloalkyl.

In some embodiments R^{7b} is C₁₋₆ haloalkoxy.

In some embodiments R^{7b} is cyano.

In some embodiments R^{7b} is NH(C₁₋₆ alkyl).

In some embodiments R^{7b} is N(C₁₋₆ alkyl)₂.

In some embodiments R^{7b} is NHC(O)R⁸.

In some embodiments R^{7b} is C(O)NHR⁸.

In some embodiments R^{7b} is C(O)N(R⁸)₂.

In some embodiments R^{7b} is SH.

In some embodiments R^{7b} is SC₁₋₆ alkyl.

In some embodiments R^{7b} is SO₂NH₂.

In some embodiments R^{7b} is SO₂NHR⁸.

In some embodiments R^{7b} is SO₂R⁸.

In some embodiments R^{7b} is NHSO₂R⁸.

In some embodiments R^{7C} is hydrogen.

In some embodiments R^{7C} is halogen.

In some embodiments R^{7C} is OH.

In some embodiments R^{7C} is C₁₋₆ linear alkyl.

In some embodiments R^{7C} is C₁₋₆ branched alkyl.

In some embodiments R^{7C} is C₁₋₆ alkoxy.

In some embodiments R^{7C} is C₁₋₆ haloalkyl.

In some embodiments R^{7C} is C₁₋₆ haloalkoxy.

In some embodiments R^{7C} is cyano.

In some embodiments R^{7C} is NH(C₁₋₆ alkyl).

In some embodiments R^{7C} is N(C₁₋₆ alkyl)₂.

In some embodiments R^{7c} is NHC(O)R⁸.

In some embodiments R^{7c} is C(O)NHR⁸.

In some embodiments R^{7C} is C(O)N(R⁸)₂.

In some embodiments R^{7C} is SH.

In some embodiments R^{7c} is SC₁₋₆ alkyl.

In some embodiments R^{7c} is SO₂NH₂.

In some embodiments R^{7c} is SO₂NHR¹.

In some embodiments R^{7c} is SO₂R⁸.

In some embodiments R^{7c} is NHSO₂R⁸.

In some embodiments R^{7d} is hydrogen.

In some embodiments R^{7d} is halogen.

In some embodiments R^{7d} is OH.

In some embodiments R^{7d} is C₁₋₆ linear alkyl.

In some embodiments R^{7d} is C₁₋₆ branched alkyl.

In some embodiments R^{7d} is C₁₋₆ alkoxy.

In some embodiments R^{7d} is C₁₋₆ haloalkyl.

In some embodiments R^{7d} is C₁₋₆ haloalkoxy.

In some embodiments R^{7d} is cyano.

In some embodiments R^{7d} is NH(C₁₋₆ alkyl).

In some embodiments R^{7d} is N(C₁₋₆ alkyl)₂.

In some embodiments R^{7d} is NHC(O)R⁸.

In some embodiments R^{7d} is C(O)NHR¹.

In some embodiments R^{7d} is C(O)N(R⁸)₂.

In some embodiments R^{7d} is SH.

In some embodiments R^{7d} is SC₁₋₆ alkyl.

In some embodiments R^{7d} is SO₂NH₂.

In some embodiments R^{7d} is SO₂NHR⁸.

In some embodiments R^{7d} is SO₂R⁸.

In some embodiments R^{7d} is NHSO₂R⁸.

In some embodiments R⁸ is hydrogen.

In some embodiments R⁸ is C₁₋₆ linear alkyl.

In some embodiments R⁸ is C₁₋₆ branched alkyl.

In some embodiments R⁸ is C₃₋₇ cycloalkyl.

Exemplary embodiments include compounds having the formula (I) or a pharmaceutically acceptable salt form thereof:

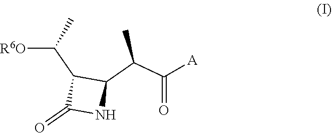

wherein non-limiting examples of R⁶ and A are defined herein below in Table 1.

| Entry | R⁶ | A |
|---|---|---|
| 1 | H | 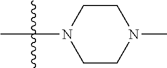 [SMILES: *N1CCN(C)CC1] |
| 2 | H | 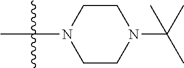 [SMILES: *N1CCN(C(C)(C)C)CC1] |
| 3 | H | 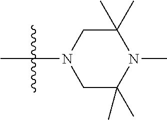 [SMILES: *N1CC(C)(C)N(C)C(C)(C)C1] |
| 4 | H | 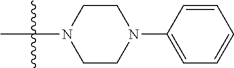 [SMILES: *N1CCN(c2ccccc2)CC1] |
| 5 | H | 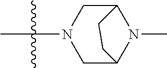 [SMILES: *N1CC2CCC(C1)N2C] |
| 6 | H | 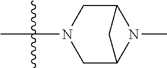 [SMILES: *N1CC2CC(C1)N2C] |
| 7 | CH₃ | 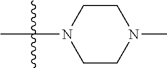 [SMILES: *N1CCN(C)CC1] |
| 8 | CH₃ | 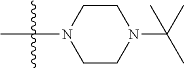 [SMILES: *N1CCN(C(C)(C)C)CC1] |
| 9 | CH₃ | 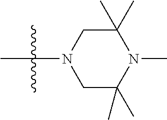 [SMILES: *N1CC(C)(C)N(C)C(C)(C)C1] |
| 10 | CH₃ | 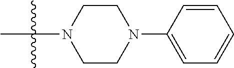 [SMILES: *N1CCN(c2ccccc2)CC1] |
| 11 | CH₃ | 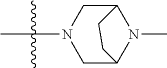 [SMILES: *N1CC2CCC(C1)N2C] |
| 12 | CH₃ | 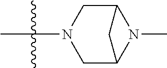 [SMILES: *N1CC2CC(C1)N2C] |
| 13 | CH₃C(═O) | 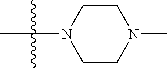 [SMILES: *N1CCN(C)CC1] |
| 14 | CH₃C(═O) | 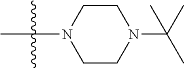 [SMILES: *N1CCN(C(C)(C)C)CC1] |
| 15 | CH₃C(═O) | 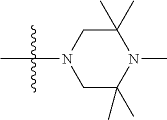 [SMILES: *N1CC(C)(C)N(C)C(C)(C)C1] |
| 16 | CH₃C(═O) | 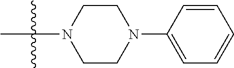 [SMILES: *N1CCN(c2ccccc2)CC1] |
| 17 | CH₃C(═O) | 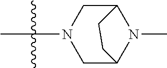 [SMILES: *N1CC2CCC(C1)N2C] |
| 18 | CH₃C(═O) | 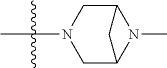 [SMILES: *N1CC2CC(C1)N2C] |
| 19 | H | 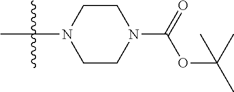 [SMILES: *N1CCN(C(=O)OC(C)(C)C)CC1] |
| 20 | H | 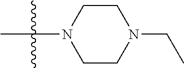 [SMILES: *N1CCN(CC)CC1] |
| 21 | H | 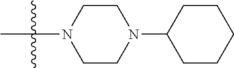 [SMILES: *N1CCN(C2CCCCC2)CC1] |
| 22 | H | 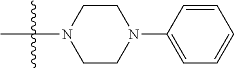 [SMILES: *N1CCN(c2ccccc2)CC1] |
| 23 | (CH₃)₂CHC(═O) | 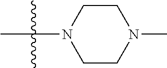 [SMILES: *N1CCN(C)CC1] |
| 24 | CH₃CH₂C(═O) | 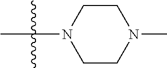 [SMILES: *N1CCN(C)CC1] |
| 25 | CH₃(CH₂)₂C(═O) | 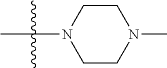 [SMILES: *N1CCN(C)CC1] |
| 26 | H | 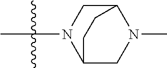 [SMILES: *N1CC2CCC1CN2C] |
| 27 | H | 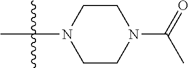 [SMILES: *N1CCN(C(C)=O)CC1] |
| 28 | H | 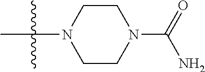 [SMILES: *N1CCN(C(N)=O)CC1] |
| 29 | H | 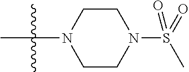 [SMILES: *N1CCN(S(C)(=O)=O)CC1] |
| 30 | H | 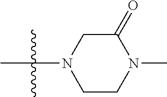 [SMILES: *N1CCN(C)C(=O)C1] |
| 31 | H | 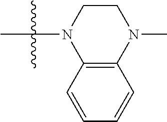 [SMILES: *N1CCN(C)c2ccccc21] |
| 32 | CH₃ | 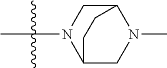 [SMILES: *N1CC2CCC1CN2C] |
| 33 | CH₃ | 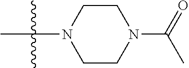 [SMILES: *N1CCN(C(C)=O)CC1] |
| 34 | CH₃ | 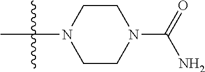 [SMILES: *N1CCN(C(N)=O)CC1] |
| 35 | CH₃ | 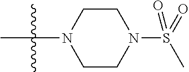 [SMILES: *N1CCN(S(C)(=O)=O)CC1] |
| 36 | CH₃ | 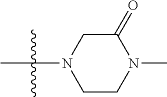 [SMILES: *N1CCN(C)C(=O)C1] |
| 37 | CH₃ | 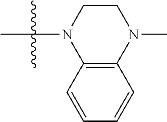 [SMILES: *N1CCN(C)c2ccccc21] |
| 38 | CH₃C(═O) | 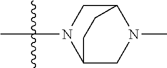 [SMILES: *N1CC2CCC1CN2C] |
| 39 | CH₃C(═O) | 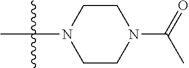 [SMILES: *N1CCN(C(C)=O)CC1] |
| 40 | CH₃C(═O) | 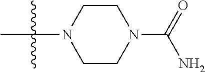 [SMILES: *N1CCN(C(N)=O)CC1] |
| 41 | CH₃C(═O) | 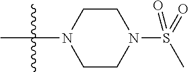 [SMILES: *N1CCN(S(C)(=O)=O)CC1] |
| 42 | CH₃C(═O) | 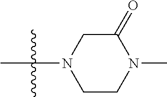 [SMILES: *N1CCN(C)C(=O)C1] |
| 43 | CH₃C(═O) | 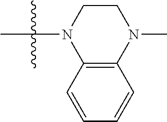 [SMILES: *N1CCN(C)c2ccccc21] |
| 44 | H | 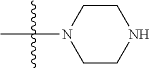 [SMILES: *N1CCNCC1] |
| 45 | (CH₃)₃C(═O) | 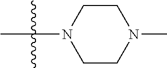 [SMILES: *N1CCN(C)CC1] |
| 46 | H | 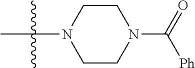 [SMILES: *N1CCN(C(=O)c2ccccc2)CC1] |
| 47 | H | 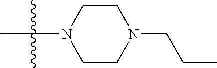 [SMILES: *N1CCN(CCC)CC1] |
| 48 | H | 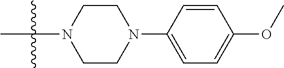 [SMILES: *N1CCN(c2ccc(OC)cc2)CC1] |

For the purposes of demonstrating the manner in which the compounds of the present disclosure are named and referred to herein, the compound having the formula:

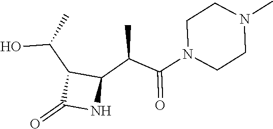 [SMILES: C[C@@H](O)[C@H]1C(=O)N[C@@H]1[C@@H](C)C(=O)N1CCN(C)CC1]

has the chemical name (3S,4R)-3-((R)-1-hydroxyethyl)-4-((R)-1-(4-methylpiperazin-1-yl)-1-oxopropan-2-yl)azetidin-2-one.

For the purposes of the present disclosure, a compound depicted by the racemic formula, for example:

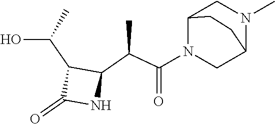 [SMILES: C[C@@H](O)[C@H]1C(=O)N[C@@H]1[C@@H](C)C(=O)N1CC2CCC1CN2C]

will stand equally well for either of the two enantiomers having the formula:

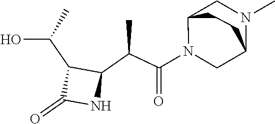 [SMILES: C[C@@H](O)[C@H]1C(=O)N[C@@H]1[C@@H](C)C(=O)N1C[C@@H]2CC[C@H]1CN2C]

or the formula:

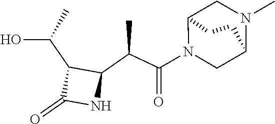

or mixtures thereof, or in the case where a second chiral center is present, all diastereomers.

In all of the embodiments provided herein, examples of suitable optional substituents are not intended to limit the scope of the claimed invention. The compounds of the invention may contain any of the substituents, or combinations of substituents, provided herein.

### Process

The present disclosure further provides a process for preparing a composition comprising at least one beta lactams of the present disclosure.

Compounds of the present disclosure can be prepared in accordance with the procedures outlined herein, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field. It will be appreciated that where typical or preferred process conditions (i.e., reaction temperatures, times, mole ratios of reactants, solvents, pressures, etc.) are given, other process conditions can also be used unless otherwise stated. Optimum reaction conditions can vary with the particular reactants or solvent used, but such conditions can be determined by one skilled in the art by routine optimization procedures. Those skilled in the art of organic synthesis will recognize that the nature and order of the synthetic steps presented can be varied for the purpose of optimizing the formation of the compounds described herein.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatography such as high-pressure liquid chromatograpy (HPLC), gas chromatography (GC), gel-permeation chromatography (GPC), or thin layer chromatography (TLC).

Preparation of the compounds can involve protection and deprotection of various chemical groups. The need for protection and deprotection and the selection of appropriate protecting groups can be readily determined by one skilled in the art. The chemistry of protecting groups can be found, for example, in Greene et al., Protective Groups in Organic Synthesis, 2d. Ed. (Wiley & Sons, 1991), the entire disclosure of which is incorporated by reference herein for all purposes.

The reactions or the processes described herein can be carried out in suitable solvents which can be readily selected by one skilled in the art of organic synthesis. Suitable solvents typically are substantially nonreactive with the reactants, intermediates, and/or products at the temperatures at which the reactions are carried out, i.e., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected.

The compounds of these teachings can be prepared by methods known in the art of organic chemistry. The reagents used in the preparation of the compounds of these teachings can be either commercially obtained or can be prepared by standard procedures described in the literature. For example, compounds of the present disclosure can be prepared according to the method illustrated in the General Synthetic Schemes:

### General Synthetic Schemes for Preparation of Compounds.

The reagents used in the preparation of the compounds of this invention can be either commercially obtained or can be prepared by standard procedures described in the literature. In accordance with this invention, compounds in the genus may be produced by one of the following reaction schemes.

Compounds of formula (4) may be prepared according to the process outlined in Scheme 1.

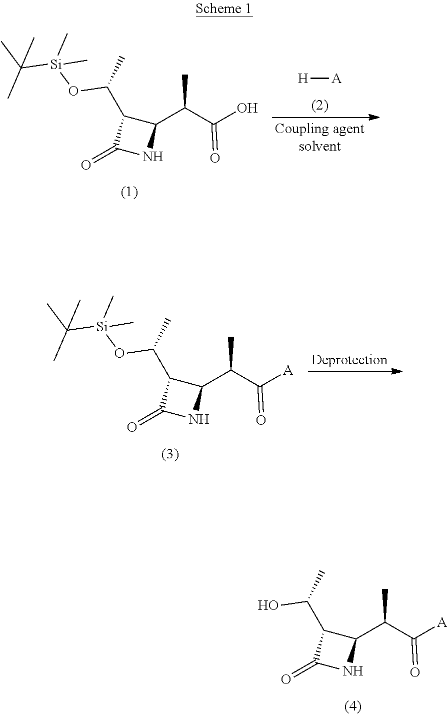

A compound of the formula (1) is reacted with a compound of the formula (2), a known compound or a compound prepared using known methods, in the presence of a coupling agent such as 1-ethyl-3-(3-dimethylaminopropyl) carbodiimide, N,N′-Dicyclohexylcarbodiimide, O-Benzotriazole-N,N,N′,N′-tetramethyl-uronium-hexafluoro-phosphate, 0-(7-azabenzotriazol-1-yl)-N,N,N′,N′-tetramethyluronium hexafluorophosphate, Benzotriazole-1-yl-oxy-tris-(dimethylamino)-phosphonium hexafluorophosphate, benzotriazol-1-yl-oxytripyrrolidinophosphonium hexafluorophosphate, and the like, in an organic solvent such as tetrahydronfuran, 1,4-dioxane, dimethylformamide, methylene chloride, 1,2-dichloroethane, methanol, ethanol, acetonitrile, and the like, optionally in the presence of a base such as triethylamine, diisopropylethylamine, pyridine, 2,6-lutidine, and the like, optionally in the presence of 4-N,N-dimethylaminopyridine, to provide a compound of the formula (3). A compound of the formula (3) is then with hydrogen fluoride in pyridine, optionally in a solvent such as tetrahydronfuran, 1,4-dioxane, dimethylformamide, methylene chloride, 1,2-dichloroethane, methanol, ethanol, acetonitrile, and the like to provide a compound of the formula (4). Alternatively, a compound of the formula (3) is reacted with hydrogen fluoride in triethylamine, optionally in a solvent such as tetrahydronfuran, 1,4-dioxane, dimethylformamide, methylene chloride, 1,2-dichloroethane, methanol, ethanol, acetonitrile, and the like to provide a compound of the formula (4). Alternatively, a compound of the formula (3) is reacted with an acid such as hydrochloric acid, sulfuric acid, trifluoroacetic acid, acetic acid, and the like, optionally in a solvent such as tetrahydronfuran, 1,4-dioxane, dimethylformamide, methylene chloride, 1,2-dichloroethane, methanol, ethanol, acetonitrile, and the like to provide a compound of the formula (4).

Compounds of formula (10) may be prepared according to the process outlined in Scheme 2.

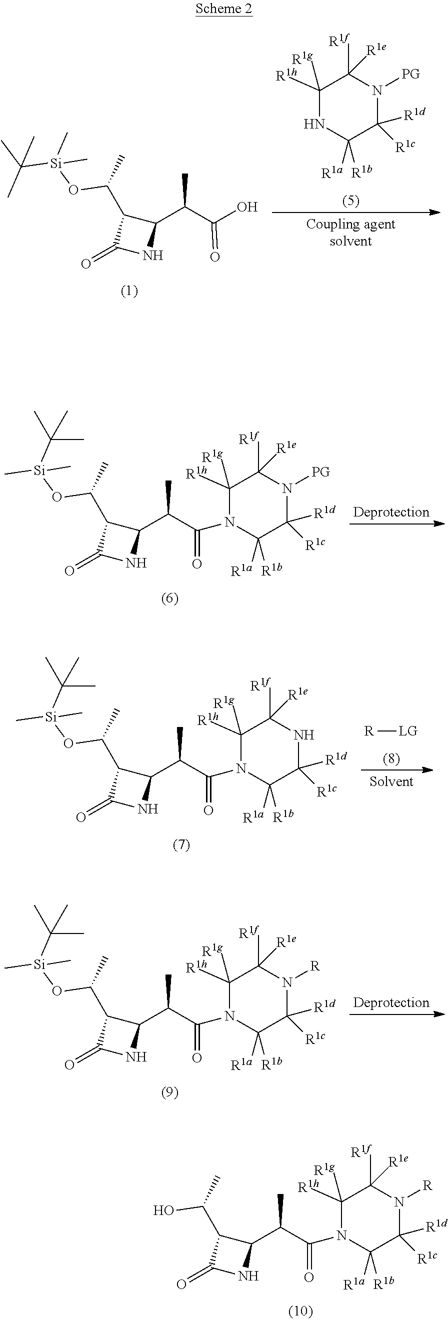

A compound of the formula (1) is reacted with a compound of the formula (5), a known compound or a compound prepared using known methods, wherein PG is a protecting group such as t-butyl carbamate, benzyl carbamate, 9-fluorenylmethyl carbamate, and the like, in the presence of a coupling agent such as 1-ethyl-3-(3-dimethylaminopropyl) carbodiimide, N,N′-Dicyclohexylcarbodiimide, O-Benzotriazole-N,N,N′,N′-tetramethyl-uronium-hexafluoro-phosphate, 0-(7-azabenzotriazol-1-yl)-N,N,N′,N′-tetramethyluronium hexafluorophosphate, Benzotriazole-1-yl-oxy-tris-(dimethylamino)-phosphonium hexafluorophosphate, benzotriazol-1-yl-oxytripyrrolidinophosphonium hexafluorophosphate, and the like, in an organic solvent such as tetrahydronfuran, 1,4-dioxane, dimethylformamide, methylene chloride, 1,2-dichloroethane, methanol, ethanol, acetonitrile, and the like, optionally in the presence of a base such as triethylamine, diisopropylethylamine, pyridine, 2,6-lutidine, and the like, optionally in the presence of 4-N,N-dimethylaminopyridine, to provide a compound of the formula (6). A compound of the formula (6) is then reacted with an acid such as hydrochloric acid, sulfuric acid, trifluoroacetic acid, acetic acid, and the like, optionally in a solvent such as tetrahydronfuran, 1,4-dioxane, dimethylformamide, methylene chloride, 1,2-dichloroethane, methanol, ethanol, acetonitrile, and the like to provide a compound of the formula (7). Alternatively, a compound of the formula (6) is reacted with hydrogen in the presence of a palladium catalyst such as palladium on carbon, palladium acetate, palladium bis(triphenylphosphine) dichloride, palladium tetrakis(triphenylphospine), bis(acetonitrile), and the like, in an organic solvent such as methanol, ethanol, isopropanol, ethyl acetate, tetrahydrofuran, 1,4-dioxane, and the like to provide a compound of the formula (7). Alternatively, a compound of the formula (6) is reacted with a base such as piperidine, pyridine, triethylamine, and the like, optionally in the presence of a solvent such as tetrahydronfuran, 1,4-dioxane, dimethylformamide, methylene chloride, 1,2-dichloroethane, methanol, ethanol, acetonitrile, and the like to provide a compound of the formula (7). A compound of the formula (7) is then reacted with a compound of the formula (8), a known compound or a compound prepared by known means, wherein LG is a leaving group such as chloride, bromide, methanesulfonate, tosylate, and the like, in the presence of a base such as triethylamine, diisopropylethylamine, pyridine, and the like, in the presence of a solvent such as tetrahydronfuran, 1,4-dioxane, dimethylformamide, methylene chloride, 1,2-dichloroethane, methanol, ethanol, acetonitrile, and the like to provide a compound of the formula (9). A compound of the formula (9) is then with hydrogen fluoride in pyridine, optionally in a solvent such as tetrahydronfuran, 1,4-dioxane, dimethylformamide, methylene chloride, 1,2-dichloroethane, methanol, ethanol, acetonitrile, and the like to provide a compound of the formula (10). Alternatively, a compound of the formula (9) is reacted with hydrogen fluoride in triethylamine, optionally in a solvent such as tetrahydronfuran, 1,4-dioxane, dimethylformamide, methylene chloride, 1,2-dichloroethane, methanol, ethanol, acetonitrile, and the like to provide a compound of the formula (10). Alternatively, a compound of the formula (9) is reacted with an acid such as hydrochloric acid, sulfuric acid, trifluoroacetic acid, acetic acid, and the like, optionally in a solvent such as tetrahydronfuran, 1,4-dioxane, dimethylformamide, methylene chloride, 1,2-dichloroethane, methanol, ethanol, acetonitrile, and the like to provide a compound of the formula (10).

Compounds of formula (13) may be prepared according to the process outlined in Scheme 3.

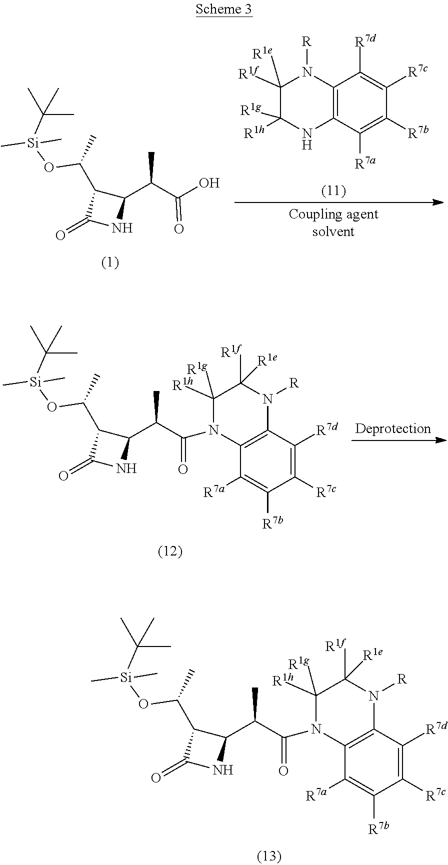

A compound of the formula (1) is reacted with a compound of the formula (11), a known compound or a compound prepared using known methods, in the presence of a coupling agent such as 1-ethyl-3-(3-dimethylaminopropyl) carbodiimide, N,N′-dicyclohexylcarbodiimide, O-benzotriazole-N,N,N′,N′-tetramethyl-uronium-hexafluoro-phosphate, 0-(7-azabenzotriazol-1-yl)-N,N,N′,N′-tetramethyluronium hexafluorophosphate, benzotriazole-1-yl-oxy-tris-(dimethylamino)-phosphonium hexafluorophosphate, benzotriazol-1-yl-oxytripyrrolidinophosphonium hexafluorophosphate, and the like, in an organic solvent such as tetrahydronfuran, 1,4-dioxane, dimethylformamide, methylene chloride, 1,2-dichloroethane, methanol, ethanol, acetonitrile, and the like, optionally in the presence of a base such as triethylamine, diisopropylethylamine, pyridine, 2,6-lutidine, and the like, optionally in the presence of 4-N,N-dimethylaminopyridine, to provide a compound of the formula (12). A compound of the formula (12) is then with hydrogen fluoride in pyridine, optionally in a solvent such as tetrahydronfuran, 1,4-dioxane, dimethylformamide, methylene chloride, 1,2-dichloroethane, methanol, ethanol, acetonitrile, and the like to provide a compound of the formula (13). Alternatively, a compound of the formula (12) is reacted with hydrogen fluoride in triethylamine, optionally in a solvent such as tetrahydronfuran, 1,4-dioxane, dimethylformamide, methylene chloride, 1,2-dichloroethane, methanol, ethanol, acetonitrile, and the like to provide a compound of the formula (13). Alternatively, a compound of the formula (12) is reacted with an acid such as hydrochloric acid, sulfuric acid, trifluoroacetic acid, acetic acid, and the like, optionally in a solvent such as tetrahydronfuran, 1,4-dioxane, dimethylformamide, methylene chloride, 1,2-dichloroethane, methanol, ethanol, acetonitrile, and the like to provide a compound of the formula (13).

Compounds of formula (19) may be prepared according to the process outlined in Scheme 4.

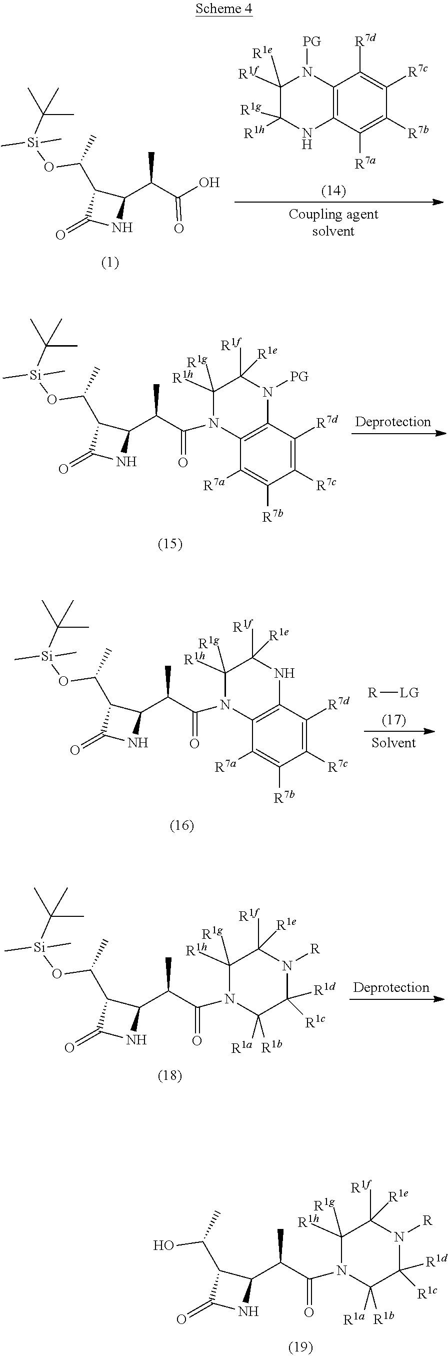

A compound of the formula (1) is reacted with a compound of the formula (14), a known compound or a compound prepared using known methods, wherein PG is a protecting group such as t-butyl carbamate, benzyl carbamate, 9-fluorenylmethyl carbamate, and the like, in the presence of a coupling agent such as 1-ethyl-3-(3-dimethylaminopropyl) carbodiimide, N,N′-dicyclohexylcarbodiimide, O-benzotriazole-N,N,N′,N′-tetramethyl-uronium-hexafluoro-phosphate, 0-(7-azabenzotriazol-1-yl)-N,N,N′,N′-tetramethyluronium hexafluorophosphate, benzotriazole-1-yl-oxy-tris-(dimethylamino)-phosphonium hexafluorophosphate, benzotriazol-1-yl-oxytripyrrolidinophosphonium hexafluorophosphate, and the like, in an organic solvent such as tetrahydronfuran, 1,4-dioxane, dimethylformamide, methylene chloride, 1,2-dichloroethane, methanol, ethanol, acetonitrile, and the like, optionally in the presence of a base such as triethylamine, diisopropylethylamine, pyridine, 2,6-lutidine, and the like, optionally in the presence of 4-N,N-dimethylaminopyridine, to provide a compound of the formula (15). A compound of the formula (15) is then reacted with an acid such as hydrochloric acid, sulfuric acid, trifluoroacetic acid, acetic acid, and the like, optionally in a solvent such as tetrahydronfuran, 1,4-dioxane, dimethylformamide, methylene chloride, 1,2-dichloroethane, methanol, ethanol, acetonitrile, and the like to provide a compound of the formula (16). Alternatively, a compound of the formula (15) is reacted with hydrogen in the presence of a palladium catalyst such as palladium on carbon, palladium acetate, palladium bis(triphenylphosphine) dichloride, palladium tetrakis(triphenylphospine), bis(acetonitrile), and the like, in an organic solvent such as methanol, ethanol, isopropanol, ethyl acetate, tetrahydrofuran, 1,4-dioxane, and the like to provide a compound of the formula (16). Alternatively, a compound of the formula (15) is reacted with a base such as piperidine, pyridine, triethylamine, and the like, optionally in the presence of a solvent such as tetrahydronfuran, 1,4-dioxane, dimethylformamide, methylene chloride, 1,2-dichloroethane, methanol, ethanol, acetonitrile, and the like to provide a compound of the formula (16). A compound of the formula (16) is then reacted with a compound of the formula (17), a known compound or a compound prepared by known means, wherein LG is a leaving group such as chloride, bromide, methanesulfonate, tosylate, and the like, in the presence of a base such as triethylamine, diisopropylethylamine, pyridine, and the like, in the presence of a solvent such as tetrahydronfuran, 1,4-dioxane, dimethylformamide, methylene chloride, 1,2-dichloroethane, methanol, ethanol, acetonitrile, and the like to provide a compound of the formula (18). A compound of the formula (18) is then with hydrogen fluoride in pyridine, optionally in a solvent such as tetrahydronfuran, 1,4-dioxane, dimethylformamide, methylene chloride, 1,2-dichloroethane, methanol, ethanol, acetonitrile, and the like to provide a compound of the formula (19). Alternatively, a compound of the formula (18) is reacted with hydrogen fluoride in triethylamine, optionally in a solvent such as tetrahydronfuran, 1,4-dioxane, dimethylformamide, methylene chloride, 1,2-dichloroethane, methanol, ethanol, acetonitrile, and the like to provide a compound of the formula (19). Alternatively, a compound of the formula (18) is reacted with an acid such as hydrochloric acid, sulfuric acid, trifluoroacetic acid, acetic acid, and the like, optionally in a solvent such as tetrahydronfuran, 1,4-dioxane, dimethylformamide, methylene chloride, 1,2-dichloroethane, methanol, ethanol, acetonitrile, and the like to provide a compound of the formula (19).

A compound of the formula (20) may be prepared according to the process outlined in Scheme 5.

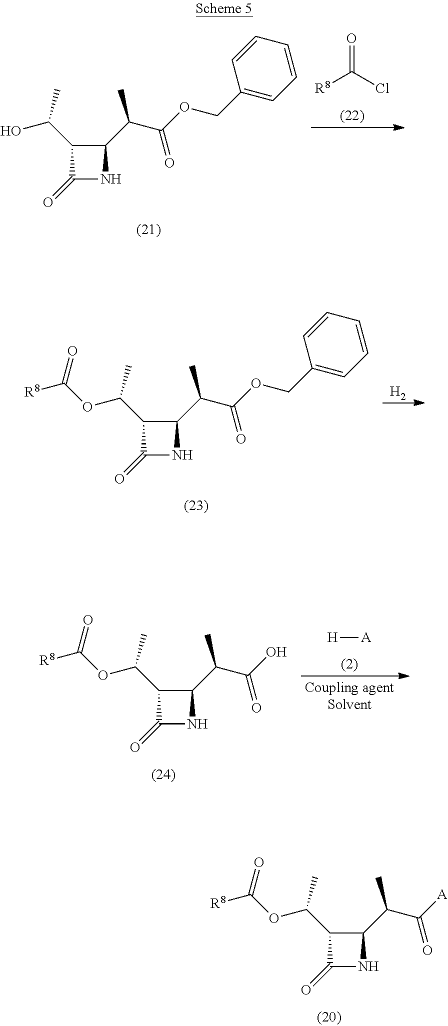

A compound of the formula (21) is reacted with a compound of the formula (22), a known compound or a compound prepared using known methods, optionally in the presence of a base such as triethylamine, diisopropylethylamine, pyridine, 2,6-lutidine, and the like, optionally in the presence of 4-N,N-dimethylaminopyridine, in an organic solvent such as tetrahydronfuran, 1,4-dioxane, dimethylformamide, methylene chloride, 1,2-dichloroethane, acetonitrile, and the like to provide a compound of the formula (23). A compound of the formula (23) is reacted with hydrogen in the presence of a palladium catalyst such as palladium on carbon, palladium acetate, palladium bis(triphenylphosphine) dichloride, palladium tetrakis(triphenylphospine), bis(acetonitrile), and the like, in an organic solvent such as methanol, ethanol, isopropanol, ethyl acetate, tetrahydrofuran, 1,4-dioxane, and the like to provide a compound of the formula (24). A compound of the formula (24) is reacted with a compound of the formula (2), a known compound or a compound prepared using known methods, in the presence of a coupling agent such as 1-ethyl-3-(3-dimethylaminopropyl) carbodiimide, N,N′-Dicyclohexylcarbodiimide, O-Benzotriazole-N,N,N′,N′-tetramethyl-uronium-hexafluoro-phosphate, 0-(7-azabenzotriazol-1-yl)-N,N,N′,N′-tetramethyl uronium hexafluorophosphate, Benzotriazole-1-yl-oxy-tris-(dimethylamino)-phosphonium hexafluorophosphate, benzotriazol-1-yl-oxytripyrrolidinophosphonium hexafluorophosphate, 4-(4,6-dimethoxy-1,3,5-triazin-2-yl)-4-methyl morpholinium chloride and the like, in an organic solvent such as tetrahydronfuran, 1,4-dioxane, dimethylformamide, methylene chloride, 1,2-dichloroethane, methanol, ethanol, acetonitrile, and the like, optionally in the presence of a base such as triethylamine, diisopropylethylamine, pyridine, 2,6-lutidine, and the like, optionally in the presence of 4-N,N-dimethylaminopyridine, to provide a compound of the formula (20).

The Examples provided below provide representative methods for preparing exemplary compounds of the present disclosure. The skilled practitioner will know how to substitute the appropriate reagents, starting materials and purification methods known to those skilled in the art, in order to prepare the compounds of the present disclosure. [0254]¹H-NMR spectra were obtained on a Bruker 400-MHz NMR. Purity (%) and mass spectral data were determined with an Agilent Technologies HPLC/MS (Zorbax SB-C18, 2.1×30 mm, 3.5 μm) with a diode array detector from 210-400 nm.

### EXAMPLES OF COMPOUNDS

Example 1 provides methods for preparing representative compounds of formula (I). The skilled practitioner will know how to substitute the appropriate reagents, starting materials and purification methods known to those skilled in the art, in order to prepare additional compounds of the present disclosure.

### Example 1: Synthesis of (3S, 4R)-3-((R)-(1-hydroxy-ethyl)-4-((R)-[1-methyl-2-(4-methyl-piperazin-1-yl)-2-oxo-ethyl]-azetidin-2-one (MC-100093)

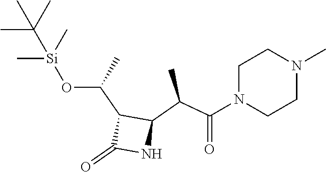 [SMILES: C[C@@H](O[Si](C)(C)C(C)(C)C)[C@H]1C(=O)N[C@@H]1[C@@H](C)C(=O)N1CCN(C)CC1]

Step 1: Synthesis of (3S, 4R)-3-((R)-[1-(tert-butyl-dimethyl-silanyloxy)-ethyl]-4-((R)-[1-methyl-2-(4-methyl-piperazin-1-yl)-2-oxo-ethyl]-azetidin-2-one: To a solution of (R)-2-((2S, 3S)-{3-((R)-[1-(tert-butyl-dimethyl-silanyloxy)-ethyl]-4-oxo-azetidin-2-yl}-propionic acid (10.0 g, 33.17 mmol) in dimethylformamide (200 ml) was added N,N,N′,N′-tetramethyl-O-(7-azabenzotriazol-1-yl)uronium hexafluorophosphate (15.1 g, 39.8 mmol) and diethylisopropylamine (17.2 g, 132.7 mmol). After stirring at room temperature for 30 min, N-methyl piperazine (3.98 g, 39.8 mmol) was added and stirred for 18 h. The solvent was removed under vacuum. The oil remained in the flask was dissolved in 100 ml ethyl acetate and extracted with saturated aqueous NaHCO₃, saturated aqueous NH₄Cl. The organic layer was dried with anhydrous sodium sulfate and filtered. The filtrate was concentrated to oil under reduced pressure. The crude oil was purified by flash chromatography using MeOH/CH₂Cl₂ as eluent afforded (3S, 4R)-3-((R)-[1-(tert-butyl-dimethyl-silanyloxy)-ethyl]-4-((R)-[1-methyl-2-(4-methyl-piperazin-1-yl)-2-oxo-ethyl]-azetidin-2-one as a light-yellow solid. ¹H NMR (400 MHz, DMSO-d6) δ 8.01 (s, 1H), 4.05 (m, 1H), 3.58 (dd, J=2.0 Hz, J=5.3 Hz, 1H), 3.48-3.49 (m, 4H) 2.94 (quint, J=7.0 Hz, 1H), 2.72 (m, 1H), 2.28 (m, 4H), 2.17 (s, 3H), 1.03 (t, J=7.0 Hz, 6H), 0.83 (s, 9H), 0.02 (s, 3H), 0.01 (s, 3H). LC/MS; M+1=384.1

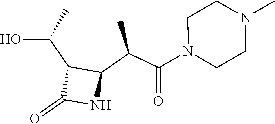 [SMILES: C[C@@H](O)[C@H]1C(=O)N[C@@H]1[C@@H](C)C(=O)N1CCN(C)CC1]

Step 2: Synthesis of (3S, 4R)-3((R)-(1-Hydroxy-ethyl)-4-((R)-[1-methyl-2-(4-methyl-piperazin-1-yl)-2-oxo-ethyl]-azetidin-2-one: To a solution of 3-[1-(tert-butyl-dimethyl-silanyloxy)-ethyl]-4-[1-methyl-2-(4-methyl-piperazin-1-yl)-2-oxo-ethyl]-azetidin-2-one (3.5 g, 9.12 mmol) in 20 mL THF at 0° C. 2 mL HF/Pyridine (70%) was added and stirred for 10 min. The reaction was stirred at room temperature for 18 h. The reaction was cooled in an ice bath and quenched with Conc. NH₄OH to pH 7. The resulting suspension was filtered, and the filtrate was purified on reverse phase 80 g C₁₈ column using H₂O/ACN as eluent afforded (0.940 g, 37%) as off white solid. ¹H NMR (400 MHz, Methanol-d4) δ 4.01 (q, J=6.5 Hz, 1H), 3.73 (dd, J=6.8 Hz, J=2.1 Hz, 1H), 3.63 (m, 4H), 3.04 (q, J=7.1 Hz, 1H), 2.81 (dd, J=6.8, J=2.1 Hz, 1H), 2.49 (m, 2H), 2.42 (t, J=5.1 Hz, 2H) 1.22 (d, J=6.4 Hz, 3H), 1.19 (d, J=6.8 Hz, 3H). LC/MS; M+1=270.

### Example 2: Synthesis of tert-butyl 4-((R)-2-((2R,3S)-3-((R)-1-hydroxyethyl)-4-oxoazetidin-2-yl)propanoyl)piperazine-1-carboxylate

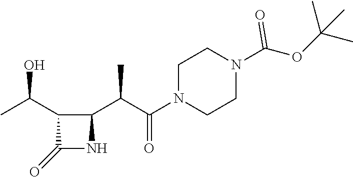 [SMILES: C[C@@H](O)[C@H]1C(=O)N[C@@H]1[C@@H](C)C(=O)N1CCN(C(=O)OC(C)(C)C)CC1]

4-{2-[3-(1-Hydroxy-ethyl)-4-oxo-azetidin-2-yl]-propionyl}-piperazine-1-carboxylic acid tert-butyl ester was prepared by the same procedure of example 1. ¹H NMR (400 MHz, Methanol-d4) δ 4.03 (m, 1H), 3.74 (dd, J=7.4 Hz, J=2.1 Hz, 1H), 3.60 (m, 4H), 3.50 (m, 2H), 3.44 (m, 2H), 3.07 (m, 1H), 2.84 (dd, J=6.8 Hz, J=2.1 Hz, 1H), 1.49 (s, 9H), 1.25 (d, J=6.8 Hz, 3H), 1.21 (d, J=6.9 Hz, 3H). LC/MS; M−56=300.

### Example 3: (3S, 4R)-3-((R)-(1-Hydroxy-ethyl)-4-((R)-(1-methyl-2-oxo-2-piperazin-1-yl-ethyl)-azetidin-2-one

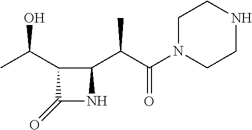 [SMILES: C[C@@H](O)[C@H]1C(=O)N[C@@H]1[C@@H](C)C(=O)N1CCNCC1]

(3S, 4R)-3-((R)-(1-Hydroxy-ethyl)-4-((R)-(1-methyl-2-oxo-2-piperazin-1-yl-ethyl)-azetidin-2-one was prepared by the same procedure of example 1. ¹H NMR (400 MHz, Methanol-d4) δ 4.45 (m, 1H), 3.64 (m, 1H), 3.40 (m, 4H), 3.2 (m, 4H), 2.65 (t, J=10.5 Hz, 1H), 2.5 (m, 1H), 1.33 (s, 3H), 1.31 (s, 3H). LC/MS; M−56=257.

### Example 4: (3S, 4R)-4-((R)-(1-(4-acetylpiperazin-1-yl)-1-oxopropan-2-yl)-3-((R) (1-hydroxyethyl)azetidin-2-one

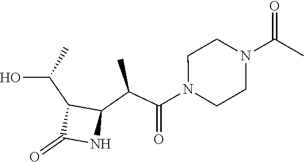 [SMILES: CC(=O)N1CCN(C(=O)[C@H](C)[C@H]2NC(=O)[C@@H]2[C@@H](C)O)CC1]

(3S, 4R)-4-((R)-(1-(4-acetylpiperazin-1-yl)-1-oxopropan-2-yl)-3-((R)-(1-hydroxyethyl)azetidin-2-one was prepared by the same procedure of example 1. ¹H NMR (400 MHz, Methanol-d4) δ 4.03 (pentaplet, J=6.5 Hz, 1H), 3.75 (dd, J=7.4 Hz, J=1.8 Hz, 1H), 3.63 (m, 8H), 3.08 (q, J=7.1 Hz, 1H), 2.86 (m, 1H), 2.15 (s, 3H), 1.24 (d, J=6.3 Hz, 3H), 1.22 (d, J=6.8 Hz, 3H). LC/MS; M+1=297.3

### Example 5: (3S,4R)-4-((R)-1-(4-ethylpiperazin-1-yl)-1-oxopropan-2-yl)-3-((R)-1-hydroxyethyl)azetidin-2-one

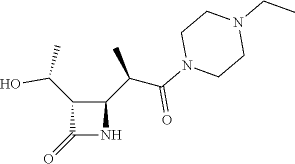 [SMILES: CCN1CCN(C(=O)[C@H](C)[C@H]2NC(=O)[C@@H]2[C@@H](C)O)CC1]

(3S,4R)-4-((R)-1-(4-ethylpiperazin-1-yl)-1-oxopropan-2-yl)-3-((R)-1-hydroxyethyl)azetidin-2-one was prepared by the same procedure of example 1. ¹H NMR (400 MHz, CD₃OD) δ 4.03 (m, 1H), 3.74 (dd, J=2.0 Hz, J=7.4 Hz, 1H), 3.69 (m, 4H), 3.06 (m, 1H), 2.83 (dd, J=2.0 Hz, J=6.9 Hz, 1H), 2.71 (m, 2H), 2.65 (q, J=7.2 Hz, 2H), 2.65 (m, 4H), 1.25 (d, J=6.4 Hz, 3H), 1.21 (d, J=6.9 Hz, 3H), 1.19 (t, J=7.2 Hz, 3H); ESIMS: m/z=284.2 [(M+H)+].

### Example 6: (3S,4R)-3-((R)-1-hydroxyethyl)-4-((R)-1-(4-(methylsulfonyl) piperazin-1-yl)-1-oxopropan-2-yl)azetidin-2-one

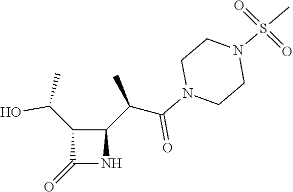 [SMILES: C[C@@H](O)[C@H]1C(=O)N[C@@H]1[C@@H](C)C(=O)N1CCN(S(C)(=O)=O)CC1]

(3S,4R)-3-((R)-1-hydroxyethyl)-4-((R)-1-(4-(methylsulfonyl) piperazin-1-yl)-1-oxopropan-2-yl)azetidin-2-one was prepared by the same procedure of example 1. ¹H NMR (400 MHz, CD₃OD) δ 4.02 (m, 1H), 3.61-3.80 (m, 5H), 3.28 (m, 2H), 3.21 (m, 2H), 3.08 (m, 1H), 2.87 (s, 3H), 2.84 (dd, J=2.1 Hz, J=6.9 Hz, 1H), 1.24 (d, J=6.3 Hz, 3H), 1.21 (d, J=6.9 Hz, 3H); ESIMS: m/z=334.1 [(M+H)+].

### Example 7: (3S,4R)-4-((R)-1-(4-cyclohexylpiperazin-1-yl)-1-oxopropan-2-yl)-3-((R)-1-hydroxyethyl)azetidin-2-one

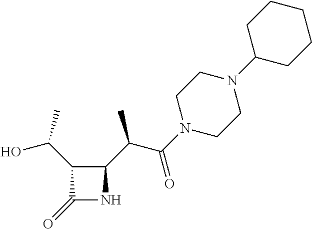 [SMILES: C[C@@H](O)[C@H]1C(=O)N[C@@H]1[C@@H](C)C(=O)N1CCN(C2CCCCC2)CC1]

(3S,4R)-4-((R)-1-(4-cyclohexylpiperazin-1-yl)-1-oxopropan-2-yl)-3-((R)-1-hydroxyethyl)azetidin-2-one as prepared by the same procedure of example 1. ¹H NMR (400 MHz, CD₃OD) δ 4.01 (m, 1H), 3.64-3.85 (m, 5H), 3.06 (m, 1H), 2.99 (m, 2H), 2.94 (m, 2H), 2.83 (dd, J=2.1 Hz, J=7.0 Hz, 1H), 2.77 (m, 1H), 2.02 (m, 2H), 1.90 (m, 2H), 1.70 (m, 1H), 1.36 (m, 5H), 1.24 (d, J=6.3 Hz, 3H), 1.21 (d, J=6.9 Hz, 3H); ESIMS: m/z=338.2 [(M+H)+].

### Example 8: (3S,4R)-4-((R)-1-(4-benzoylpiperazin-1-yl)-1-oxopropan-2-yl)-3-((R)-1-hydroxyethyl)azetidin-2-one

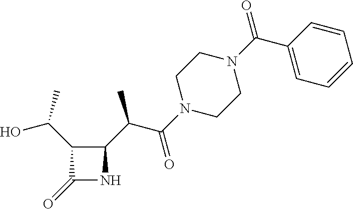 [SMILES: C[C@@H](O)[C@H]1C(=O)N[C@@H]1[C@@H](C)C(=O)N1CCN(C(=O)c2ccccc2)CC1]

(3S,4R)-4-((R)-1-(4-benzoylpiperazin-1-yl)-1-oxopropan-2-yl)-3-((R)-1-hydroxyethyl)azetidin-2-one was prepared by the same procedure of example 1. ¹H NMR (400 MHz, CD₃OD) δ 7.44-7.52 (m, 5H), 4.03 (m, 1H), 3.45-3.90 (m, 9H), 2.97-3.20 (m, 1H), 2.85 (m, 1H), 1.25 (d, J=6.4 Hz, 3H), 1.22 (d, J=6.4 Hz, 3H); ESIMS: m/z=360.2 [(M+H)+]

### Example 9: (3S,4R)-3-((R)-1-hydroxyethyl)-4-((R)-1-oxo-1-(4-phenyl piperazin-1-yl)propan-2-yl)azetidin-2-one

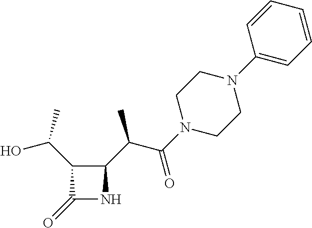 [SMILES: C[C@@H](O)[C@H]1C(=O)N[C@@H]1[C@@H](C)C(=O)N1CCN(c2ccccc2)CC1]

(3S,4R)-3-((R)-1-hydroxyethyl)-4-((R)-1-oxo-1-(4-phenylpiperazin-1-yl) propan-2-yl)azetidin-2-one was prepared by the same procedure of example 1. ¹H NMR (400 MHz, CD₃OD) δ 7.26 (m, 2H), 7.00 (d, J=7.8 Hz, 2H), 6.88 (t, J=7.3 Hz, 1H), 4.04 (m, 1H), 3.77 (m, 5H), 3.21 (q, J=5.4 Hz, J=10.6 Hz, 2H), 3.09-3.16 (m, 3H), 2.85 (dd, J=2.0 Hz, J=6.8 Hz, 1H), 1.25 (d, J=3.4 Hz, 3H), 1.23 (d, J=4.0 Hz, 3H); ESIMS: m/z=332.2 [(M+H)+].

### Example 10: (3S,4R)-3-((R)-1-hydroxyethyl)-4-((R)-1-oxo-1-(4-propyl piperazin-1-yl)propan-2-yl)azetidin-2-one

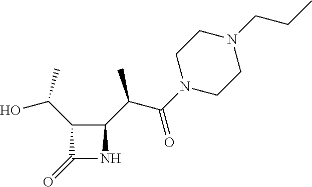 [SMILES: CCCN1CCN(C(=O)[C@H](C)[C@H]2NC(=O)[C@@H]2[C@@H](C)O)CC1]

(3S,4R)-3-((R)-1-hydroxyethyl)-4-((R)-1-oxo-1-(4-propyl piperazin-1-yl)propan-2-yl)azetidin-2-one was prepared by the same procedure of example 1. ¹H NMR (400 MHz, CD₃OD) δ 4.02 (m, 1H), 3.73 (dd, J=2.0 Hz, J=7.5 Hz, 1H), 3.61-3.66 (m, 4H), 3.04 (m, 1H), 2.81 (dd, J=2.1 Hz, J=6.8 Hz, 1H), 2.54 (m, 2H), 2.48 (t, J=5.1 Hz, 2H), 2.38 (m, 2H), 1.56 (m, 2H), 1.23 (d, J=6.3 Hz, 3H), 1.19 (d, J=7.0 Hz, 3H), 0.94 (t, J=7.4 Hz, 3H); ESIMS: m/z=298.2 [(M+H)+]

### Example 11: (3S,4R)-3-((R)-1-hydroxyethyl)-4-((R)-1-(4-(4-methoxyphenyl) piperazin-1-yl)-1-oxopropan-2-yl)azetidin-2-one

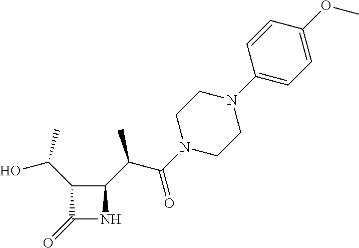 [SMILES: COc1ccc(N2CCN(C(=O)[C@H](C)[C@H]3NC(=O)[C@@H]3[C@@H](C)O)CC2)cc1]

(3S,4R)-3-((R)-1-hydroxyethyl)-4-((R)-1-(4-(4-methoxyphenyl)piperazin-1-yl)-1-oxopropan-2-yl)azetidin-2-one was prepared by the same procedure of example 1: ¹H NMR (400 MHz, CD₃OD) δ 6.97 (m, 2H), 6.85 (m, 2H), 4.03 (m, 1H), 3.73-3.77 (m, 8H), 3.01-3.13 (m, 5H), 2.84 (dd, J=2.1 Hz, J=6.8 Hz, 1H), 1.24 (d, J=5.7 Hz, 3H), 1.22 (d, J=6.2 Hz, 3H); ESIMS: m/z=362.2 [(M+H)+].

### Example 12: (3S,4R)-4-((R)-1-(4-(tert-butyl)piperazin-1-yl)-1-oxopropan-2-yl)-3-((R)-1-hydroxyethyl)azetidin-2-one

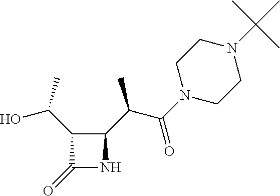 [SMILES: C[C@@H](O)[C@H]1C(=O)N[C@@H]1[C@@H](C)C(=O)N1CCN(C(C)(C)C)CC1]

(3S,4R)-4-((R)-1-(4-(tert-butyl)piperazin-1-yl)-1-oxopropan-2-yl)-3-((R)-1-hydroxyethyl)azetidin-2-one was prepared by the same procedure of example 1: ¹H NMR (400 MHz, CD₃OD) δ 4.03 (m, 1H), 3.74 (dd, J=2.0 Hz, J=7.5 Hz, 1H), 3.57-3.66 (m, 4H), 3.05 (m 1H), 2.82 (dd, J=2.1 Hz, J=6.8 Hz, 1H), 2.69 (m, 2H), 2.62 (t, J=5.2 Hz, 2H), 1.24 (d, J=6.4 Hz, 3H), 1.20 (d, J=6.9 Hz, 3H), 1.13 (s, 9H); ESIMS: m/z=312.2 [(M+H)+].

### Example 13: 4-((R)-2-((2R,3S)-3-((R)-1-hydroxyethyl)-4-oxoazetidin-2-yl)propanoyl)piperazine-1-carboxamide

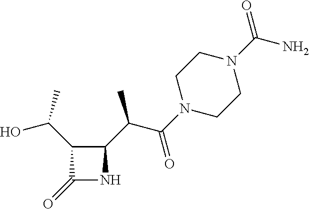 [SMILES: C[C@@H](O)[C@H]1C(=O)N[C@@H]1[C@@H](C)C(=O)N1CCN(C(N)=O)CC1]

4-((R)-2-((2R,3S)-3-((R)-1-hydroxyethyl)-4-oxoazetidin-2-yl)propanoyl) piperazine-1-carboxamide was prepared by the same procedure of example 1: ¹H NMR (400 MHz, CD₃OD) δ 4.02 (m, 1H), 3.74 (dd, J=2.0 Hz, J=7.4 Hz, 1H), 3.57-3.66 (m, 4H), 3.49 (m, 2H), 3.41 (m, 2H), 3.07 (m 1H), 2.84 (dd, J=2.1 Hz, J=6.8 Hz, 1H), 1.23 (d, J=6.4 Hz, 3H), 1.20 (d, J=6.9 Hz, 3H); ESIMS: m/z=299.2 [(M+H)+].

### Example 14: (3S,4R)-3-((R)-1-hydroxyethyl)-4-((R)-1-(4-methyl-3,4-dihydro quinoxalin-1(2H)-yl)-1-oxopropan-2-yl)azetidin-2-one

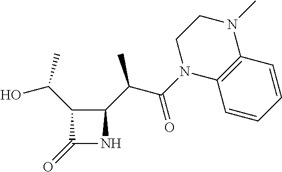 [SMILES: C[C@@H](O)[C@H]1C(=O)N[C@@H]1[C@@H](C)C(=O)N1CCN(C)c2ccccc21]

(3S,4R)-3-((R)-1-hydroxyethyl)-4-((R)-1-(4-methyl-3,4-dihydro quinoxalin-1(2H)-yl)-1-oxopropan-2-yl)azetidin-2-one was prepared by the same procedure of example 1: ¹H NMR (400 MHz, CDCl₃) δ 7.16 (t, J=7.4 Hz, 1H), 6.94 (d, J=7.7 Hz, 1H), 6.72 (d, J=8.0 Hz, 1H), 6.67 (t, J=7.6 Hz, 1H), 5.91 (s, 1H), 3.99-4.16 (m, 2H), 3.74 (m, 1H), 3.61 (m, 1H), 3.45-3.51 (m 1H), 3.32 (m, 1H), 3.22 (m, 1H), 3.10 (s, 1H), 2.96 (s, 3H), 2.61 (d, J=Hz, 1H), 1.28 (d, J=6.2 Hz, 3H), 1.17 (d, J=6.8 Hz, 3H); ESIMS: m/z=318.1 [(M+H)+]

### Example 15: (R)-1-((2R,3S)-2-((R)-1-(4-methylpiperazin-1-yl)-1-oxopropan-2-yl)-4-oxoazetidin-3-yl)ethyl acetate

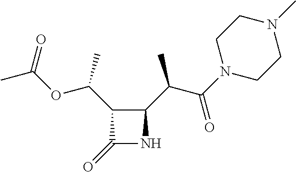 [SMILES: CC(=O)O[C@H](C)[C@H]1C(=O)N[C@@H]1[C@@H](C)C(=O)N1CCN(C)CC1]

Step 1: Synthesis of (R)-benzyl 2-((2S,3S)-3-((R)-1-acetoxyethyl)-4-oxoazetidin-2-yl)propanoate: Acetyl chloride (1.623 mmole) was added to a solution of (R)-benzyl 2-((2S,3S)-3-((R)-1-hydroxyethyl)-4-oxoazetidin-2-yl)propanoate (300 mg; 1.082 mmole) in dichloromethane (6.0 ml) and pyridine (348 uL; 4.328 mmole). This mixture was stirred at room temperature overnight. The reaction solution was diluted with dichloromethane and washed with water (2×), 1N aqueous HCl (2×), water, saturated sodium bicarbonate and brine. The organic solution was dried over anhydrous sodium sulfate, filtered and concentrated. The crude product was purified by column chromatography on silica gel using a 0 to 10% methanol in dichloromethane gradient solvent system. Pure desired (R)-benzyl 2-((2S,3S)-3-((R)-1-acetoxyethyl)-4-oxoazetidin-2-yl)propanoate was obtained as a colorless oil. 1H NMR (400 MHz, CDCl₃) δ 7.32-7.41 (m, 5H), 5.92 (s, 1H), 5.20 (m, 1H), 5.14 (s, 2H), 3.76 (dd, J=2.2 Hz, J=6.2 Hz, 1H), 3.16 (dd, J=2.1 Hz, J=7.7 Hz, 1H), 2.72 (m, 1H), 2.03 (s, 3H), 1.32 (d, J=6.3 Hz, 3H), 1.24 (d, J=7.0 Hz, 3H); ESIMS: m/z=320.1 [(M+H)+].

Step 2: Synthesis of (R)-2-((2S,3S)-3-((R)-1-acetoxyethyl)-4-oxoazetidin-2-yl)propanoic acid: (R)-benzyl 2-((2S,3S)-3-((R)-1-acetoxyethyl)-4-oxoazetidin-2-yl)propanoate was dissolved into methanol (4.0 mL). 10% palladium on carbon (20 mg) was added and this mixture was stirred under hydrogen (balloon pressure) at room temperature for 3 hours. The reaction solution was filtered through celite and concentrated down to yield 13b (58 mg; 85% yield) as a pale yellow oil. ESIMS: m/z=230.1 [(M+H)+].

Step 3: Synthesis of (R)-1-((2R,3S)-2-((R)-1-(4-methylpiperazin-1-yl)-1-oxopropan-2-yl)-4-oxoazetidin-3-yl)ethyl acetate: 4-(4,6-dimethoxy-1,3,5-triazin-2-yl)-4-methyl morpholinium chloride (14.0 mg; 0.0506 mmole) and 1-methyl piperazine (5.6 uL; 0.0506 mmole) were added sequentially to a solution of (R)-2-((2S,3S)-3-((R)-1-acetoxyethyl)-4-oxoazetidin-2-yl)propanoic acid (11.60 mg; 0.0506 mmole) in tetrahydrofuran (1.0 mL). The resulting suspension was stirred at room temperature overnight. The tan solid was filtered off and the filtrate was concentrated down. This crude product was purified by column chromatography on silica gel using a 0 to 20% methanol in dichloromethane gradient solvent system. The title compound was obtained as a colorless oil. 1H NMR (400 MHz, CD₃OD) δ 5.15 (m, 1H), 3.62-3.72 (m, 5H), 3.03-3.08 (m, 2H), 2.46-2.53 (m, 4H), 2.35 (s, 3H), 2.02 (s, 3H), 1.29 (d, J=6.4 Hz, 3H), 1.17 (d, J=6.8 Hz, 3H); ESIMS: m/z=312.2 [(M+H)+].

### Example 16: (R)-1-((2R,3S)-2-((R)-1-(4-methylpiperazin-1-yl)-1-oxopropan-2-yl)-4-oxoazetidin-3-yl)ethyl propionate

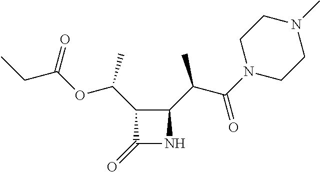 [SMILES: CCC(=O)O[C@H](C)[C@H]1C(=O)N[C@@H]1[C@@H](C)C(=O)N1CCN(C)CC1]

(R)-1-((2R,3S)-2-((R)-1-(4-methylpiperazin-1-yl)-1-oxopropan-2-yl)-4-oxoazetidin-3-yl)ethyl propionate was prepared by the same procedure of example 15: ¹H NMR (400 MHz, CD₃OD) δ 5.17 (m, 1H), 3.74 (dd, J=2.1 Hz, J=7.8 Hz, 1H), 3.63-3.71 (m, 4H), 3.06 (m, 2H), 2.49-2.56 (m, 4H), 2.36 (s, 3H), 2.33 (m, 2H), 1.29 (d, J=6.4 Hz, 3H), 1.18 (d, J=6.8 Hz, 3H), 1.11 (t, J=7.5 Hz, 3H); ESIMS: m/z=326.2 [(M+H)+].

### Example 17: (R)-1-((2R,3S)-2-((R)-1-(4-methylpiperazin-1-yl)-1-oxopropan-2-yl)-4-oxoazetidin-3-yl)ethyl butyrate

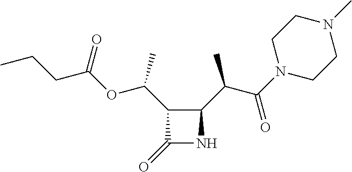 [SMILES: CCCC(=O)O[C@H](C)[C@H]1C(=O)N[C@@H]1[C@@H](C)C(=O)N1CCN(C)CC1]

(R)-1-((2R,3S)-2-((R)-1-(4-methylpiperazin-1-yl)-1-oxopropan-2-yl)-4-oxoazetidin-3-yl)ethyl butyrate was prepared by the same procedure of example 15: ¹H NMR (400 MHz, CD₃OD) δ 5.18 (m, 1H), 3.74 (dd, J=2.1 Hz, J=7.7 Hz, 1H), 3.63-3.71 (m, 4H), 3.06 (m, 2H), 2.48-2.56 (m, 4H), 2.37 (s, 3H), 2.27-2.32 (m, 2H), 1.64 (m, 2H), 1.29 (d, J=6.4 Hz, 3H), 1.18 (d, J=6.8 Hz, 3H), 0.95 (t, J=7.4 Hz, 3H); ESIMS: m/z=340.2 [(M+H)+]

### Example 18: (R)-1-((2R,3S)-2-((R)-1-(4-methylpiperazin-1-yl)-1-oxopropan-2-yl)-4-oxoazetidin-3-yl)ethyl isobutyrate

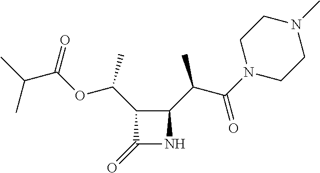 [SMILES: CC(C)C(=O)O[C@H](C)[C@H]1C(=O)N[C@@H]1[C@@H](C)C(=O)N1CCN(C)CC1]

(R)-1-((2R,3S)-2-((R)-1-(4-methylpiperazin-1-yl)-1-oxopropan-2-yl)-4-oxoazetidin-3-yl)ethyl isobutyrate was prepared by the same procedure of example 15: 1H NMR (400 MHz, CD₃OD) δ 5.17 (m, 1H), 3.79 (dd, J=2.2 Hz, J=7.6 Hz, 1H), 3.64-3.69 (m, 4H), 3.08 (m, 2H), 2.45-2.57 (m, 5H), 2.37 (s, 3H), 1.27 (d, J=6.4 Hz, 3H), 1.19 (d, J=6.8 Hz, 3H), 1.16 (t, J=5.6 Hz, 3H); ESIMS: m/z=340.2 [(M+H)+].

### Example 19: (R)-1-((2R,3S)-2-((R)-1-(4-methylpiperazin-1-yl)-1-oxopropan-2-yl)-4-oxoazetidin-3-yl)ethyl pivalate

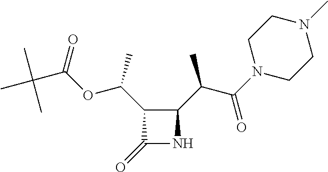 [SMILES: C[C@@H](OC(=O)C(C)(C)C)[C@H]1C(=O)N[C@@H]1[C@@H](C)C(=O)N1CCN(C)CC1]

(R)-1-((2R,3S)-2-((R)-1-(4-methylpiperazin-1-yl)-1-oxopropan-2-yl)-4-oxoazetidin-3-yl)ethyl pivalate was prepared by the same procedure of example 15: ¹H NMR (400 MHz, CD₃OD) δ 5.15 (m, 1H), 3.85 (dd, J=2.1 Hz, J=7.4 Hz, 1H), 3.63-3.69 (m, 4H), 3.10 (m, 2H), 2.46-2.53 (m, 4H), 2.36 (s, 3H), 1.26 (d, J=6.5 Hz, 3H), 1.18-1.21 (m, 12H); ESIMS: m/z=354.2 [(M+H)+].

### FORMULATIONS

The present disclosure also relates to compositions or formulations which comprise the beta lactams according to the present disclosure. In general, the compositions of the present disclosure comprise an effective amount of one or more beta lactams or salts thereof according to the present disclosure which are effective for providing treatment or prevention of alcohol dependence and/or alcohol associated fatty liver diseases; and one or more excipients.

For the purposes of the present disclosure, the term “excipient” and “carrier” are used interchangeably throughout the description of the present disclosure and said terms are defined herein as, “ingredients which are used in the practice of formulating a safe and effective pharmaceutical composition.”

The formulator will understand that excipients are used primarily to serve in delivering a safe, stable, and functional pharmaceutical, serving not only as part of the overall vehicle for delivery but also as a means for achieving effective absorption by the recipient of the active ingredient. An excipient may fill a role as simple and direct as being an inert filler, or an excipient as used herein may be part of a pH stabilizing system or coating to insure delivery of the ingredients safely to the stomach. The formulator can also take advantage of the fact the compounds of the present disclosure have improved cellular potency, pharmacokinetic properties, as well as improved oral bioavailability.

The present disclosure also provides pharmaceutical compositions that include at least one compound described herein and one or more pharmaceutically acceptable carriers, excipients, or diluents. Examples of such carriers are well known to those skilled in the art and can be prepared in accordance with acceptable pharmaceutical procedures, such as, for example, those described in Remington's Pharmaceutical Sciences, 17th edition, ed. Alfonoso R. Gennaro, Mack Publishing Company, Easton, PA (1985), the entire disclosure of which is incorporated by reference herein for all purposes. As used herein, “pharmaceutically acceptable” refers to a substance that is acceptable for use in pharmaceutical applications from a toxicological perspective and does not adversely interact with the active ingredient. Accordingly, pharmaceutically acceptable carriers are those that are compatible with the other ingredients in the formulation and are biologically acceptable. Supplementary active ingredients can also be incorporated into the pharmaceutical compositions.

Compounds of the present disclosure can be administered orally or parenterally, neat or in combination with conventional pharmaceutical carriers. Applicable solid carriers can include one or more substances which can also act as flavoring agents, lubricants, solubilizers, suspending agents, fillers, glidants, compression aids, binders or tablet-disintegrating agents, or encapsulating materials. The compounds can be formulated in conventional manner, for example, in a manner similar to that used for known beta lactams. Oral formulations containing a compound disclosed herein can comprise any conventionally used oral form, including tablets, capsules, buccal forms, troches, lozenges and oral liquids, suspensions or solutions. In powders, the carrier can be a finely divided solid, which is an admixture with a finely divided compound. In tablets, a compound disclosed herein can be mixed with a carrier having the necessary compression properties in suitable proportions and compacted in the shape and size desired. The powders and tablets can contain up to 99% of the compound.

Capsules can contain mixtures of one or more compound(s) disclosed herein with inert filler(s) and/or diluent(s) such as pharmaceutically acceptable starches (e.g., corn, potato or tapioca starch), sugars, artificial sweetening agents, powdered celluloses (e.g., crystalline and microcrystalline celluloses), flours, gelatins, gums, and the like.

Useful tablet formulations can be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins. Surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine. Oral formulations herein can utilize standard delay or time-release formulations to alter the absorption of the compound(s). The oral formulation can also consist of administering a compound disclosed herein in water or fruit juice, containing appropriate solubilizers or emulsifiers as needed.

Liquid carriers can be used in preparing solutions, suspensions, emulsions, syrups, elixirs, and for inhaled delivery. A compound of the present disclosure can be dissolved or suspended in a pharmaceutically acceptable liquid carrier such as water, an organic solvent, or a mixture of both, or a pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, and osmo-regulators. Examples of liquid carriers for oral and parenteral administration include, but are not limited to, water (particularly containing additives as described herein, e.g., cellulose derivatives such as a sodium carboxymethyl cellulose solution), alcohols (including monohydric alcohols and polyhydric alcohols, e.g., glycols) and their derivatives, and oils (e.g., fractionated coconut oil and arachis oil). For parenteral administration, the carrier can be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellants.

Liquid pharmaceutical compositions, which are sterile solutions or suspensions, can be utilized by, for example, intramuscular, intraperitoneal (“i.p.”) or subcutaneous injection. Sterile solutions can also be administered intravenously. Compositions for oral administration can be in either liquid or solid form.

Preferably the pharmaceutical composition is in unit dosage form, for example, as tablets, capsules, powders, solutions, suspensions, emulsions, granules, or suppositories. In such form, the pharmaceutical composition can be sub-divided in unit dose(s) containing appropriate quantities of the compound. The unit dosage forms can be packaged compositions, for example, packeted powders, vials, ampoules, prefilled syringes, or sachets containing liquids. Alternatively, the unit dosage form can be a capsule or tablet itself, or it can be the appropriate number of any such compositions in package form. Such unit dosage form can contain from about 1 mg/kg of compound to about 500 mg/kg of compound, and can be given in a single dose or in two or more doses. Such doses can be administered in any manner useful in directing the compound(s) to the recipient's bloodstream, including orally, via implants, parenterally (including intravenous, intraperitoneal and subcutaneous injections), rectally, vaginally, and transdermally.

When administered for the treatment or inhibition of a particular disease state or disorder, it is understood that an effective dosage can vary depending upon the particular compound utilized, the mode of administration, and severity of the condition being treated, as well as the various physical factors related to the individual being treated. In therapeutic applications, a compound of the present disclosure can be provided to a patient already suffering from a disease in an amount sufficient to cure or at least partially ameliorate the symptoms of the disease and its complications. The dosage to be used in the treatment of a specific individual typically must be subjectively determined by the attending physician. The variables involved include the specific condition and its state as well as the size, age and response pattern of the patient.

In some cases, it may be desirable to administer a compound directly to the airways of the patient, using devices such as, but not limited to, metered dose inhalers, breath-operated inhalers, multidose dry-powder inhalers, pumps, squeeze-actuated nebulized spray dispensers, aerosol dispensers, and aerosol nebulizers. For administration by intranasal or intrabronchial inhalation, the compounds of the present disclosure can be formulated into a liquid composition, a solid composition, or an aerosol composition. The liquid composition can include, by way of illustration, one or more compounds of the present disclosure dissolved, partially dissolved, or suspended in one or more pharmaceutically acceptable solvents and can be administered by, for example, a pump or a squeeze-actuated nebulized spray dispenser. The solvents can be, for example, isotonic saline or bacteriostatic water. The solid composition can be, by way of illustration, a powder preparation including one or more compounds of the present disclosure intermixed with lactose or other inert powders that are acceptable for intrabronchial use, and can be administered by, for example, an aerosol dispenser or a device that breaks or punctures a capsule encasing the solid composition and delivers the solid composition for inhalation. The aerosol composition can include, by way of illustration, one or more compounds of the present disclosure, propellants, surfactants, and co-solvents, and can be administered by, for example, a metered device. The propellants can be a chlorofluorocarbon (CFC), a hydrofluoroalkane (HFA), or other propellants that are physiologically and environmentally acceptable.

Compounds described herein can be administered parenterally or intraperitoneally. Solutions or suspensions of these compounds or a pharmaceutically acceptable salts, hydrates, or esters thereof can be prepared in water suitably mixed with a surfactant such as hydroxyl-propylcellulose. Dispersions can also be prepared in glycerol, liquid polyethylene glycols, and mixtures thereof in oils. Under ordinary conditions of storage and use, these preparations typically contain a preservative to inhibit the growth of microorganisms.

The pharmaceutical forms suitable for injection can include sterile aqueous solutions or dispersions and sterile powders for the extemporaneous preparation of sterile injectable solutions or dispersions. In some embodiments, the form can sterile, and its viscosity permits it to flow through a syringe. The form preferably is stable under the conditions of manufacture and storage and can be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Compounds described herein can be administered transdermally, i.e., administered across the surface of the body and the inner linings of bodily passages including epithelial and mucosal tissues. Such administration can be carried out using the compounds of the present disclosure including pharmaceutically acceptable salts, hydrates, or esters thereof, in lotions, creams, foams, patches, suspensions, solutions, and suppositories (rectal and vaginal).

Transdermal administration can be accomplished through the use of a transdermal patch containing a compound, such as a compound disclosed herein, and a carrier that can be inert to the compound, can be non-toxic to the skin, and can allow delivery of the compound for systemic absorption into the blood stream via the skin. The carrier can take any number of forms such as creams and ointments, pastes, gels, and occlusive devices. The creams and ointments can be viscous liquid or semisolid emulsions of either the oil-in-water or water-in-oil type. Pastes comprised of absorptive powders dispersed in petroleum or hydrophilic petroleum containing the compound can also be suitable. A variety of occlusive devices can be used to release the compound into the blood stream, such as a semi-permeable membrane covering a reservoir containing the compound with or without a carrier, or a matrix containing the compound. Other occlusive devices are known in the literature.

Compounds described herein can be administered rectally or vaginally in the form of a conventional suppository. Suppository formulations can be made from traditional materials, including cocoa butter, with or without the addition of waxes to alter the suppository's melting point, and glycerin. Water-soluble suppository bases, such as polyethylene glycols of various molecular weights, can also be used.

Lipid formulations or nanocapsules can be used to introduce compounds of the present disclosure into host cells either in vitro or in vivo. Lipid formulations and nanocapsules can be prepared by methods known in the art.

To increase the effectiveness of compounds of the present disclosure, it can be desirable to combine a compound with other agents effective in the treatment of the target disease. For example, other active compounds (i.e., other active ingredients or agents) effective in treating the target disease can be administered with compounds of the present disclosure. The other agents can be administered at the same time or at different times than the compounds disclosed herein.

Compounds of the present disclosure can be useful for the treatment or inhibition of a pathological condition or disorder in a mammal, for example, a human subject. The present disclosure accordingly provides methods of treating or inhibiting a pathological condition or disorder by providing to a mammal a compound of the present disclosure including its pharmaceutically acceptable salt) or a pharmaceutical composition that includes one or more compounds of the present disclosure in combination or association with pharmaceutically acceptable carriers. Compounds of the present disclosure can be administered alone or in combination with other therapeutically effective compounds or therapies for the treatment or inhibition of the pathological condition or disorder.

Non-limiting examples of compositions according to the present disclosure include from about 0.001 mg to about 1,000 mg of one or more beta lactams according to the present disclosure and one or more excipients; from about 0.01 mg to about 100 mg of one or more beta lactams according to the present disclosure and one or more excipients; and from about 0.1 mg to about 10 mg of one or more beta lactams according to the present disclosure; and one or more excipients.

The present disclosure also provides a method for preventing, treating, or lessen effects of, alcohol dependence. Such as method comprises administering to a subject an effective amount of a compound or composition according to the present disclosure. Such a method includes administering to a subject in need thereof a composition comprising an effective amount of one or more compounds according to the present disclosure and at least one excipient.

In some embodiments, the alcohol dependence is treated or prevented by attenuating alteration in glutamate transporters and/or reducing alcohol associated neuroinflammation.

The present disclosure also provides a method for preventing, treating, or lessen effects of alteration in glutamate transporters in the subject associated with alcohol uses. In some embodiments, the method is for attenuating alteration in glutamate transporters in the subject. Such as method comprises administering to a subject an effective amount of a compound or composition according to the present disclosure. Such a method includes administering to a subject in need thereof a composition comprising an effective amount of one or more compounds according to the present disclosure and at least one excipient.

The present disclosure also provides a method for reducing alcohol associated neuroinflammation in the subject. Such as method comprises administering to a subject an effective amount of a compound or composition according to the present disclosure. Such a method includes administering to a subject in need thereof a composition comprising an effective amount of one or more compounds according to the present disclosure and at least one excipient.

The present disclosure also provides a method for preventing, treating, or lessen effects of, alcohol-associated fatty liver diseases. Such as method comprises administering to a subject an effective amount of a compound or composition according to the present disclosure. Such a method includes administering to a subject in need thereof a composition comprising an effective amount of one or more compounds according to the present disclosure and at least one excipient.

In some embodiments, the composition is administrated orally or through injection. The effective amount is a dose of the compound may be in a range of from 0.001 mg/Kg of the subject to 200 mg/Kg of the subject. For example, the dose of the compound is in a range of from 1 mg/Kg of the subject to 100 mg/Kg of the subject.

In the methods described herein, in some embodiments, the compound is (3S, 4R)-3-((R)-(1-hydroxy-ethyl)-4-((R)-[1-methyl-2-(4-methyl-piperazin-1-yl)-2-oxo-ethyl]-azetidin-2-one (MC-100093), or a pharmaceutically acceptable salt or complex thereof. The pharmaceutical composition further comprises at least one excipient. The composition is administrated through injection in some embodiments.

In some embodiments, the subject is a human subject. The administration is made through rejection.

### Efficacy Evaluation

### A. Alcohol Dependence and Alcohol Associated Liver Diseases

The following procedures can be utilized in evaluating and selecting compounds and compositions for treating, lessening effect of, or preventing alcohol dependence, and/or alcohol associated liver diseases. The descriptions are based on exemplary compound MC-100093.

### A-1. Materials and Methods

### Animals:

P rats were received from Indiana University School of Medicine (Indianapolis, IN, USA), and were housed in a 21° C. on a 12/12 h light/dark cycle-maintained room where they had free access to water and food. All experimental procedures were approved by the Institutional Animal Care and Use Committee (IACUC) of The University of Toledo, in accordance with the guidelines governing the use of animals in research of the National Institutes of Health as described in the Guidefor the Care and Use of Laboratory Animals.

### Ethanol Drinking Protocol:

We used an ethanol drinking paradigm as described previously by Alhaddad, et al. (2014). Briefly, animals started the experimental drinking procedure at age of 90 days. The animals had five weeks of exposure to continuous three-bottle choice drinking, 0%, 15% and 30%, v/v ethanol in water, available concurrently. Daily measurement of ethanol intake was performed (g of ethanol intake/kg of body weight/day). We measured the baseline ethanol drinking by average the intake during the last three weeks. Three groups of P rats (n=8-9/group) exposed to the five weeks ethanol exposure. At week 6, rats in each group received daily intraperitoneal injection of either MC-100093 at dose of 50 mg/kg (Ethanol-MC group), Ceftriaxone at dose of 200 mg/kg (Ethanol-CEF group, which also serves as positive control), or equivolume of saline vehicle (Ethanol group) for five days. This paradigm results in pharmacologically relevant blood alcohol concentration (50-200 mg %). Following the criteria for the development of ethanol dependence, rats whose average ethanol intake <4 g/kg/day were excluded from the study. Beside the ethanol exposed groups, another group of P rats (n=8) exposed to only water and food throughout the exposure procedures (control group).

Ceftriaxone was used as positive control since it has the beta lactam structure. The compounds used in the present disclosure, such as MC-100093, which are a beta lactam compound without any antibiotic action as compared to ceftriaxone, which has it.

### Brain Tissue Dissection:

We used micro-punch procedure to dissect the subregions of the NAc such as NAc-core and NAc-shell as well as the subregions of prefrontal cortex such as infralimbic (IL) and prelimbic (PL) from the brain on day 6 after the last injection day. P rats were removed from the cage and rapidly euthanized by CO₂ inhalation followed by rapid decapitation with a guillotine. Brains were isolated and immediately frozen on dry ice and stored at −80° C. The stereotaxic coordinates provided by Paxinos and colleague's rat brain stereotactic atlas was used to isolate NAc-core and NAc-shell using a cryostat apparatus (−20° C.), which then kept at −80° C. for later western blot analyses.

### Western Blot Analyses:

Western Blot assay was performed to measure changes in the expression of GLT-1, xCT, mGluR1, mGluR5, PPAR-α, PPARγ, GAPDH and β-tubulin in the NAc-core and NAc-shell as described in previous publications. A lysis buffer containing protease and phosphatase inhibitors was used to generate a homogenate of liver, Nac-core, and Nac-shell samples. The amount of protein was quantified using a detergent compatible protein assay (Bio-Rad, Hercules, CA, USA) for each sample. Samples were mixed with laemmli dye and loaded with equal amounts on polyacrylamide gels (10%), which separated the proteins using electrophoresis. After that, polyvinylidene difluoride (PVDF) membranes were used to transfer the proteins electrophoretically from the gels. Membranes were incubated in 5% free-fat milk in Tris-buffered saline with Tween-20 (TBST) for 30-60 minutes at room temperature. Membranes were then incubated with appropriate primary antibodies overnight at 4° C.: rabbit anti-GLT-1 (1:5000, Abcam, ab41621), rabbit anti-xCT (1:2000, Abcam, ab175186), rabbit anti-mGluR1 (1:1000, Abcam, ab82211), rabbit anti-mGluR5 (1:1000, Abcam, ab76316), rabbit anti-PPAR-α (1:1000, Abcam, ab24509), and rabbit anti-PPARγ (1:1000, Abcam, ab209350). Mouse anti-β-tubulin (1:1000; BioLegend) was used as a loading control antibody for the brain samples and rabbit anti-GAPDH (1:5000; cell signaling) was used as a loading control antibody for the liver samples. Membranes were washed five times on the next day with TBST followed by incubation with appropriate secondary antibody (1:5000) for 90 minutes at room temperature. Chemiluminescent reagents (Super Signal West Pico, Pierce Inc.) were used to detect the proteins using a GeneSys imaging system to develop the digitized blot images. Water-control group data were represented as 100% and all other values were expressed relative to control group to detect changes in the expression of the protein of interest in the brain and liver samples.

### Liver Oil Red O Staining:

To measure the fat content in the liver sections, we used Oil Red O (Sigma, CAS Number 1320-06-5) staining on 10 μm thick formalin-fixed liver sections and placed in slides for further staining. The slides were stained with freshly prepared Oil Red O working solution 15 minutes then rinsed with 60% isopropanol. Slides were then rinsed with distilled water and mounted in aqueous mounting media and prepared for imaging. The degree of Oil Red O staining was determined at 20× magnification using a color video camera attached to an Olympus VS120 slide scanning microscope. Olympus OlyVIA software was used to analyze the images. The lipid droplets were quantified using Image J pro (NIH). Data are presented as the mean+SEM of the Oil Red O staining for each group.

### Statistical Analysis:

All statistical analyses were conducted with Graphpad Prism software, with p-values of 0.05 or less being statistically significant. We performed a two-way (mixed model) ANOVA followed by Bonferroni multiple comparison post-hoc test to analyze 24 h ethanol consumption, 24 h water consumption, and daily body weight. For the Western blot and oil red O staining analysis, we performed one-way ANOVA with Newman-Keuls as a post hoc multiple comparison test to measure differences between groups as a percentage (relative to control values).

### A-2. Results

(1) Effect of MC-100093 (50 mg/kg, i.p.) and Ceftriaxone (200 mg/kg, i.p.) Treatment on Ethanol Consumption:

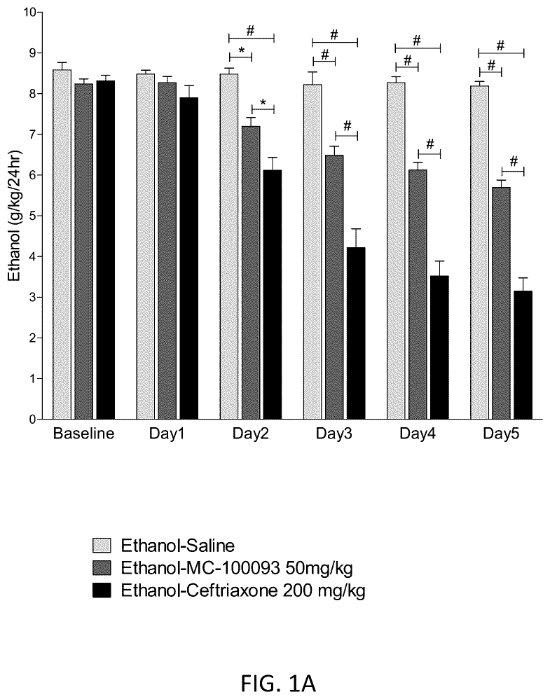

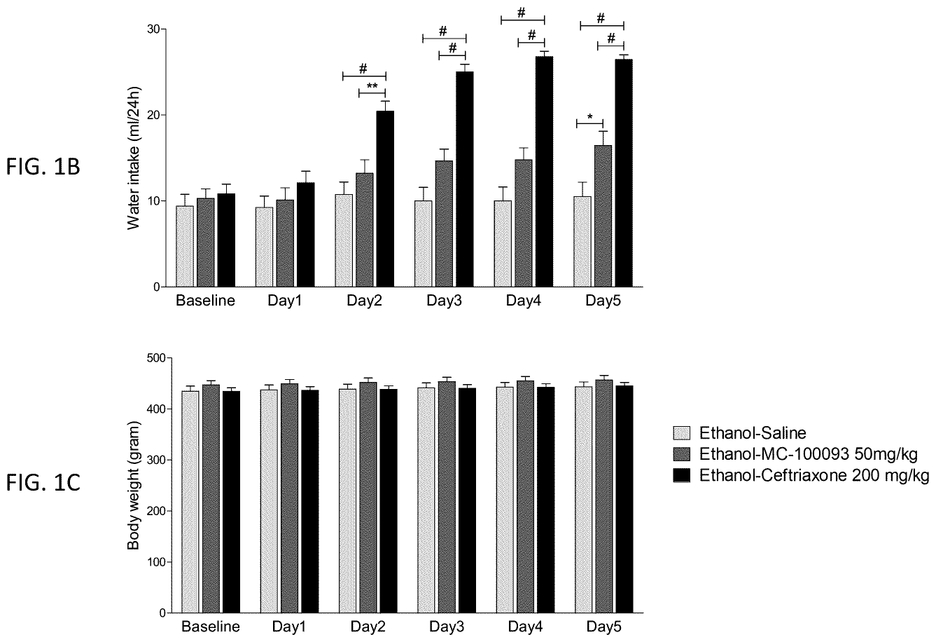

Treatment with MC-100093 or Ceftriaxone significantly reduced P rat's ethanol consumption compared to saline treatment. Statistical analysis of the ethanol drinking data revealed a significant Dose by Day interaction (F10,115=29.35, p<0.0001). Bonferroni multiple comparison test showed a significant decrease in ethanol consumption from day 1 through day 5 in the MC-100093 (50 mg/kg, i.p.) and ceftriaxone (200 mg/kg, i.p.) groups compared to the Ethanol-Saline group (FIG. 1 A).

### (2) Effects of MC-100093 and Ceftriaxone Treatment on Water Intake:

The water consumption was significantly increased in MC-100093 or Ceftriaxone treated groups compared to saline treated group. Statistical analysis of water consumption data revealed a significant Dose by Day interaction (F10,115=25.13, p<0.0001). Bonferroni multiple comparison tests showed a significant increase in water consumption from day 1 through day 5 in ceftriaxone (200 mg/kg, i.p.) group and at day 5 in the MC-100093 (50 mg/kg, i.p.) group compared to the Ethanol-Saline group (FIG. 1 B).

(3) Effects of MC-100093 (50 mg/kg, i.p.) and Ceftriaxone (200 mg/kg, i.p.) Treatment on Body Weight:

Statistical analysis of body weight data revealed a non-significant Dose by Day interaction (F_{10,115}=0.98, p<0.0001). Neither MC-100093 (50 mg/kg, i.p.) nor Ceftriaxone (200 mg/kg, i.p.) treatment has significant effect on animal's body weight (FIG. 1 C).

As shown in FIGS. 1A-1C, treatment with MC-100093 (50 mg/kg, i.p.) or ceftriaxone (200 mg/kg, i.p.) significantly reduced ethanol consumption from day 2 through day 5, with a concomitant significant increase in water consumption from day 2 through day 5 for Ethanol-Ceftriaxone and at day 5 with Ethanol-MC when compared to the Ethanol-Saline group. However, ethanol consumption was significantly lower and water consumption was significantly higher in Ethanol-Ceftriaxone compared to Ethanol-MC group on days 2 through day 5. There were no significant effects of MC-100093 (50 mg/kg, i.p.) or ceftriaxone (200 mg/kg, i.p.) treatment on body weight. The values are expressed as mean±SEM (n=8/group for Ethanol-Saline and n=9/group for Ethanol-MC and Ethanol-Ceftriaxone groups), (*p<0.05 and **p<0.01, and #p<0.0001).

(4) Effect of MC-100093 (50 mg/kg, i.p.) and Ceftriaxone (200 mg/kg, i.p.) on the Expression of GLT-1 in the NAc-Core and NAc-Shell:

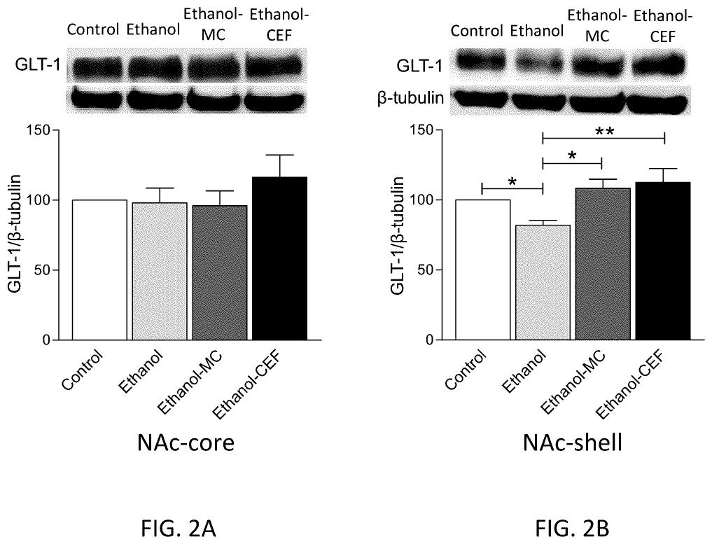

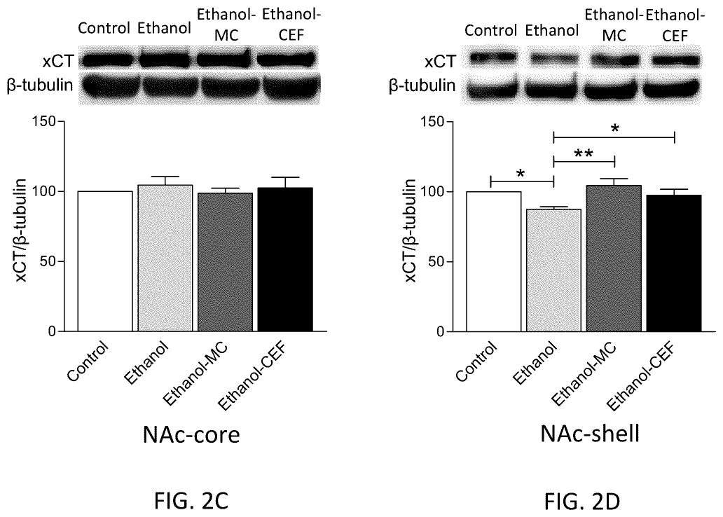

FIG. 2A-2B show the effects of MC-100093 (50 mg/kg, i.p.) or ceftriaxone (200 mg/kg, i.p.) treatment on: (A) Protein expression of GLT-1 in the NAc-core, and (B) Protein expression of GLT-1 in the NAc-shell. In FIG. 2A, the upper panel shows representative immunoblot of GLT-1 and β-tubulin in the NAc-core, and in the lower panel, quantitative analysis revealed a non-significant difference in the expression of GLT-1 among Control, Ethanol, Ethanol-MC (50 mg/kg), and Ethanol-CEF (200 mg/kg) groups in the NAc-core. In FIG. 2B, the upper panel shows representative immunoblot of GLT-1 and β-tubulin in the NAc-shell, and in the lower panel, quantitative analysis revealed a significant downregulation in the expression of GLT-1 in the Ethanol group compared to the control group.

We investigated the effects of MC-100093 (50 mg/kg, i.p.) or Ceftriaxone (200 mg/kg, i.p.) on protein expression of GLT-1 in P rats exposed to chronic ethanol drinking paradigm. One-way ANOVA revealed no significant difference in GLT-1 expression in the NAc-core, among all tested groups (F_{3, 28}=0.734, p>0.05, n=8/group), as shown in FIG. 2A. However, there was a significant difference in GLT-1 expression among the four groups in the NAc-shell (F₃, 28=4.85, p<0.01, n=8/group). Newman-Keuls multiple comparisons post-hoc analysis revealed a significant decrease in GLT-1 protein expression in the NAc-shell of the Ethanol group compared to control group (p<0.05), while its expression was significantly increased in groups treated with MC-100095 (50 mg/kg, i.p.) (p<0.05) or Ceftriaxone (200 mg/kg, i.p.) (p<0.01) in the NAc-shell as compared to Ethanol group. No significant changes were detected between control, Ethanol-MC (50 mg/kg), and Ethanol-CEF (200 mg/kg) groups as shown in FIG. 2B.

(5) Effect of MC-100093 (50 mg/kg, i.p.) and Ceftriaxone (200 mg/kg, i.p.) on the Expression of xCT in the NAc-Core and NAc-Shell:

FIGS. 2C-2D shows the effects of MC-100093 (50 mg/kg, i.p.) or ceftriaxone (200 mg/kg, i.p.) treatment on (C) protein expression of xCT in the NAc-core and (D) protein expression of xCT in the NAc-shell. In FIG. 2A, the upper panel shows representative immunoblot of xCT and β-tubulin in the NAc-core. In the lower panel, quantitative analysis revealed a non-significant difference in the expression of xCT among Control, Ethanol, Ethanol-MC (50 mg/kg), and Ethanol-CEF (200 mg/kg) in the NAc-core. In FIG. 2D, the upper panel shows representative immunoblot of xCT and β-tubulin in the NAc-shell, and in the lower panel, quantitative analysis revealed a significant downregulation in the expression of xCT in the Ethanol group compared to the control group. However, Ethanol-MC (50 mg/kg), and Ethanol-CEF (200 mg/kg) groups had significantly higher level of xCT compared to the ethanol group, with no significant change between control, Ethanol-MC (50 mg/kg), and Ethanol-CEF (200 mg/kg) groups, in the NAc-shell. Control group data were represented as 100% (relative to water-control). The values are expressed as mean±SEM (n=8/group), (*p<0.05 and **p<0.01).

One-way ANOVA revealed no significant difference in xCT protein expression levels in the NAc-core, among all tested groups (F_{3, 28}=0.265, p>0.05, n=8/group), as shown in FIG. 2C. However, there was a significant difference in protein expression of xCT among the four groups in the NAc-shell (F_{3, 28}=4.48, p<0.05, n=8-9/group). Newman-Keuls multiple comparisons post-hoc analysis showed a significant decrease in the xCT expression in the NAc-shell of the Ethanol group compared to control group (p<0.05). The analysis also showed that MC-100095 at 50 mg/kg (p<0.01) or Ceftriaxone at 200 mg/kg (p<0.05) significantly increased xCT expression in the NAc-shell as compared to the ethanol group. No significant changes were detected between control, Ethanol-MC (50 mg/kg), and Ethanol-CEF (200 mg/kg) groups as shown in FIG. 2D.

(6) Effect of MC-100093 (50 mg/kg, i.p.) and Ceftriaxone (200 mg/kg, Ip.) on the Expression of Metabotropic Glutamate Receptors in NAc-Core and NAc-Shell:

We next investigated the effects of MC-100093 (50 mg/kg, i.p.) or Ceftriaxone (200 mg/kg, i.p.) on protein expression of mGluRs.

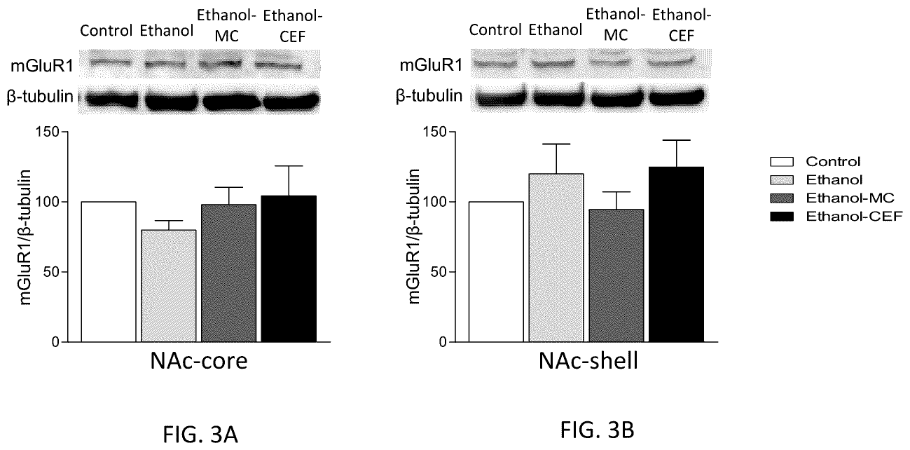

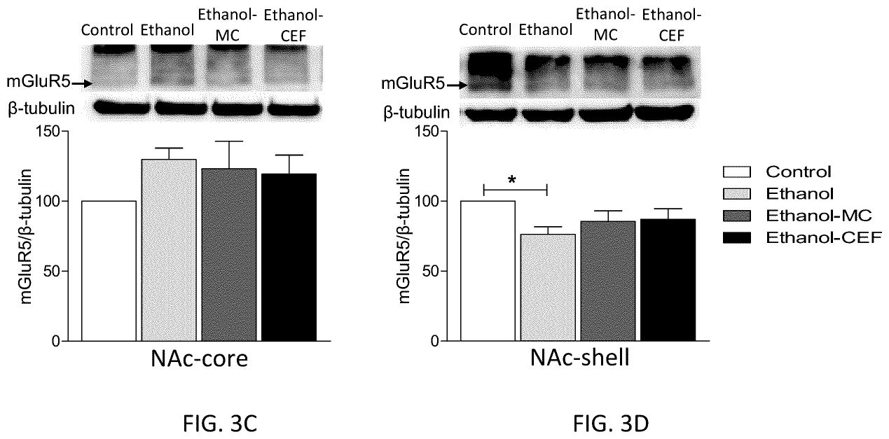

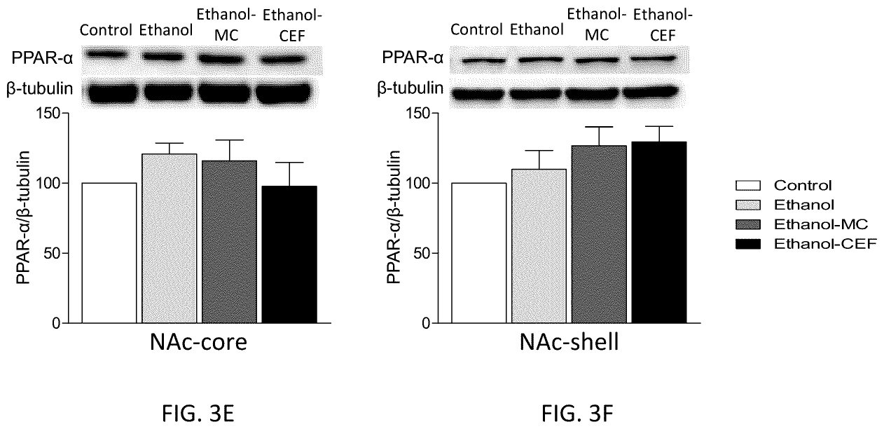

FIGS. 3A-3D show the effects of MC-100093 (50 mg/kg, i.p.) or ceftriaxone (200 mg/kg, i.p.) treatment on: (A) Protein expression of mGluR1 in the NAc-core, (B) Protein expression of mGluR1 in the NAc-shell, (C) Protein expression of mGluR5 in the NAc-core, and (D) Protein expression of mGluR5 in the NAc-shell. In FIG. 3A, the upper panel shows representative immunoblot of mGluR1 and β-tubulin in the NAc-core, and in the lower panel, quantitative analysis revealed a non-significant difference in the expression of mGluR1 among Control, Ethanol, Ethanol-MC (50 mg/kg), and Ethanol-CEF (200 mg/kg) in the NAc-core. In FIG. 3B, the upper panel shows representative immunoblot of mGluR1 and β-tubulin in the NAc-shell, and in the lower panel, quantitative analysis revealed non-significant differences in the expression of mGluR1 among Control, Ethanol, Ethanol-MC (50 mg/kg), and Ethanol-CEF (200 mg/kg) in the NAc-shell.

In FIG. 3C, the upper panel shows representative immunoblot of mGluR5 and β-tubulin in the NAc-core, and in the lower panel, quantitative analysis revealed a non-significant difference in the expression of mGluR5 among Control, Ethanol, Ethanol-MC (50 mg/kg), and Ethanol-CEF (200 mg/kg) in the NAc-core. In FIG. 3D, the upper panel shows representative immunoblot of mGluR5 and β-tubulin in the NAc-shell, and in the lower panel, Quantitative analysis revealed a significant downregulation in the expression of mGluR5 in Ethanol group compared to Control group in the NAc-shell. However, there was no significant change in the expression of mGluR5 in Ethanol-MC (50 mg/kg), and Ethanol-CEF (200 mg/kg) compared to either control or ethanol groups.

One-way ANOVA revealed no significant difference in mGluR1 expression level among all tested groups in the NAc-core (F_{3, 27}=0.802, p>0.05, n=7-8/group), and NAc-shell (F_{3, 28}=0.890, p>0.05, n=8/group), as shown in FIGS. 3A and 3B, respectively. Regarding mGluR5, there was no significant difference in protein expression among all groups in the NAc-core (F_{3, 27}=1.084, p>0.05, n=7-8/group), as shown in FIG. 3C. However, the protein expression of mGluR5 was significantly downregulated in ethanol group compared to control, Ethanol-MC (50 mg/kg), and Ethanol-CEF (200 mg/kg) groups in the NAc-shell (F₃, 28=2.707, p<0.05, n=8/group). No significant changes were detected between control, Ethanol-MC (50 mg/kg), and Ethanol-CEF (200 mg/kg) groups, as shown in FIG. 3D.

(7) Effect of MC-100093 (50 mg/kg, i.p.) and Ceftriaxone (200 mg/kg, i.p.) on the Expression of PPAR-α in the NAc-Core and NAc-Shell:

In FIG. 3E, the upper panel shows representative immunoblot of PPAR-α and β-tubulin in the NAc-core, and in the lower panel, quantitative analysis revealed non-significant differences in the expression of PPAR-α among Control, Ethanol, Ethanol-MC (50 mg/kg), and Ethanol-CEF (200 mg/kg) in the NAc-core. In FIG. 3F, the upper panel shows representative immunoblot of PPAR-α and β-tubulin in the NAc-shell, and in the lower panel, quantitative analysis revealed non-significant differences in the expression of PPAR-α among Control, Ethanol, Ethanol-MC (50 mg/kg), and Ethanol-CEF (200 mg/kg) in the NAc-shell. Control group data were represented as 100% (relative to water-control). The values are expressed as mean±SEM (n=7-8/group), (*p<0.05 and **p<0.01).

We did not detect significant changes in the protein expression of PPAR-α between all tested groups in NAc-core (F_{3, 28}=0.922, p>0.05, n=8/group) and NAc-shell (F₃, 20=1.609, p>0.05, n=6/group) as shown in FIGS. 3E and 3F, respectively.

### (8) Effect of Chronic Ethanol Exposure, MC-100093, and Ceftriaxone on Fat Deposition in the Liver:

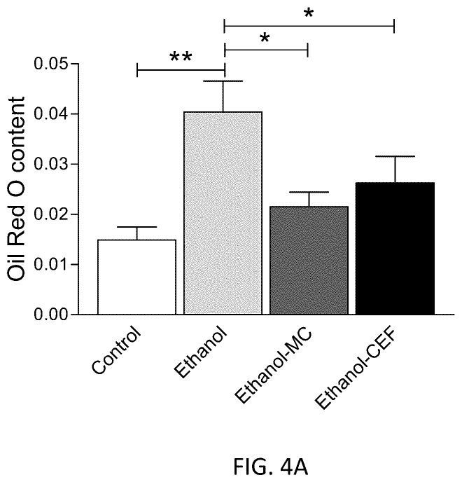

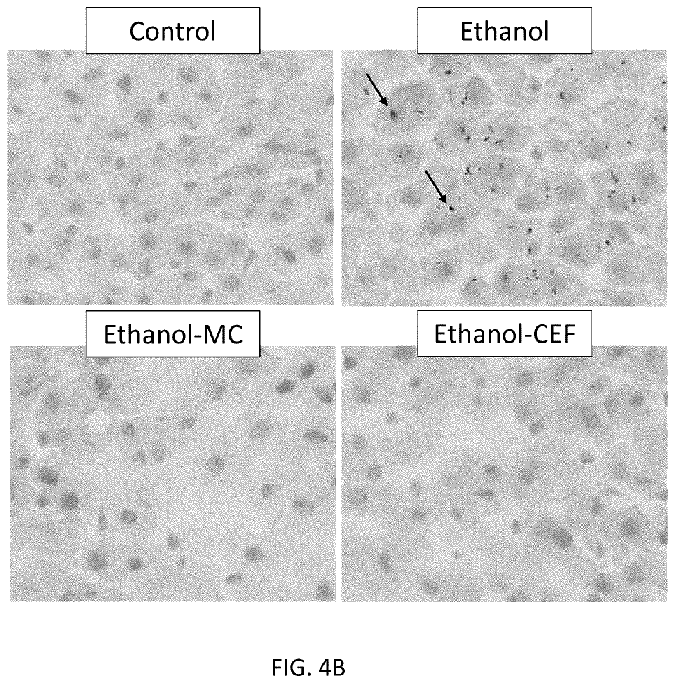

FIGS. 4A-4B show fat droplets in Oil Red O stained liver sections. As shown in FIG. 4A, quantitative analysis revealed a significant increase in liver fat content (high fat deposition) in ethanol group compared to control group. There was no significant difference in fat content between control, ethanol-MC, and ethanol-CEF groups. FIG. 4B shows representative Oil Red O stained liver sections of control, ethanol, ethanol-MC and ethanol-CEF rats. Higher fat content was observed in liver sections of the ethanol group as compared to the control, ethanol-MC or ethanol-CEF group. The values are expressed as mean±SEM (n=4-5/group), (*p<0.05).

In order to measure the fat deposition in rat liver tissues, Oil Red O staining used to calculate the liver fat content for each group. One-way ANOVA revealed a significant change in fat content between groups (F_{3, 15}=5.543, p<0.01, n=4-5/group. Newman-Keuls multiple comparisons post-hoc analysis revealed significantly higher fat droplet content in ethanol group (p<0.05) compared to control, ethanol-MC or ethanol-CEF groups. However, no significant changes observed between control, ethanol-MC or ethanol-CEF groups (FIG. 4A).

(9) Effects of MC-100093 (50 mg/kg, i.p.) and Ceftriaxone (200 mg/kg, i.p.) on the Protein Expression of PPARs in the Liver:

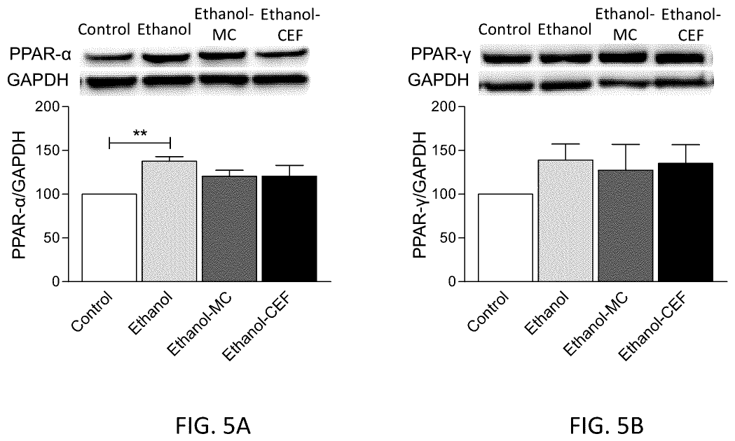

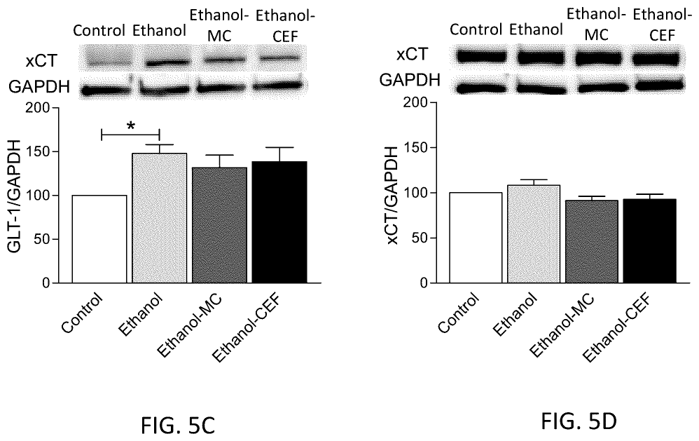

We also investigated the effects of MC-100093 (50 mg/kg, i.p.) or Ceftriaxone (200 mg/kg, i.p.) on protein expression of PPAR-α in the liver of p rats exposed to chronic ethanol drinking paradigm. FIGS. 5A-5B show Effects of MC-100093 (50 mg/kg, i.p.) or ceftriaxone (200 mg/kg, i.p.) on: (A) Protein expression of PPAR-α in the liver, and (B) Protein expression of PPAR-γ in the liver. In FIG. 5A, the upper panel shows representative immunoblot of PPAR-α and GAPDH in the liver, and in the lower panel: Quantitative analysis revealed a significant increase in the expression of PPAR-α in the ethanol group compared to the control group in the liver. One-way ANOVA revealed a significant change of PPAR-α protein expression (F_{3, 28}=4.223, p<0.05, n=8/group). Newman-Keuls multiple comparisons post-hoc analysis showed that PPAR-α protein expression was significantly upregulated in ethanol group compared to control group (p<0.01) and that treatment with MC-100093 (50 mg/kg, i.p.) or Ceftriaxone (200 mg/kg, i.p.) normalized PPAR-α level.

However, there was no significant change in the expression of PPAR-α in Ethanol-MC (50 mg/kg), and Ethanol-CEF (200 mg/kg) compared to either control or ethanol groups. As show in FIG. 5B, the protein expression of PPAR-γ was not changed significantly between all groups (F_{3, 28}=0.747, p>0.05, n=8/group). The upper panel shows representative immunoblot of PPAR-γ and GAPDH in the liver.

(10) Effects of MC-100093 (50 mg/kg, i.p.) and Ceftriaxone (200 mg/kg, i.p.) on the Protein Expression of GLT-1 and xCT in the Liver:

FIG. 5C shows the effect on protein expression of GLT-1 in the liver. The upper panel shows representative immunoblot of GLT-1 and GAPDH in the liver. In the lower panel, quantitative analysis revealed a significant increase in the expression of GLT-1 in ethanol group compared to control group in the liver. There was a significant change in GLT-1 protein expression as revealed by the statistical analysis (F_{3, 28}=3.042, p<0.05, n=8/group). Newman-Keuls multiple comparisons post-hoc analysis showed that GLT-1 protein expression was significantly upregulated in ethanol group compares to control group (p<0.05) and that treatment with MC-100093 (50 mg/kg, i.p.) or Ceftriaxone (200 mg/kg, i.p.) normalized GLT-1 level.

FIG. 5D shows the effect on protein expression of xCT in the liver. The upper panel shows representative immunoblot of xCT and GAPDH in the liver. In the lower panel, quantitative analysis revealed no significant differences in the expression of xCT between all groups. Control group data were represented as 100% (relative to water-control). The values are expressed as mean±SEM (n=8/group), (*p<0.05 and **p<0.01). One-way ANOVA revealed no significant change in xCT protein expression between all groups (F_{3, 28}=2.613, p>0.05, n=8/group.

Further studies were conducted to investigate the impact of MC-100093 at higher dose (100 mg/kg, i.p.) on ethanol consumption and glutamate transporter 1 (GLT-1) expression in medial prefrontal cortex (mPFC) subregions such as prelimbic mPFC (PL) and infralimbic mPFC (IL). Male P rats (n=16) were divided in three groups: a) Control water group was exposed to water only for 5 weeks and received i.p. saline on Week 6 for 5 days; b) Saline group was exposed to free choice of ethanol (15% and 30% and water) for 5 weeks, and received saline (i.p.) on Week 6 for 5 days; and c) MC-100093 group was exposed to free choice of ethanol for 5 weeks and received MC-100093 (100 mg/kg/day, i.p.) for 5 days on Week 6. The results showed that MC-100093 attenuated ethanol intake and decreased ethanol preference. Furthermore, MC-100093 increased GLT-1 expression in both subregions of mPFC such as PL and IL.

### (11) Effects of MC-100093 on Ethanol Consumption and Water Consumption:

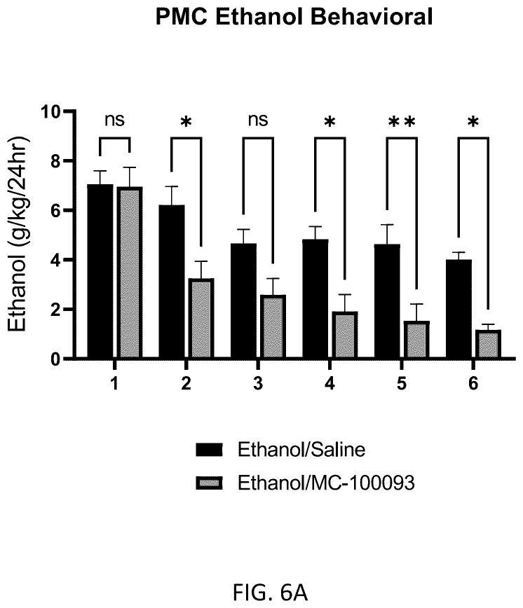

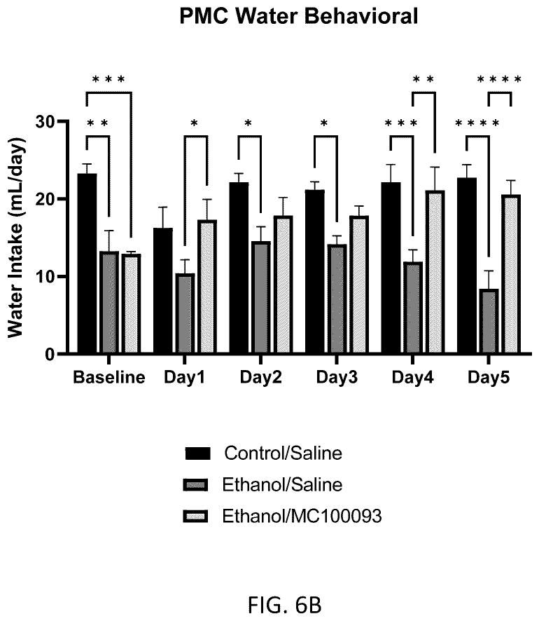

FIGS. 6A-6B show the effects of five consecutive days of MC-100093 (100 mg/kg, i.p.) or saline (i.p.) treatment on (A) ethanol consumption (g/kg/24 hr), and (B) water intake (mL/day). Statistical analyses revealed that treatment with MC-100093 significantly reduced ethanol consumption on Day 1, 3, 4, and 5 in ethanol/MC-100093, associated with significant increase in water intake compared to ethanol/saline group. The values are expressed as mean±SEM (n=4-5/group), (*p<0.05 and **p<0.01, ***p<0.001, and ****p<0.0001).

MC-100093 (100 mg/kg, i.p.) for five days reduced ethanol intake on Days 2, 4, 5, 6 on Week 6 after rats were exposed to five weeks of free choice ethanol (15% and 30%) and water (FIG. 6A). In addition, increase in water consumption was observed on Days 1-5 of Weeks 6 in rats treated with MC-100093 as compared to rats treated with saline vehicle (FIG. 6B).

(12) Effects of MC-100093 (100 mg/kg, i.p.) on Body Weight:

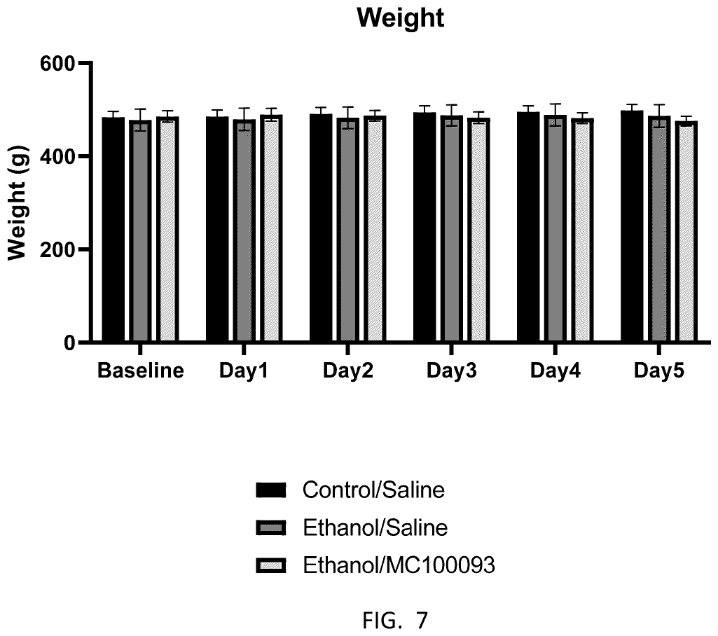

Referring to FIG. 7, there was no significant effects of MC-100093 on body weight among all groups. (n=4-5/group). Statistical analysis didn't show any difference in body weight of rats exposed to water, ethanol, or ethanol and MC-100093 (FIG. 2). Mc-100093 didn't change the body weight of rats.

(13) Effects of MC-100093 (100 mg/kg, i.p.) on GLT-1 Expression in Subregions of Medial Prefrontal Cortex:

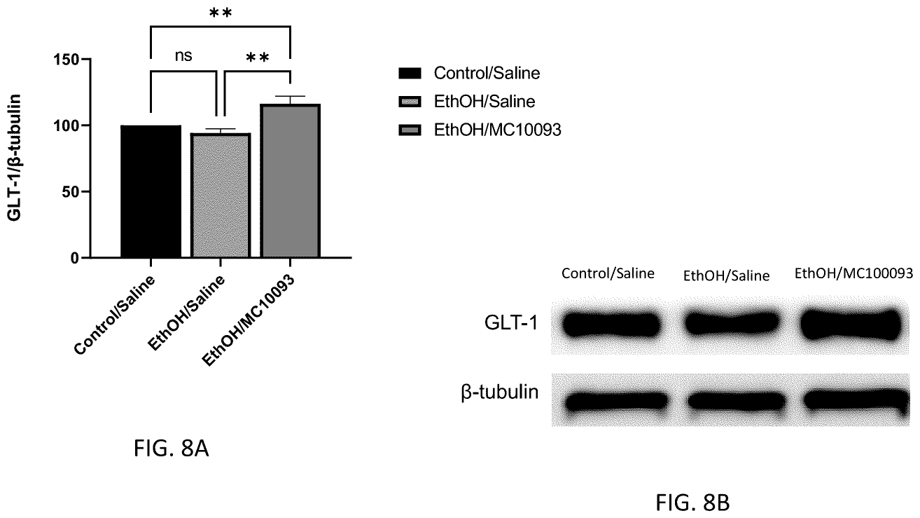

FIGS. 8A-8B show the effects of five consecutive days of MC-100093 (100 mg/kg, i.p.) on GLT-1 expression in the infralimbic prefrontal cortex (IL). Quantitative analysis showed that Ethanol/MC-100093 group was associated with a significantly higher levels of GLT-1 expression compared to the ethanol/saline. The values are expressed as mean±SEM (n=5/group), (*p<0.05 and **p<0.01).

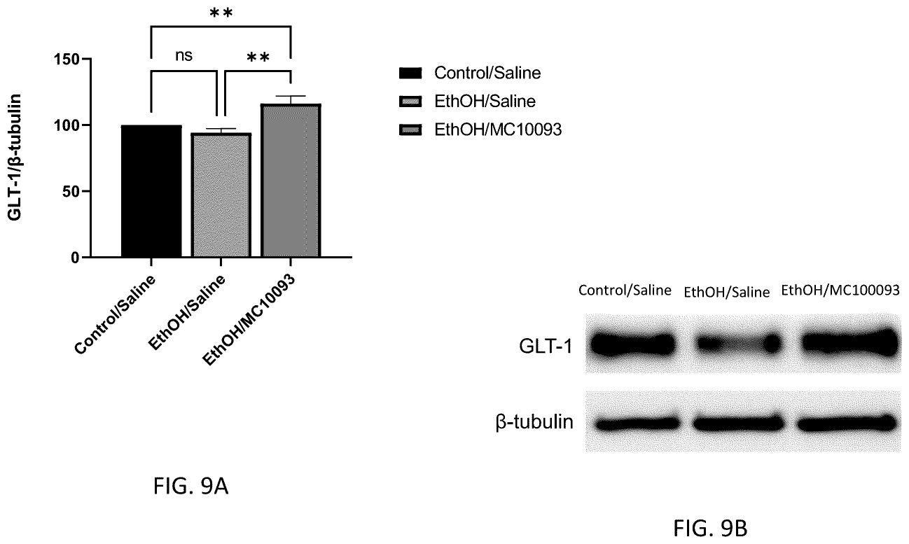

FIGS. 9A-9B show the effects of five consecutive days of MC-100093 (100 mg/kg, i.p.) on GLT-1 expression in the prelimbic frontal cortex (PL). Quantitative analysis showed no significant changes in GLT-1 level between control/saline, Ethanol/saline, and Ethanol/MC-100093 groups. However, MC-100093 were associated with a non-statistically significant increase of GLT-1 expression. The values are expressed as mean±SEM (n=5/group), (**p<0.01).

So we found that treatment with MC-100093 (100 mg/kg, i.p.) for five days increased the expression of GLT-1 in infralimbic prefrontal cortex (IL) of ethanol/MC-100093 group as compared to control/saline and ethanol/saline groups (FIGS. 8A-8B). Similarly, we found that MC-100093 upregulated the expression of GLT-1 in the prelimbic prefrontal cortex of ethanol/MC-100093 group as compared to control/saline and ethanol/saline groups (FIGS. 9A-9B). This clearly indicates that MC-100093 treatment upregulates GLT-1 to prevent any dysfunction in glutamate transport that may occur with chronic consumption of free choice of ethanol.

### A-3. Discussion

In the current study, we showed that chronic ethanol consumption was associated with dysregulation of the glutamatergic systems in the brain and liver of P rats. More specifically, the expression of GLT-1, xCT, and mGluR5 were downregulated in the NAc-shell while the liver exhibited upregulation in GLT-1 protein expression. Moreover, the steatotic liver in alcohol dependent rats showed higher protein expression of PPAR-α. More importantly, the novel beta lactam compound, MC-100093, attenuated ethanol drinking behavior and normalized changes in several protein targets associated with chronic ethanol drinking. It is noteworthy that MC-100093 is orally bioavailable with no antibiotic activity, and a potent GLT-1 upregulator.

Our results demonstrate that MC-100093 may be a potential treatment for alcohol dependence with preferable characteristics (i.e. high oral bioavailability and no antibiotic actions) compared to ceftriaxone. Nevertheless, ceftriaxone (200 mg/Kg, i.p.) was able to attenuate drinking behavior to a greater extent than MC-100093 (50 mg/Kg). This is probably due to the use of one low dose of MC-100093 (50 mg/kg, i.p.), which represents one of the limitations in this study.

However, we have further tested higher dose of MC-100093 (100 mg/kg, i.p.) and showed about 50% reduction in ethanol consumption as compared to control vehicle group. MC-100093 (100 mg/kg, i.p.) showed effect similar to that of ceftriaxone (200 mg/Kg, i.p.). It is important to note that MC-100093 (50 mg/kg, i.p.) attenuation of ethanol drinking was associated with normalized GLT-1 and xCT levels in the NAc-shell. This is in line with our finding that demonstrated chronic ethanol drinking induced downregulation of glutamate transporters in the NAc-shell and treatment with GLT-1 upregulators attenuated ethanol drinking behaviors.

We have also found that MC-100093 at higher dose (100 mg/kg, i.p.) upregulated GLT-1 expression in the subregions of medial prefrontal cortex such as PL and IL. Furthermore, we found that chronic exposure to ethanol induced glucocorticoid resistance and neuroinflammatory response in the NAc-shell but not in the core, pointing to the involvement of the NAc-shell in the pathology of ethanol dependence. Likewise, chronic ethanol consumption reduced the expression of mGluR5 in the NAc-shell, and its normalization was associated with attenuation of drinking behavior and neuroinflammation.

It is noteworthy that mGluR5 signaling is strongly implicated in the development and maintenance of ethanol dependence in animal models. For instance, blockade of mGluR5 action resulted in reduction in ethanol consumption by mice and reduction in both consumption and ethanol self-administration under a progressive ratio (PR) schedule of reinforcement by P rats. Moreover, activation of mGluR5 resulted in anti-inflammatory effects by inhibiting microglial activation and the release of inflammatory mediators.

In this study, we further confirmed the role of mGluR5 in ethanol drinking behavior by demonstrating that chronic ethanol induced downregulation of mGluR5 in the NAc-shell. Additionally, MC-100093 or ceftriaxone treatment was associated with normalizing levels of mGluR5 in alcohol dependent animals, which may contribute to the attenuation of ethanol drinking behavior. Together, the previous studies along with the current findings suggest that the NAc-shell is strongly involved in ethanol dependence. In this regard, chronic ethanol exposure induces dysregulation of the glutamatergic systems, and modulation of glutamate receptors/transporters in the NAc-shell offers treatment method for treating of ethanol dependence

The role of PPARs in ethanol dependence is well documented. Body of research has shown that PPAR agonists reduce ethanol drinking behavior in animal models. For instance: Gemfibrozil, a PPAR-α agonist, reduced ethanol drinking behavior in rats; pioglitazone and rosiglitazone reduced ethanol drinking and stress-induced relapse in rats model. One hypothesis for the mechanisms underlying PPAR-associated reductions in ethanol consumption is through anti-inflammatory actions in the brain that modulate the neuroimmune system. However, PPAR-α activation in the liver stimulates hepatic catalase and hydrogen peroxide that leads to ethanol aversion via conversion of ethanol into acetaldehyde, which represents another possible mechanism.

To assess this hypothesis, we used our chronic ethanol drinking model to measure the expression of PPARs in the liver and brain. The expression of PPAR-α in the NAc-core and NAc-shell were not significantly changed [we couldn't detect the PPAR-γ in the brain in our experimental conditions, probably due to lower expression compared to other organs. In the liver, although the PPAR-γ expression was not changed, we found that chronic ethanol exposure significantly increased PPAR-α level. We found that our ethanol drinking paradigm associated with the development of fatty liver. Exposure to ethanol for four weeks did not alter the expression of PPAR-α but was associated with reduced PPAR/RXR binding to its consensus sequence in mice model. Our results suggest that increased PPAR-α expression in P rats exposed to ethanol for six weeks increases liver fat contents. Of note, it is suggested that PPAR-α agonists reduce ethanol consumption through activation of PPAR-α in the liver. Interestingly, our results showed that the expression of GLT-1 was upregulated in the liver, with no change in xCT levels. Studies have shown that PPAR-γ activation increased the expression of GLT-1 at the transcriptional level. However, little is known about the involvement of PPAR-α in the expression of GLT-1. Nevertheless, it was shown that PPAR-α signaling promotes GLT-1 endocytosis in astrocytes.

Our findings in this study may suggest connection between PPAR-α and GLT-1 activities. Further, we showed that treatment with MC-100093 or ceftriaxone alleviated the liver steatosis and normalize the PPAR-α and GLT-1 levels in the liver. Thus, this study revealed the beneficial effects of both MC-100093 and ceftriaxone in ethanol dependence and its consequences in the brain and liver.

In summary, we showed here that chronic ethanol drinking induced dysregulation in glutamatergic systems in the brain and liver. In addition to the fatty liver changes, chronic ethanol consumption was associated with increased expression of PPAR-α in the liver. Treatment with MC-100093 or ceftriaxone attenuated ethanol drinking behavior, which was associated with normalized changes in target proteins. This study presents a new potential candidate, MC-100093, for the treatment of ethanol dependence.

### B. Effects of Compound on Ethanol Drinking and the Expression of Astrocytic Glutamate Transporters

We have studied the effects of the exemplary compound, MC-100093, on ethanol drinking and the expression of astrocytic glutamate transporters in the mesocorticolimbic brain regions of male and female alcohol-preferring rats.

Chronic ethanol consumption increased extracellular glutamate concentrations in several reward brain regions. Glutamate homeostasis is regulated in majority by astrocytic glutamate transporter 1 (GLT-1) as well as the interactive role of cystine/glutamate antiporter (xCT). In our study, we aimed to determine the attenuating effects of a novel beta-lactam compound, MC-100093, lacking the antibacterial properties, on ethanol consumption and GLT-1 and xCT expression in the subregions of nucleus accumbens (NAc core and NAc shell) and medial prefrontal cortex (Infralimbic, mPFC-IL and Prelimbic, mPFC-PL) in male and female alcohol-preferring (P) rats.

Female and male rats were exposed to free access to ethanol (15% v/v) and (30% v/v) for five weeks, and on Week 6, rats were administered 100 mg/kg (i.p) of MC-100093 or saline for five days. MC-100093 reduced ethanol consumption in both male and female P rats from Day 1-5. Additionally, MC-100093 upregulated GLT-1 and xCT expression in mPFC and NAc subregions as compared to ethanol-saline groups in female and male rats. Chronic ethanol intake reduced GLT-1 and xCT expression in the IL and PL in female and male rats, except there was no reduction in GLT-1 expression in the mPFC-PL in female rats. Although, MC-100093 upregulated GLT-1 and xCT expression in the subregions of NAc, we did not observe any reduction in GLT-1 and xCT expression with chronic ethanol intake in female rats. These findings strongly suggest that MC-100093 treatment effectively reduced ethanol intake and upregulated GLT-1 and xCT expression in the mPFC and NAc subregions in male and female P rats.

Among many projecting glutamatergic pathways, there are glutamatergic signals that project from the medial prefrontal cortex (mPFC), amygdala, and hippocampus to the nucleus accumbens (NAc). NAc is divided into two distinct sub-regions termed the nucleus accumbens shell (NAc-shell) and the nucleus accumbens core (NAc-core).

Glutamatergic projections from the mPFC to the NAc are suggested to be involved in regulating reward-related behaviors. The mPFC regulates reward and motivation, and its disruption triggers the loss of control over certain compulsive drug-seeking behaviors. There are at least four subregions in the mPFC, two of which, mPFC-infralimbic (mPFC-IL) and mPFC-prelimbic (mPFC-PL), have been shown to regulate the behavior differently.

It is important to note that disruption of mPFC-NAc glutamatergic pathways might contribute to the development of dependence to ethanol. Glutamate homeostasis is disrupted with ethanol exposure and dependence, leading to increased extracellular glutamate concentrations in mesocorticolimbic brain regions.

Several glutamate transporters mediate the regulation of extracellular glutamate concentration. Among these glutamate transporters, glutamate transporter 1 (GLT-1, its human homolog is excitatory amino acid transporter 2, EAAT2) regulates the uptake of the majority of extracellular glutamate concentrations in the brain. Extensive studies from our laboratory demonstrated clearly that chronic ethanol consumption downregulated the expression of GLT-1 in mesocorticolimbic brain regions, and this effect was associated with increased extracellular glutamate concentration in the brain of alcohol-preferring (P) rats. Alternatively, cystine-glutamate exchanger (xCT) is another glutamate transporter that is responsible for the release of astrocytic glutamate in exchange for cystine. Several studies from our lab showed that chronic ethanol exposure decreased xCT expression in several brain reward regions of P rats.

Our lab has been studying the effects of GLT-1 upregulators on attenuating the effects of chronic exposure to ethanol. Thus, our studies have clearly demonstrated that ceftriaxone, a beta-lactam antibiotic known to upregulate GLT-1, decreased ethanol consumption in rats, and this effect was associated with attenuation of downregulation of GLT-1 as well as xCT expression and consequently normalization of the extracellular glutamate concentration in the brain reward regions such as the NAc. In addition to ceftriaxone, other beta-lactam antibiotics are effective on attenuating ethanol consumption and relapse behaviors, in part through upregulation of GLT-1 and xCT expression in brain reward regions such as NAc and mPFC.

Importantly, our study revealed the efficacy of a novel beta-lactam, MC-100093, in reducing ethanol intake in P rats, and this effect was associated with upregulation of GLT-1 in NAc. It is important to note that the pharmacokinetics of MC-100093 were performed in a study, which showed that MC-100093 displayed 28% oral bioavailability (F=28%) in rats, and the drug was associated with a 14% brain/plasma ratio after intraperitoneal (i.p) injection. This latter study revealed that MC-100093 was associated with a 23.5% enhancement of glutamate uptake with an IC50 of 0.1 μM in an astrocyte-neuron co-culture model. In addition, MC-100093 lacks antimicrobial activity against gram-positive and gram-negative bacteria.

In this study, we examined the effects of MC-100093 on attenuating ethanol consumption in both male and female P rats. Additionally, this study explored the neurocircuits involving the mPFC subregions (mPFC-IL and mPFC-PL) and the NAc subregions (NAc-shell and NAc-core) to determine any changes in the expression of GLT-1 and xCT in P rats of both sexes.

### B-1. Materials and Methods

### Animals

Male and female alcohol-preferring (P) rats were acquired from Indiana University School of Medicine, Indianapolis, IN at the age of 80-85 days. All animals were single housed in a room with temperature at 22° C. and 50% humidity in a 12 h light/dark cycle. Rats had free access to food and water throughout the experimental procedures. All the experimental procedures were approved by the University of Toledo Institutional Animal Care and Use Committee (IACUC) under IACUC protocol #400160.

### Ethanol Intake Measurements

At the age of 90-100 days, 5-7 male and female rats were assigned randomly to three groups: (1) Water-drinking (Water-Saline) group with no access to ethanol and served as a control group; (2) Ethanol-Saline group, which was exposed to continuous free-choice access to ethanol (15% and 30%, v/v, concurrently), and water for five weeks, and saline i.p. injections were performed from Days 1-5 on Week 6; and (3) Ethanol-MC-100093 group, which had continuous free-choice access to ethanol (15% and 30%, v/v), and water for five weeks, and MC-100093 i.p. injections were performed from Days 1-5 on Week 6. Ethanol and water intake were measured as g/kg/day. Rats were required to meet the criterion of an average ethanol consumption of 4 g/kg/day to be included in the study, for at least 2 weeks before saline vehicle or MC-100093 i.p. injections, as adopted in previous studies. Average ethanol and water consumptions throughout Week 5 served as a baseline. At Week 6 of the experiment, ethanol-MC-100093 group received MC-100093 at a dose of 100 mg/kg (i.p.) once daily for five days, and ethanol-saline and water-saline groups received saline vehicle as an i.p injection for five consecutive days. Water and ethanol were measured daily 24 hours after the first i.p. injections of saline or MC-100093 and 24 hours after the last i.p. injections of saline or MC-100093.

### Brain Tissue Harvesting

After 24 hours of receiving the last injection, rats were euthanized by CO₂ inhalation followed by decapitation with a guillotine. Brains were harvested and immediately frozen on dry ice and stored at −80° C. Cryostat (Leica) was used to isolate the mPFC subregions (mPFC-IL and mPFC-PL), and subregions of the NAc (NAc-core and NAc-shell). Brains were micro-punctured using the stereotaxic coordinates following the Rat Brain Atlas. The isolated brain regions were stored at −80° C. for subsequent protein detection using Western Blot assay.

### Western Blot Analyses

Immunoblot assays were performed to determine the expression of GLT-1, xCT and β-tubulin in mPFC-IL, mPFC-PL, NAc-core, and NAc-shell of all groups as described previously. Briefly, brain samples were homogenized in lysis buffer supplemented with protease inhibitor and total protein was quantified using protein assay (Bio-Rad, Hercules, CA, USA). Equal amount of the samples was loaded on polyacrylamide gel (10-20%).

Subsequently, proteins were transferred on a PVDF membrane and blocked with 5% milk in Tris-buffered saline Tween-20 (1×TBST) for 30 minutes at room temperature. Membranes were then incubated overnight at 4° C. with one of the following antibodies: rabbit anti-GLT-1 (1:1000, Abcam, AB41621), and/or rabbit anti-xCT (1:1000, Abcam, AB175186). Mouse anti-β-tubulin (1:1000; Cell Signaling D71G5) was used as a loading control antibody. On the following day, membranes were washed five times with 1×TBST followed by incubation with appropriate secondary antibody (1:4000) for 60 minutes at room temperature. Chemiluminescent reagents (Super Signal West Pico, Pierce Inc.) were used to detect proteins using a ChemiDoc imaging system (BioRad, USA).

GLT-1 and xCT expression were normalized against β-tubulin, a control loading protein. Imaging System and ImageJ software version 1.53a were used to quantify and analyze the expression of GLT-1 and xCT in the subregions of the mPFC and NAc. Data from water-control group were represented as 100% and all other values were expressed relative to this control to evaluate the changes in protein expression.

The ratios for treated animals in the ethanol/saline and ethanol/MC-100093 groups were normalized to the mean ratios for the control group (water/saline group). The control ratio was set at 100, and the results from each of the treated groups were expressed as a percentage relative to the water/saline group value of 100%. We normalized the ethanol/saline and ethanol/MC-100093 groups data to water/saline group to reduce any differences of contrast with Western blots for each set of groups (control/water/saline, ethanol/saline, and ethanol/MC-100093 groups).

### Statistical Analyses

All statistical analyses were conducted using GraphPad Prism (10). Two-way (mixed) ANOVA followed by Bonferroni multiple comparison post-hoc test was performed to analyze daily ethanol intake, average daily water intake and body weight. One-way ANOVA followed by Newman-Keuls post hoc tests were used to analyze Western blot data. All statistical analyses data were reported as a p<0.05 of significance.

### B-2. Results

### (1) Effect of MC-100093 Treatment on Ethanol Consumption, Ethanol Preference and Body Weight in Male P Rats

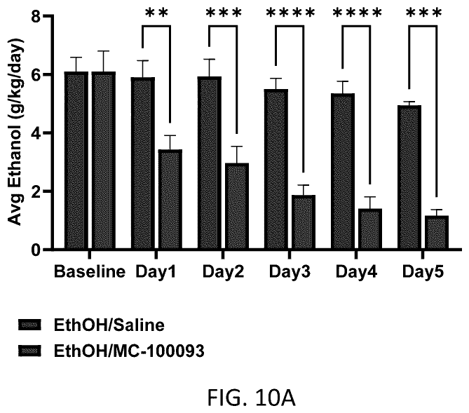

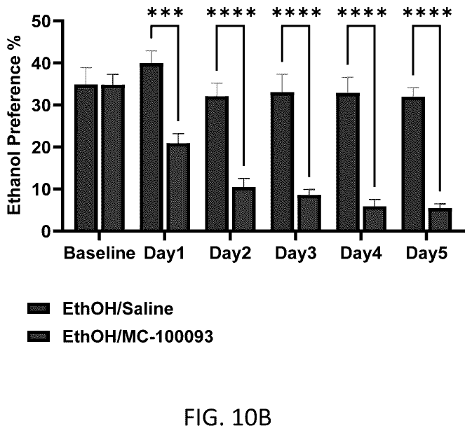

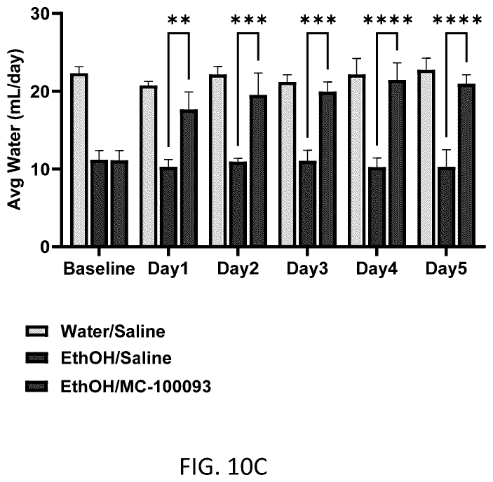

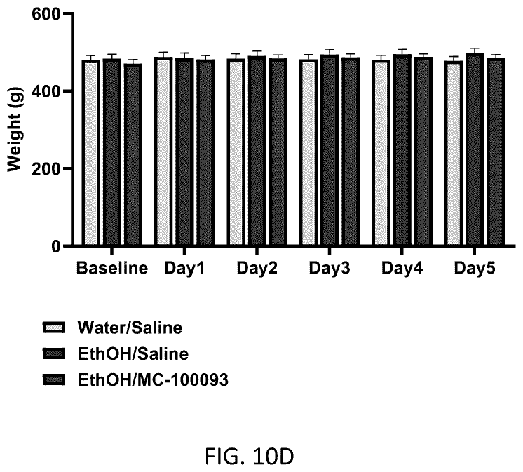

Statistical analysis using two-way ANOVA revealed significant main effects of Day (F_{5, 61}=10.04, P<0.0001), and Treatment (F_{1, 61}=88.97, P<0.0001) as well as a significant Treatment×Day interaction (F_{5, 61}=4.754, P=0.0010). Bonferroni multiple comparison test showed a significant decrease in ethanol consumption from Day 1 through Day 5 of treatment in the ethanol-MC-100093 group compared to the ethanol-saline group (FIG. 10A).

Ethanol preference was calculated as total ethanol consumption/total fluid consumption×100 from daily alcohol and water consumption. Two-way ANOVA revealed a significant main effect of Day (F_{5, 71}=9.958, p<0.0001) and Treatment (F1,71=122.8, p<0.0001) as well as a significant Treatment×Day interaction (F_{5, 71}=5.815, p=0.0001). Bonferroni multiple comparison test showed a significant decrease in percent ethanol preference in ethanol-MC-100093 group as compared to the ethanol-saline group starting on Day 1 through Day 5 (FIG. 10B).

Statistical analysis of water consumption data revealed a significant Treatment×Day interaction (F_{10, 9}5=2.219, p=0.0228) and Treatment (F_{2, 95=70.46}, p<0.0001). Bonferroni multiple comparison test showed a significant increase in water consumption from treatment Day 1 through Day 5 in the ethanol-MC-100093 group compared to the ethanol-saline group (FIG. 10C). Body weights (g) of rats were monitored throughout the study. MC-100093 treatment had no significant effect on body weights in male P rats (FIG. 10D).

### (2) Effect of MC-100093 Treatment on Ethanol Consumption, Ethanol Preference and Body Weight in Female P Rats

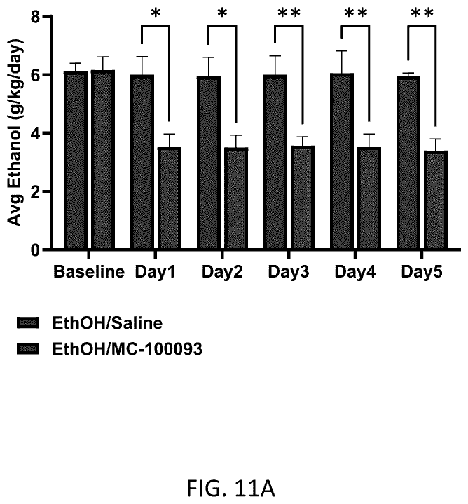

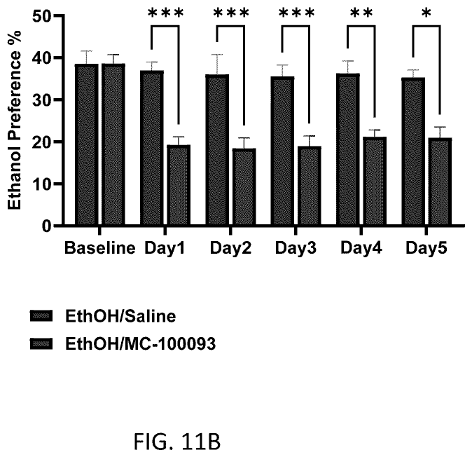

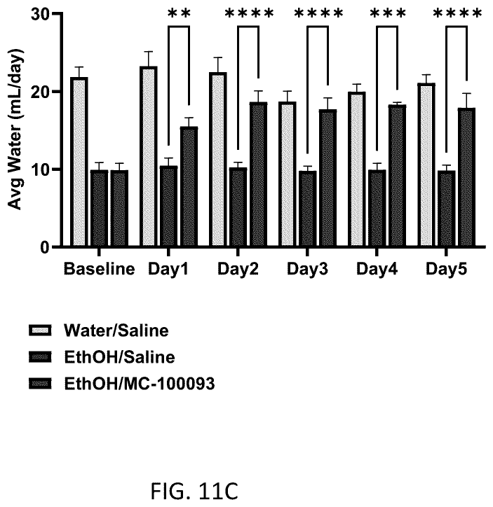

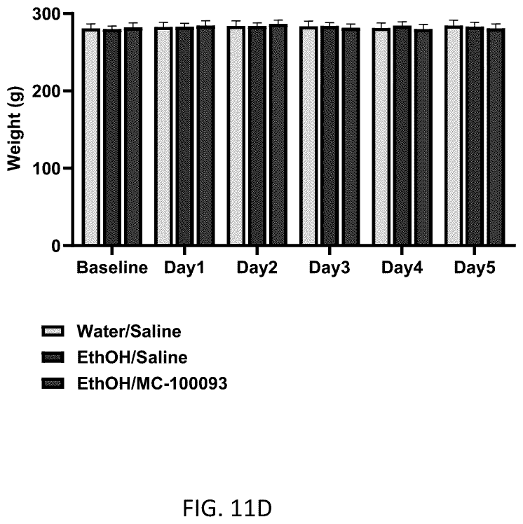

Two-way ANOVA revealed significant main effects of Day (F_{5, 54}=3.014, P=0.0180), and Treatment (F_{1, 54}=49.92, P<0.0001) as well as a significant Treatment×Day interaction (F_{5, 54}=2.477, P=0.0431). Bonferroni multiple comparison test showed a significant decrease in ethanol consumption from Day 1 through Day 5 in the ethanol-MC-100093 group as compared to the ethanol-saline group (FIG. 11A).

Two-way ANOVA revealed a significant main effect of Day (F_{5, 55}=5.999, p=0.0002) and Treatment (F1,55=67.73, p<0.0001) as well as a significant Treatment×Day interaction (F_{5, 55}=3.608, p=0.0068). Bonferroni multiple comparison test showed a significant decrease in percent ethanol preference in ethanol-MC-100093 group as compared to the ethanol-saline group starting on Day 1 through Day 5 (FIG. 11B).

Statistical analysis of water consumption data revealed a significant a significant main effect of Day (F_{5, 72}=2.725, p=0.0260) and Treatment (F_{2,72}=128.3, p<0.0001) and a significant Treatment×Day interaction (F_{10, 72}=3.709, p=0.0005). Bonferroni multiple comparison test showed a significant increase in water consumption from Day 1 through Day 5 in the ethanol-MC-100093 group as compared to the ethanol-ealine group (FIG. 11C). Body weights (g) of rats were monitored throughout the study. MC-100093 treatment had no significant effect on body weights in female P rats (FIG. 11D).

(3) Effect of MC-100093 on the Expression of GLT-1 in the mPFC-IL of Male and Female P Rats

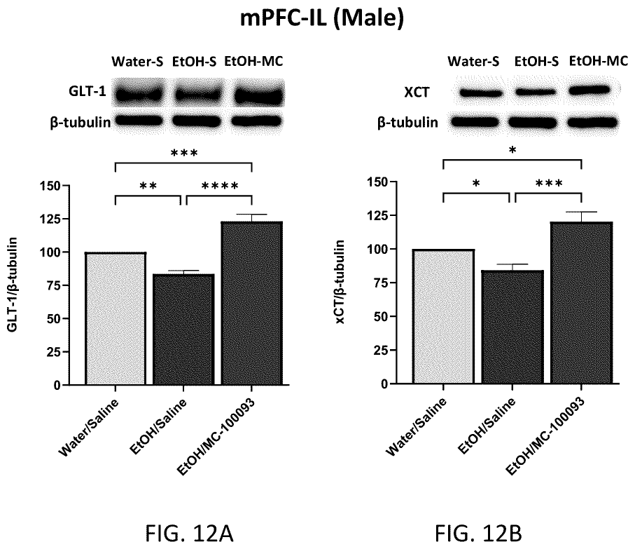

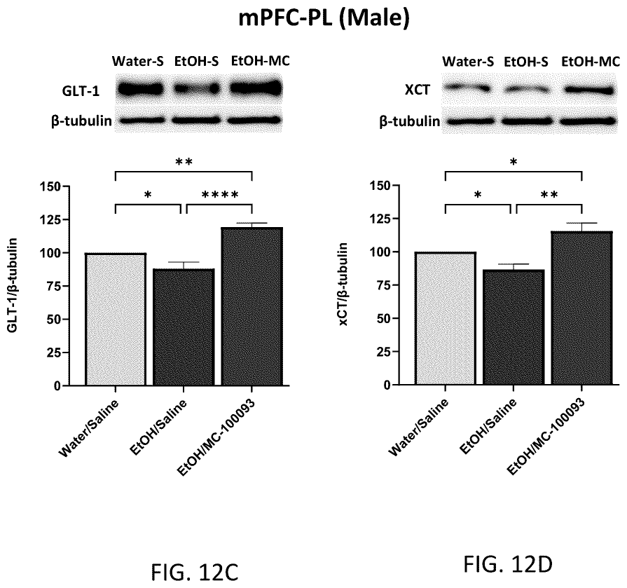

One-way ANOVA showed a significant difference in GLT-1 expression in the mPFC-IL (F_{2, 12}=34.30, p<0.0001) in male P rats (FIG. 12A). Newman-Keuls post-hoc analysis revealed a significant decrease in GLT-1 expression in the mPFC-IL of the ethanol-saline group as compared to the water-saline group (p<0.01), while MC-100093 attenuated ethanol-induced downregulation of GLT-1 expression (p<0.0001, p<0.001 respectively) (FIG. 12A).

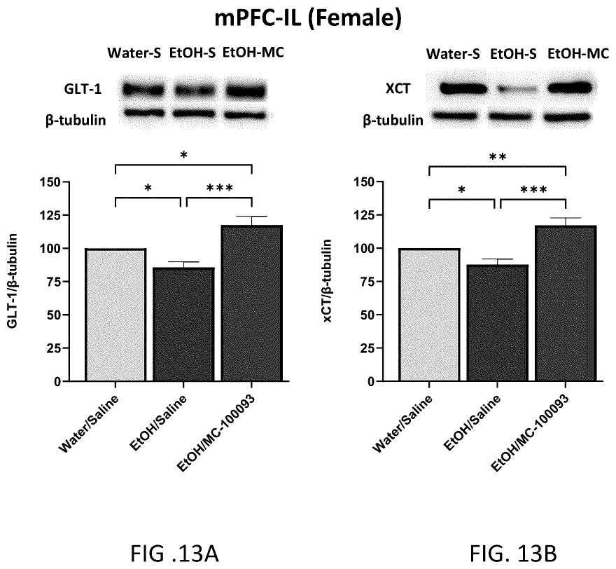

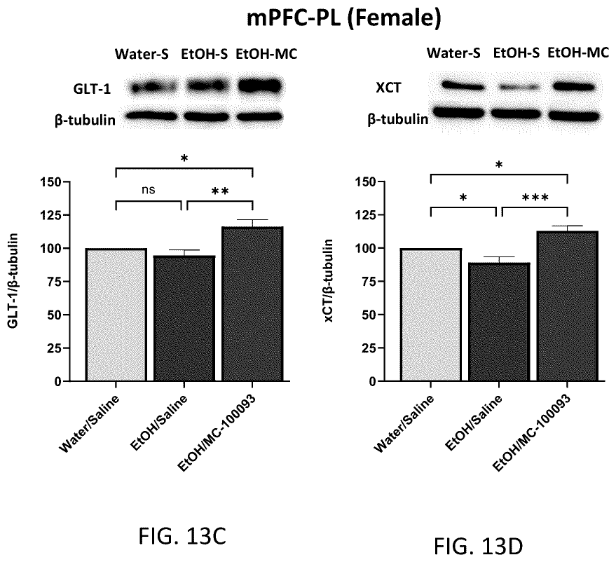

Alternatively, there was a significant difference in GLT-1 expression among the three groups in the mPFC-IL of female P rats (F_{2, 12}=12.47, p<0.0012) (FIG. 13A). Newman-Keuls post-hoc analysis showed MC-100093 upregulated GLT-1 expression in the mPFC-IL of female rats as compared to ethanol-saline group (p<0.001) and water-saline group (p<0.05), whereas a significant reduction in the expression of GLT-1 was found in the ethanol-saline group as compared to the water-saline group (p<0.05) (FIG. 13A).

(4) Effect of MC-100093 on the Expression of xCT in the mPFC-IL of Male and Female P Rats

One-way ANOVA revealed a significant difference in the expression of xCT in the mPFC-IL of male P rats among all tested groups (F_{2, 12}=13.81, p<0.0008) (n=5/group) (FIG. 12B). Newman-Keuls revealed a significant decrease in the expression of xCT in the mPFC-IL of the ethanol-saline group as compared to the water-saline group (p<0.05), while treatment with MC-100093 reversed the effect of ethanol and significantly increased the expression of xCT (p<0.001) (FIG. 12B).

In female P rats, there was a significant difference in xCT expression among the three groups in the mPFC-IL (F₂, 12=14.04, p<0.0007) (FIG. 13B). Newman-Keuls post-hoc analysis showed a significant upregulation in the expression of xCT in MC-100093-treated female rats as compared to ethanol-saline and water-saline groups (p<0.001 and p<0.01 respectively), whereas a significant reduction in the expression of xCT was found in the ethanol-saline group as compared to the water-saline group (p<0.05) (FIG. 13B).

(5) Effect of MC-100093 on the Expression of GLT-1 in the mPFC-PL of Male and Female P Rats

In male P rats, one-way ANOVA showed a significant difference in GLT-1 expression in the PFC-PL among all tested groups (F_{2, 12}=22.65, p<0.0001) (FIG. 12C). Newman-Keuls post-hoc analysis revealed a significant decrease in GLT-1 expression in the mPFC-PL of the ethanol-saline group as compared to the water-saline group (p<0.05), while its expression was significantly increased in group treated with MC-100093 (p<0.001) as compared to the ethanol-saline group and water-saline group (p<0.01) (FIG. 12C).

Furthermore, there was a significant difference in GLT-1 expression among the three groups in the mPFC-PL of female P rats (F_{2, 12}=8.886, p=0.0043) (FIG. 13C). Newman-Keuls post-hoc analysis showed a significant upregulation of GLT-1 expression in the mPFC-PL of MC-100093-treated group as compared to ethanol-saline and water-saline groups (p<0.01 and p<0.05 respectively) (FIG. 13C). No significant changes were detected between ethanol-saline and water-saline groups in the expression of GLT-1 in mPFC-PL of female P rats.

(6) Effect of MC-100093 on the Expression of xCT in the mPFC-PL of Male and Female P Rats

One-way ANOVA revealed a significant difference in the expression of xCT in the mPFC-PL of male P rats among all tested groups (F_{2, 12}=11.79, p=0.0015) (n=5/group) (FIG. 12D). Newman-Keuls test revealed a significant decrease in the expression of xCT in the mPFC-PL of the ethanol-saline group as compared to the water-saline group (p<0.05), while treatment with MC-100093 reversed the effect of ethanol and significantly increased the expression of xCT (p<0.01) (FIG. 12D).

In female P rats, there was a significant difference in xCT expression among the three groups in the mPFC-PL (F_{2, 11}=13.31, p=0.0009) (FIG. 13D). Newman-Keuls post-hoc analysis showed a significant upregulation of the expression of xCT in MC-100093-treated female rats as compared to ethanol-saline and water-saline groups (p<0.001 and p<0.05 respectively), whereas a significant reduction in the expression of xCT was found in the ethanol-saline group as compared to the water-saline group (p<0.05) (FIG. 13D).

### (7) Effect of MC-100093 on the Expression of GLT-1 in the NAc-Shell of Male and Female P Rats

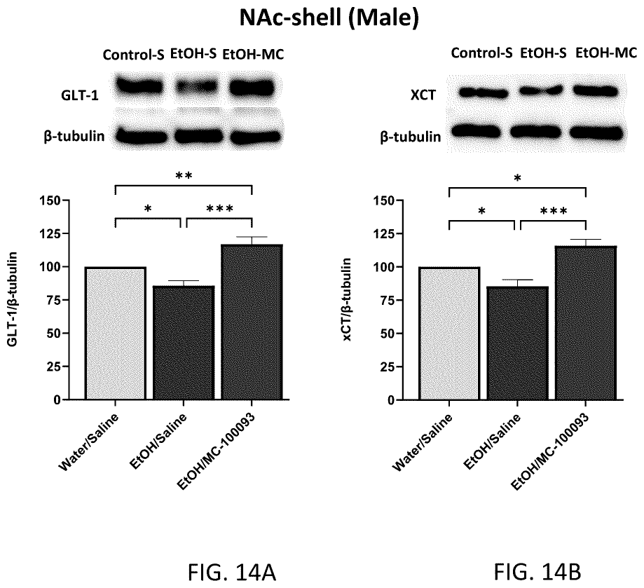

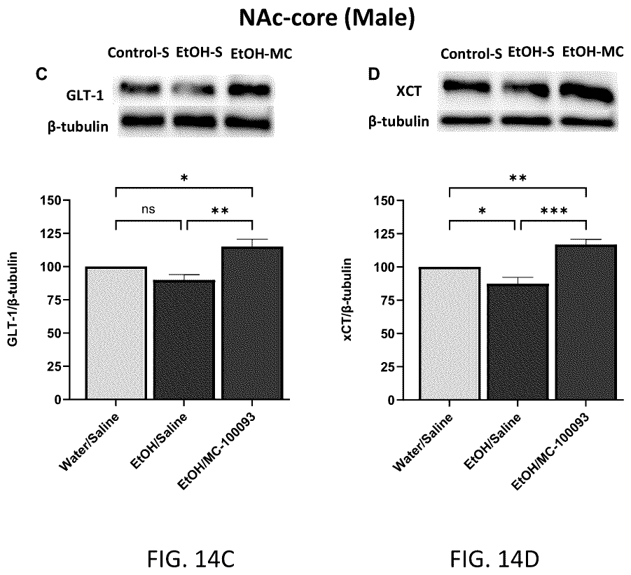

In male P rats, one-way ANOVA showed a significant difference in GLT-1 expression in the NAc-shell among all tested groups (F_{2, 12}=16.68, p=0.0003) (FIG. 14A). A significant decrease in GLT-1 expression in the NAc-shell of the ethanol-saline group as compared to the water-saline group was revealed by Newman-Keuls post-hoc analysis (p<0.05), while its expression was significantly increased in group treated with MC-100093 as compared to the ethanol-saline and water-saline groups (p<0.001, p<0.01 respectively) (FIG. 14A).

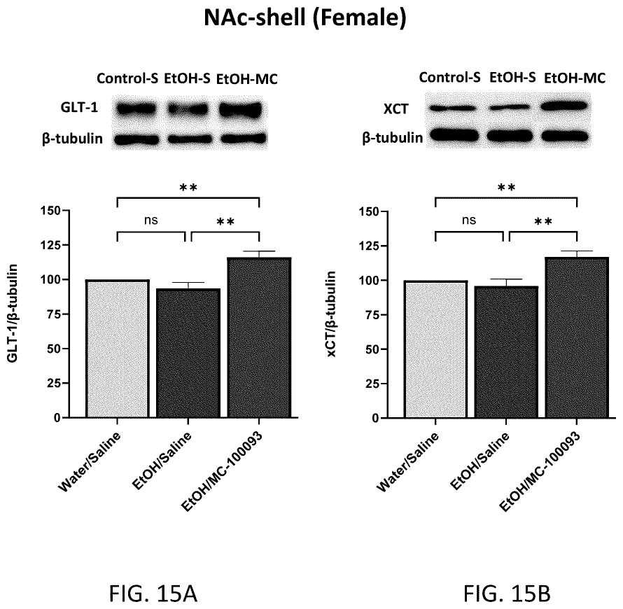

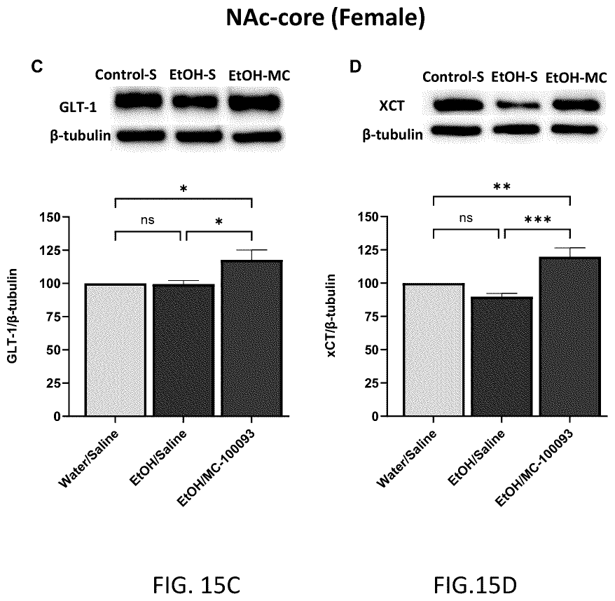

Moreover, there was a significant difference in GLT-1 expression among the three groups in the NAc-shell of female P rats (F_{2, 12}=10.41, p=0.0024) (FIG. 15A). Newman-Keuls post-hoc analysis showed a significant upregulation of GLT-1 expression in the NAc-shell of MC-100093 treated female rats (p<0.01), however, there was no significant reduction in the expression of GLT-1 in the ethanol-saline group compared to the water-saline group (FIG. 15A).

### (8) Effect of MC-100093 on the Expression of xCT in the NAc-Shell of Male and Female P Rats

One-way ANOVA revealed a significant difference in the expression of xCT in the NAc-shell of male P rats among all tested groups (F_{2, 12}=40.16, p=0.0006) (FIG. 14B). Newman-Keuls revealed a significant decrease in the expression of xCT in the NAc-shell of the ethanol-saline group as compared to the water-saline group (p<0.05), while treatment with MC-100093 increased the expression of xCT (p<0.001) (FIG. 14A).

In female P rats, there was a significant difference in xCT expression among the three groups in the NAc-shell (F_{2, 12}=8.526, p=0.0050) (FIG. 15B). Newman-Keuls post-hoc analysis showed a significant upregulation in the expression of xCT in the MC-100093 treated group as compared to ethanol-saline and water-saline groups (p<0.01), whereas no significant changes in the expression of xCT was found in the ethanol-saline group as compared to the water-saline group (FIG. 15B).

### (9) Effect of MC-100093 on the Expression of GLT-1 in the NAc-Core of Male and Female P Rats

In male P rats, one-way ANOVA showed a significant difference in GLT-1 expression in the NAc-core among all tested groups (F_{2, 12}=10.12, p=0.0027) (FIG. 14C). Newman-Keuls post-hoc analysis revealed that GLT-1 expression was significantly increased in group treated with MC-100093 as compared to the ethanol-saline and water-saline groups (p<0.01 and p<0.05 respectively) (FIG. 14C).

There was no difference in the expression of GLT-1 in ethanol-saline group compared to water group. Furthermore, there was a significant difference in GLT-1 expression among the three groups in the NAc-core of female P rats (F_{2, 12}=5.176, p=0.0239) (FIG. 15C). Newman-Keuls post-hoc analysis showed a significant upregulation of GLT-1 expression in the NAc-core of MC-100093-treated female rats (p<0.05) (FIG. 15C). No significant changes were detected between ethanol-saline and water-saline groups in the expression of GLT-1 in NAc-core of female P rats.

### (9) Effect of MC-100093 on the Expression of xCT in the NAc-Core of Male and Female P Rats

One-way ANOVA revealed a significant difference in the expression of xCT in the NAc-core of male P rats among all tested groups (F_{2, 12}=14.52, p<0.0006) (FIG. 14D). Newman-Keuls revealed a significant decrease in the expression of xCT in the NAc-core of the ethanol-saline group as compared to the water-control group (p<0.05), while treatment with MC-100093 reversed the effect of ethanol, and significantly increased the expression of xCT (p<0.001).

In female P rats, there was a significant difference in xCT expression among the three groups in the NAc-core (F_{2, 12}=13.87, p<0.0008) (FIG. 15D). Newman-Keuls post-hoc analysis showed a significant upregulation of xCT expression in ethanol-MC-100093 group as compared to ethanol-saline and water-saline groups (p<0.001 and p<0.01 respectively), whereas no significant changes in the expression of xCT was found in the ethanol-saline group as compared to the water-saline group.

### B-3. Discussion

The present findings demonstrate that treatment with MC-100093 at a dose of 100 mg/kg (i.p.) reduced ethanol intake in male P rats, and that MC-100093 is effective in reducing ethanol consumption in female P rats. These results are in parallel with another study from our laboratory that showed MC-100093 reduced ethanol intake in male P rats with a dose of 50 mg/kg (i.p.). Such a study compared the effect of MC-100093 at a dose of 50 mg/kg in attenuating ethanol drinking to ceftriaxone. The study also compared the effect of MC-100093 and ceftriaxone on GLT-1 and xCT expression in the subregion of NAc shell in male P rats. MC-100093 (50 mg/kg, i.p.) attenuated moderately ethanol intake as compared to ceftriaxone at a dose of 200 mg/kg, i.p. In this study, we focused on testing higher dose of MC-100093 (100 mg/kg, i.p.) as well as we focused on the neurocircuits of PFC and NAc subregions regarding the expression of GLT-1 and xCT. Importantly, we investigated the effects of ethanol consumption and MC-100093 at dose of 100 mg/kg in these neurocircuits to determine for any potential sex difference.

Thus, this study included female P rats and examined the effects of ethanol and MC-100093 in the expression of GLT-1 and xCT in the neurocircuits involving the subregions of mPFC and NAc for comparison with male P rats. MC-100093-induced reduction in ethanol intake was associated with upregulation of GLT-1 expression in the subregions of the mPFC and NAc. The reduction of ethanol drinking was associated with a decrease in ethanol preference starting from Day 1 throughout Day 5 in the ethanol-MC-100093 group as compared to the ethanol-saline group.

It is worth noting that the reduction of ethanol consumption was associated with a significant increase in water intake in the ethanol-MC-100093 group. This increase in water intake might be a compensatory response to the reduction in ethanol consumption during MC-100093 treatment to maintain comparable amount of fluid consumed.

Moreover, MC-100093 treatment showed no effect on the body weight of male and female P rats, which indicates its specificity in reducing ethanol consumption. Nevertheless, it is noteworthy that MC-100093 was associated with a higher attenuation effect on ethanol consumption in male P rats exceeding female P rats. This effect was apparent and continued to increase daily in reducing ethanol intake through the end of the experiment. Whereas MC-100093 attenuation effect in female remained by some means equal across the last three days. Furthermore, a previous study investigated ethanol consumption between male and female P rats, showed that female P rats tend to consume more ethanol than male P rats.

The mesocorticolimbic pathway plays an important role in mediating the rewarding effects of drugs of abuse, including ethanol. mPFC sends and receives glutamatergic projections into the NAc and other brain regions. Therefore, consumption of ethanol is linked to the dysregulation of the glutamatergic pathways in the mesocorticolimbic system, specifically in the mPFC and NAc. Several astroglial glutamate transporters, GLT-1 and xCT, which regulate extracellular glutamate concentrations, are downregulated in the mesocorticolimbic reward pathways following ethanol consumption. Ceftriaxone and other 0-lactam antibiotics exhibited a reduction in ethanol consumption in male P rats and attenuated relapse-like ethanol intake behavior, which was in part associated with an upregulation of GLT-1 and xCT in the NAc and mPFC.

In this study, we examined the expression of GLT-1 in two of the mPFC subregions, specifically, mPFC-IL and mPFC-PL. Results from the present study, for the first time, revealed that five weeks of chronic ethanol consumption was associated with a significant downregulation of GLT-1 expression in the mPFC subregions, mPFC-IL and mPFC-PL in male P rats, and MC-100093 was effective in upregulating GLT-1 expression in these brain subregions. Nonetheless, in female P rats, GLT-1 was downregulated in the mPFC-IL subregions only. However, MC-100093 was associated with the upregulation of GLT-1 expression in both the subregions of the mPFC. It is important to note that rats administered ethanol for four weeks showed a reduction in the expression of GLT-1 in other regions of the PFC. Note, our study is the first to show that ethanol intake for five weeks reduced GLT-1 expression in the subregions of the mPFC such as mPFC-PL and mPFC-IL in male rats, however, downregulation of GLT-1 was observed only in the mPFC-IL in female rats. This indicates possible sex difference in the mPFC-PL with GLT-1 expression. Importantly, xCT expression was downregulated in the subregions of mPFC in both female and male rats. In any cases, MC-100093 upregulated GLT-1 expression in both subregions of the mPFC in either female or male rats.

Following these findings, we also found a downregulation of GLT-1 expression in the NAc-shell but not in the NAc-core following five weeks of chronic ethanol consumption in male P rats, and MC-100093 treatment resulted in attenuating ethanol consumption by upregulating GLT-1. These findings are in line with our studies demonstrated that ethanol drinking for five weeks resulted in the downregulation of GLT-1 expression in the NAc-shell but not in the NAc-core in male P rats. MC-100093 treatment has been effective in upregulating GLT-1 expression in these subregions of the NAc in male P rats. In female P rats, chronic ethanol drinking did not show a significant change in GLT-1 expression in the NAc-shell and NAc-core. Importantly, MC-100093 upregulated GLT-1 in both subregions of the NAc, and this may have attenuated any dysfunctional effect in glutamate homeostasis that might be caused by chronic exposure to ethanol in these subregions of the NAc. Studies are warranted to determine changes in glutamate homeostasis in female and male P rats exposed chronically to ethanol and treated with MC-100093.

The expression of the xCT system is involved in exchanging intracellular glutamate with extracellular cystine. This system can impact mGluR2/3, a presynaptic receptor that regulates the negative feedback for synaptic glutamate release. Hence, we explored the expression of xCT in the subregions of the NAc and mPFC in this study. Thus, chronic ethanol consumption downregulated xCT expression in both the mPFC subregions in male and female P rats. However, chronic ethanol consumption downregulated xCT expression only in the subregions of the NAc in male P rats but not female P rats, which indicate possible sex difference. It is important to note that MC-100093 treatment upregulated xCT expression in both the NAc and the mPFC subregions in male and female P rats.

This study focused on the mPFC-NAc neurocircuits as these projections are involved in the generation of ethanol-cue association and induction of reinstatement to ethanol, which was significantly decreased by the ablation of mPFC neurons projecting to the NAc.

In this study, the effect of chronic ethanol intake on these projections was distinct between the two sex groups within the two different brain subregions. For instance, in male P rats, a significant decrease in the expression of GLT-1 was found in the mPFC-IL and mPFC-PL, and a significant decrease in the expression of GLT-1 was found in the NAc-shell but not the NAc-core. Alternatively, female P rats showed a differential effect of chronic ethanol consumption within the same subregions. The effect of ethanol in the mPFC showed a significant downregulation of GLT-1 expression in the mPFC-IL but not in the mPFC-PL. There were no changes in the expression of GLT-1 in the NAc-shell or NAc-core.

To the best to our knowledge, there is less known about the effects of exposure to ethanol on the expression of GLT-1 and xCT in the neurocircuits involving the subregions of NAc and mPFC in females. We suggest that the lack of effects of ethanol intake on the expression of GLT-1 and xCT in the NAc subregions may be associated with changes in sex hormones. Future studies are warranted to use ovariectomized female rats to determine whether hormonal changes can have an effect in the modulation of GLT-1 and xCT expression in model of chronic ethanol exposure in the subregions of NAc.

In conclusion, the results from this study indicate that treatment with MC-100093 at a dose of 100 mg/kg (i.p.) decreased ethanol consumption in male and female P rats, partly by increasing GLT-1 and xCT expression in the mPFC and NAc subregions. The present study supports the rationale that MC-100093 treatment reduced ethanol intake by restoring basal extracellular glutamate concentrations in the mesocorticolimbic neuronal circuits partly through upregulation of GLT-1 and xCT expression. Further studies are needed to determine the molecular mechanisms of MC-100093 and investigate dose-dependent effects in ethanol intake and the expression of these astrocytic glutamate transporters in brains of both male and female rats.

### C. Effects of Compound on Neuroinflammatory and Neurotrophic Biomarkers

We have studied effects of an exemplary compound, MC-100093, which is a novel GLT-1 modulator, on neuroinflammatory and neurotrophic biomarkers in mesocorticolimbic brain regions of male alcohol preferring rats exposed chronically to ethanol.

Chronic ethanol consumption can lead to increased extracellular glutamate concentrations in key reward brain regions, such as medial prefrontal cortex (mPFC) and nucleus accumbens (NAc), and consequently leading to oxidative stress and neuroinflammation. Studies from our lab tested β-lactam antibiotics and novel β-lactam non-antibiotic, MC-100093, and showed these β-lactam upregulated the major astrocytic glutamate transporter, GLT-1, and consequently reduced ethanol intake and normalized glutamate homeostasis.

This present study tested the effects of novel synthetic β-lactam non-antibiotic drug, MC-100093, in chronic ethanol intake and neuroinflammatory and trophic factors in subregions of the NAc (NAc core and shell) and mPFC (Prelimbic, PL; and Infralimbic, IL) of male P rats. MC-100093 treatment reduced ethanol intake after 5-week drinking regimen. Importantly, MC-100093 attenuated ethanol-induced downregulation of brain derived neurotrophic factor (BDNF) expression in these brain regions.

In addition, MC-100093 attenuated ethanol-induced upregulation of pro-inflammatory cytokines such as TNF-a and HMGB1 in all these brain regions. Furthermore, MC-100093 treatment attenuated ethanol-induced increase in receptor for advanced glycation end products (RAGE) in these brain regions. MC-100093 prevented neuroinflammation caused by ethanol intake as well as increased neurotrophic factor in mesocorticolimbic brain regions. MC-100093 treatment reduced ethanol intake and this behavioral effect was associated with attenuation of reduced trophic factors and increased pro-inflammatory factors. MC-100093 is considered a small molecule that may have potential therapeutic effects for the treatment of the effects of chronic exposure to ethanol.

Chronic alcohol consumption alters several neurotransmitters, including glutamate. This may cause an increase in extracellular glutamate concentrations in key reward brain regions, including the dorsomedial prefrontal cortex (dmPFC) and nucleus accumbens (NAc), which can lead to oxidative stress and neuroinflammation. Chronic ethanol intake induced inflammation and consequently apoptosis of neurons and glial cells. The NAc has been found to be a mediator of the reward system in animal models and contributes to the mechanism of ethanol drinking alcohol.

Studies performed in our laboratory have determined that beta-lactam antibiotics such as ceftriaxone and others are beneficial in decreasing ethanol consumption in alcohol preferring (P) rats. Among beta-lactams, ceftriaxone has been one the drugs to reduce ethanol intake and this effect was associated with normalization and upregulation of GLT-1.

The study from our laboratory tested a novel beta-lactam, MC-100093, which does not have any antibiotic action, and the drug was found to reduce ethanol intake. The decreased in ethanol intake was found to be associated with the normalization of GLT-1 in the NAc. It is important to note that the pharmacokinetics of MC-100093 have been studied using in vitro and in vivo paradigms. This study showed that MC-100093 had high aqueous solubility as well as displayed a high stability in rodent and human liver microsomes in vitro. The drug has low binding to plasma protein and low partitioning in lipid membranes. In vivo pharmacokinetic studies found that MC-100093 displayed some bioavailability following oral administration. There is no oxidative metabolism which points to the fact that the bioavailability following intraperitoneal administration is similar to the bioavailability of oral dosing. In vivo half-life was also found to be 4.2. hours via the intraperitoneal route and 5.26 hours via the oral route.

In this present study, we have investigated whether MC-100093 would attenuate ethanol-induced increases in neuroinflammatory factors such as tumor necrosis factor (TNF-α), receptor for advanced glycation end products (RAGE) and high mobility group box-1 (HMGβ-1) and alteration of neurotrophic factor such as brain derived neurotrophic factor (BDNF). This study focused on investigating for potential changes in the expression of BDNF in the subregions of the NAc (NAc core and shell) and mPFC (Prelimbic, PL; and Infralimbic, IL) as there is association between BDNF signaling and glutamate release. BDNF is a trophic factor involved in neuronal survival and growth, and substances of abuse can alter its expression. The NAc shell mediates ethanol extinction and is involved in signaling satiety. BDNF overexpression in the NAc has been found to reduce withdrawal symptoms and craving behavior in a rat model.

We then focused on investigating HMGβ-1 as inflammatory marker, which stimulates several receptors, and RAGE is one of these target receptors. HMGβ-1 is a pro-inflammatory-like cytokine that is released due to increase oxidative stress. It has been observed to be overexpressed in several neuroinflammatory conditions, such as Alzheimer's disease and Parkinson's disease. RAGE is a multi-ligand receptor that binds to several different ligands, including HMGβ-1. Interaction between RAGE and its ligands activates several cellular processes, including neuroinflammation. The tumor necrosis superfamily of cytokines plays a role in many chronic inflammatory and neurodegenerative conditions, and TNF-α is a key mediator of neuroinflammation. Alcohol-induced neuroinflammation is mediated by pro-inflammatory cytokines such as TNF-α. HMGβ-1 and TNF-α have been suggested to be involved in glutamate neurotoxicity. Thus, this study focused on these signaling inflammatory pathways in association with BDNF as trophic factor in the subregions of the NAc (NAc core and shell) and mPFC (Prelimbic, PL; and Infralimbic, IL) of male P rats exposed to free choice of water and ethanol (15% and 30%, v/v) for 6 weeks and treated with either vehicle saline or MC-100093.

### C-1. Materials and Methods

### Animal Model

Male P rats were obtained from Indiana University School of Medicine (Indianapolis, IN, USA). P rats were ˜90 days old at the beginning of the drinking paradigms. P rats were used in this study as they fit the necessary criteria of ethanol preference. P rats may consume freely ethanol (15% and 30%, v/v) and achieve a pharmacologically blood alcohol concentration (BACs) of 50-200 mg % as shown in previous studies from Indiana University. Rats were housed in a room kept at 21° C. on a 12/12-hour light/dark cycle and had free access to water and food. All experimental procedures were approved by the Institutional Animal Care and Use Committee (IACUC) at University of Toledo under protocol #400160, in accordance with all guidelines of IACUC of the National Institute of Health, and the Guide for the Care and Use of Laboratory Animals.

### Ethanol-Intake Model:

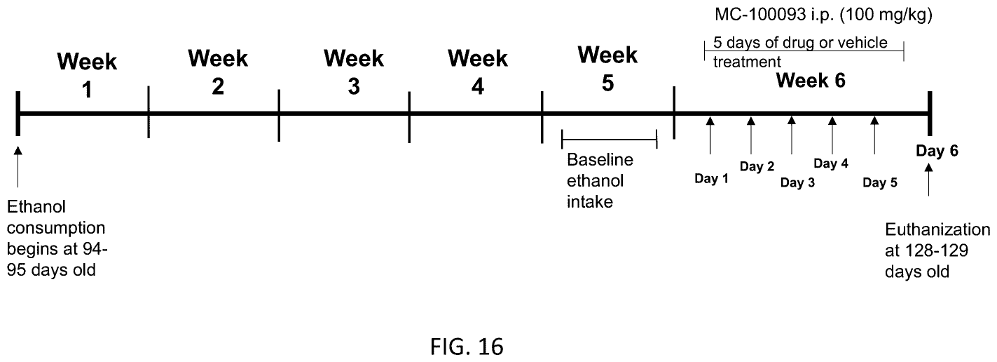

Rats were assigned to three groups prior to the start of the experiments based on their weights, and the average weights of each group were similar between all groups. Male P rats were divided into three groups: a) Control/water group was exposed to water for five weeks and received saline vehicle injection (i.p.) every 24 hours during Week 6 for a total of five days; b) Ethanol/saline group was exposed to free choice of ethanol (15% and 30%, v/v) as well as water for 5 weeks and received saline vehicle (i.p.) every 24 hours during Week 6 for a total of five days; c) Ethanol/MC-100093 group was exposed to free choice of ethanol (15% and 30%, v/v) as well as water for 5 weeks and received MC-100093 (100 mg/kg, i.p.) every 24 hours during Week 6 for 5 days (FIG. 16).

The positioning of the three bottles were randomly interchanged at the beginning of each week to avoid preference amongst the animals. Measurements of ethanol intake occurred by taking the weight in grams. After Week 3, measurements of ethanol intake occurred every other day through Week 4. Ethanol/saline (n=5 per group) and ethanol/MC-100093 (n=6 per group) groups were exposed to free choice water, 15% ethanol and 30% ethanol (v/v) for 24 hours over 5 weeks.

The baseline was calculated by taking the average of the prior 5 weeks of water and ethanol consumption as rat weight. The water/saline control group and the Ethanol/saline groups received saline for 5 days starting on Day 1 to Day 5 on week 6. Ethanol/MC-100093 group received an equivolume i.p. injection of MC-100093 (100 mg/kg) for 5 days starting on Day 1 to Day 5 of Week 6. As weights of the bottles were recorded, the measurements were converted using g of ethanol intake/kg of body weight/day formula to determine ethanol consumption in g/kg of body weight. Rats with an average ethanol intake less than 4 g/kg/day were excluded from the study as this measurement did not follow the criteria for the development of ethanol dependence. Rats were then euthanized by CO₂ inhalation followed by decapitation on Day 6 of Week 6.

MC-100093 was synthesized and characterized at Temple University, Moulder Center for Drug Discovery Research, Philadelphia, PA.

### Brain Tissue Extraction

All P rats were euthanized by CO₂ inhalation and decapitated with a guillotine 24 hours after the final i.p. injection was administered on Day 6 of Week 6. Brains were removed and immediately frozen on dry ice and stored at −80° C. The mPFC subregions (infralimbic cortex and prelimbic cortex) and NAc subregions (core and shell) were dissected using a cryostat machine kept at −20° C. Surgical blades were used to dissect all brain regions following visualized landmarks using the stereotaxic coordinates provided by Paxinos and colleague's rat brain stereotactic atlas. All brain regions were stored in −80° C. Western Blot analyses.

### Western Blot Procedure:

Western blot was performed to determine the expression of BDNF, TNF-α, RAGE, HMGβ-1, and β-tubulin in the subregions of mPFC and NAc as preformed in previous studies.

Brain samples were lysed with a lysing buffer containing protease inhibitors. The amount of protein in each sample was quantified using a detergent compatible protein assay (Bio-Rad, Hercules, CA, USA). Polyacrylamide gels (10%) were loaded with the lysate mixture, and proteins were separated using gel electrophoresis for 1 hour. Proteins were then transferred from the gels onto Polyvinylidene difluoride (PVDF) membranes (BioRad). After transfer, membranes were incubated for 30 minutes in a mixture of nonfat milk in 1×Tris-buffered saline with Tween-20 (TBST) at room temperature. Membranes were then incubated at 4° C. for 24 hours with primary antibodies: anti-rabbit BDNF (1:1000, Abeam, #ab108319), anti-rabbit TNF-α (1:500, Abeam, #ab9739), anti-rabbit HMGβ-1 (1:500, Abeam, #ab18256), and anti-rabbit RAGE (1:1000, Abeam, #ab37407). Anti-mouse-β-tubulin (1:1000, Cell Signaling Technology, #D71G9) was used as a loading control antibody. After incubating for 24 hours, secondary antibody such as donkey anti-rabbit (1:4000, Invitrogen, #31458) or anti-mouse (1:4000, Cell Signaling, #7076S) was added to the membrane for 1 hour at room temperature. The membranes were then washed with 1×TBST three times for 5 minutes each and then dried. Membranes were then incubated in chemiluminescent reagents (Super Signal West Pico, Pierce Inc.) for 2 minutes. Blot images were developed using ChemiDoc. Imaging System and ImageJ software version 1.53a were used to quantify and analyze the expression of BDNF, TNF-α, RAGE, HMGβ-1, and β-tubulin.

The data for the water/saline control group were expressed as 100% based on the values corresponding to the ethanol/saline and ethanol/MC-100093 groups for each set of blotting. Thus, the expression of selected proteins for each group (ethanol/saline group or ethanol/MC-100093 group) was calculated relative to the water/saline control group being set to 100%.

### Statistical Analysis

Statistical analyses were performed using GraphPad Prism Version 10. Two-way repeated measures ANOVA followed by Bonferroni post-hoc tests were used to compare water and ethanol intake, and body weight, amongst water group, ethanol/saline group and ethanol/MC-100093 group. A one-way ANOVA test was performed to compare the expression of the studied proteins in selected brain regions in water/saline, ethanol/saline and ethanol/MC-100093 groups. Statistical significance was determined by a p-value of or less than 0.05.

### C-2. Results

### (1) Effects of MC-100093 on Ethanol and Water Consumption as Well as Body Weight in P Rats

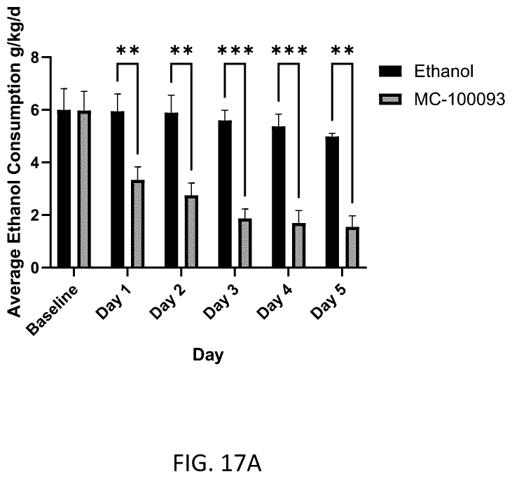

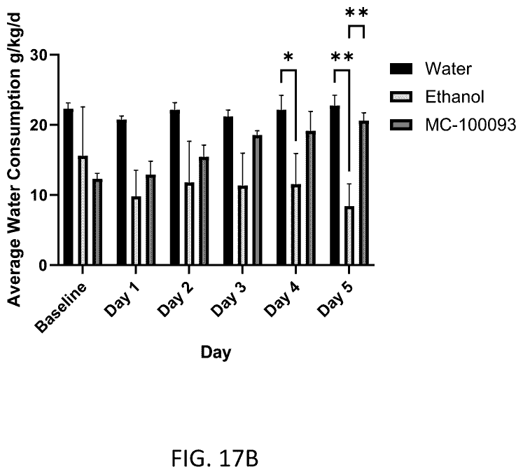

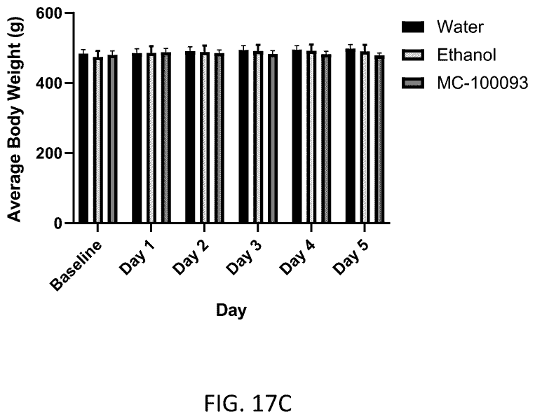

Two-way repeated measures ANOVA analysis revealed a significant main effect of Day on ethanol intake, [F (1,61)=68.81, p<0.0001] and a significant Treatment by Day interaction, [F 5,61)=3.312. p<0.0104](FIG. 17A). Bonferroni post-hoc test revealed a significant decrease in ethanol intake on days 1 and 2 in rats treated with MC-100093 as compared to rats treated with saline vehicle (p<0.01). There was also a significant decrease in ethanol consumption in rats treated with MC-100093 as compared to rats treated with saline vehicle (p<0.001) on days 3 and 4 and a significance decrease in ethanol intake on day 5 in rats treated with MC-100093 as compared to rats treated with saline vehicle (p<0.01).

Two-way repeated measures ANOVA analysis revealed no significant main effect of Day on water intake [F(5,83)=0.3422, p=0.6549] as well as no significant Treatment dose by Day interaction on water intake [F(10,83)=0.7722, p=0.8858](FIG. 17B). Bonferroni post-hoc test revealed a significant decrease in water intake on day 4 (p<0.05) and day 5 (p<0.01) in rats treated with saline vehicle as compared to control group exposed to water only and treated with saline vehicle. There was a significant increase in water intake in rats treated with MC-100093 and exposed to ethanol on day 5 (p<0.01) (FIG. 17B).

Two-way repeated measures ANOVA followed by Bonferroni post-hoc tests did not reveal any main effect of Day on average body weight [F(5,96)=0.2161, p=0.9549], and there was no significant difference found between the day and average body weight (FIG. 17C). There was no significant effect of Treatment by Day interaction on body weight [F (10,96)=0.1016, p=0.9998].

(2) Effects of MC-100093 and Chronic Ethanol Consumption on BDNF Expression in the Subregions of mPFC and NAc

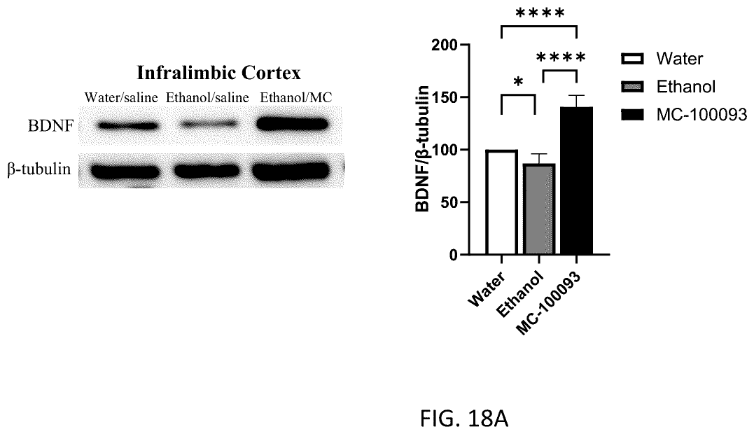

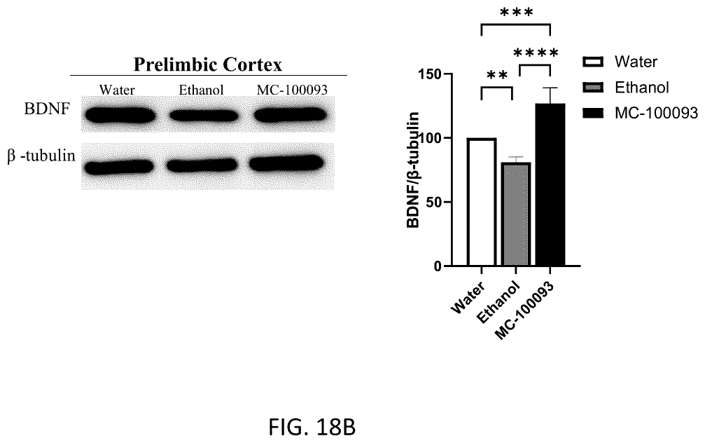

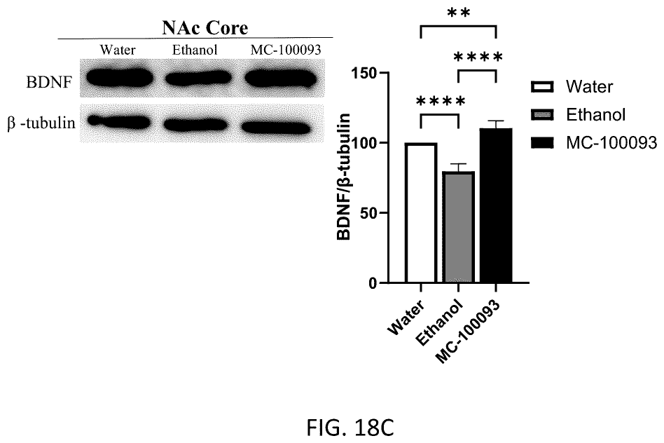

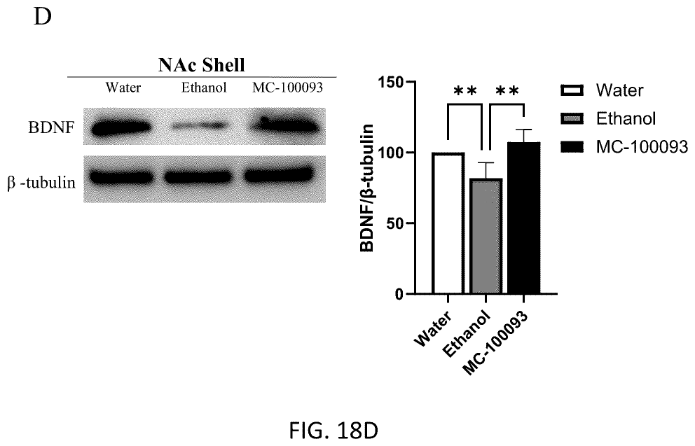

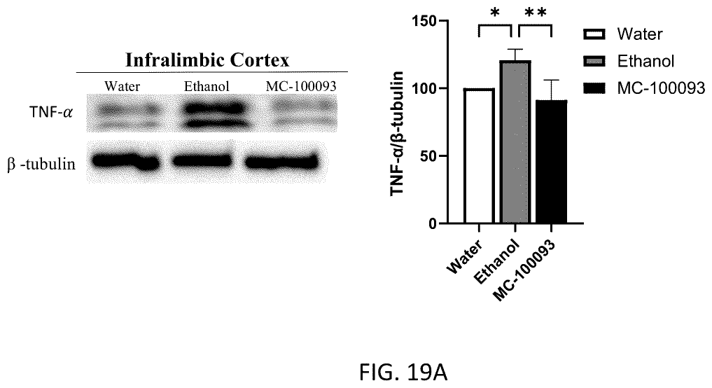

One-way ANOVA revealed a significant difference in BDNF expression in the subregion of mPFC, IL, among all tested groups [F(2,11)=57.01, p<0.0001]. Newman-Keuls test showed a significant increase (40%) in BDNF expression in the ethanol/MC-100093 group as compared to water/saline and ethanol/saline groups (p<0.0001) as well as a significant decrease (13%) in BDNF expression in the ethanol/saline group as compared to water/saline group (p<0.05) (FIG. 18A).

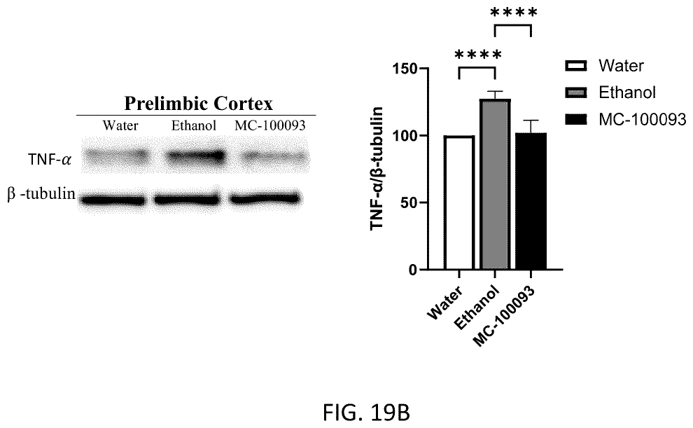

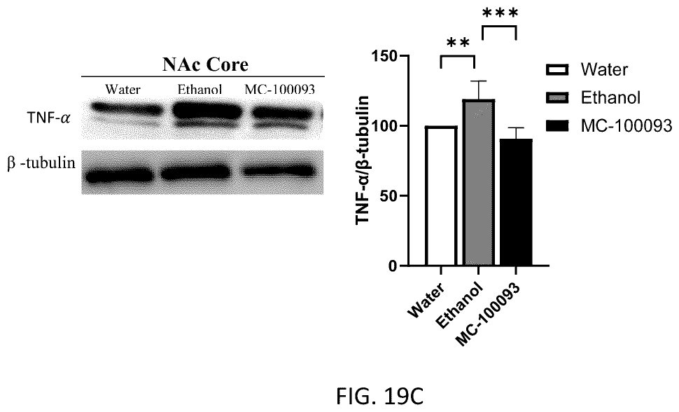

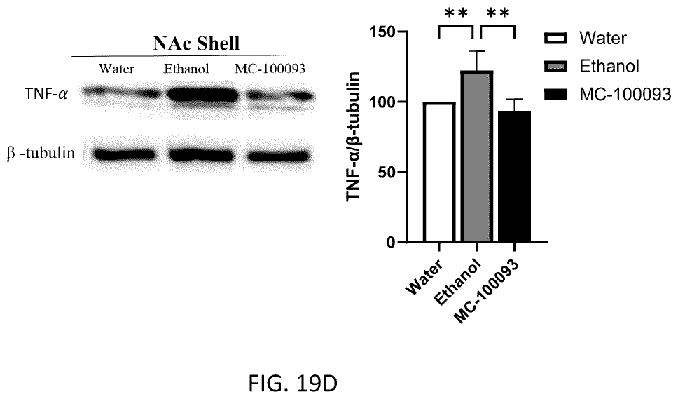

In the subregion of mPFC, PL, there was a significant difference in BDNF expression among all tested groups [F(2,10)=41.96, p<0.0001]. Newman-Keuls test showed a significant increase (27%) in BNDF expression in the ethanol/MC-100093 group as compared to the water/saline group (p<0.001) and the ethanol/saline group (p<0.0001) (FIG. 19B). There was a significant decrease (18%) in BDNF expression in the PL in the ethanol/saline group as compared to the water/saline group (p<0.01) (FIG. 18B).

Regarding the subregions of the NAc, there was significance difference in the expression of BDNF in NAc core among all tested groups [F(2,9)=55.86, p<0.0001]. Newman-Keuls test showed a significant increase (11%) in BDNF expression in the NAc core of rats treated with MC-1000093 as compared to the water/saline group (p<0.01) and ethanol/saline group (p<0.0001), as well as a significant decrease (21%) in BDNF expression in the ethanol/saline group compared to the water/saline group (FIG. 18C).

There was also a significant decrease in BDNF expression in the ethanol/saline group as compared to the water/saline group (p<0.0001). Furthermore, there was also significance difference in the expression of BDNF in NAc shell among all tested groups [F(2,10)=11.78, p<0.05]. Newman-Keuls test showed a significant increase in BDNF expression in the ethanol/MC-100093 group as compared to the ethanol/saline group (p<0.01) (FIG. 19D). There was also a significant decrease (18%) in BDNF expression in the NAc shell in the ethanol/saline group as compared to the water/saline group (p<0.01) (FIG. 18D).

(3) Effects of MC-100093 and Chronic Ethanol Consumption on TNF-α Expression in the Subregions of mPFC and NAc

One-way ANOVA revealed a significant difference in TNF-α expression in the mPFC subregion, IL, among all tested groups [F(2,9)=8.732, p<0.01]. Newman-Keuls test showed a significant increase (21%) in TNF-α expression in the IL in the ethanol/saline as compared to the water/saline group (p<0.05), and MC-100093 treatment decreased significantly TNF-α expression in the IL as compared to ethanol/saline group (p<0.01) (FIG. 19A).

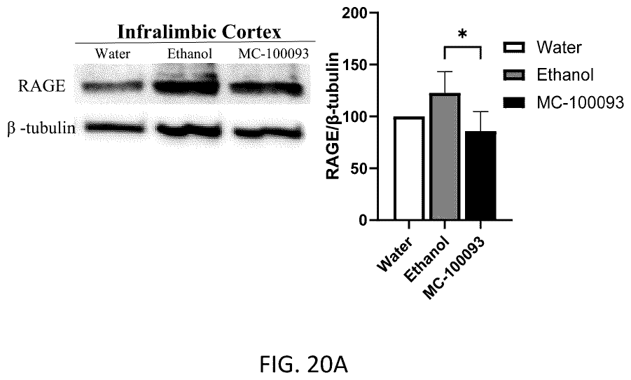

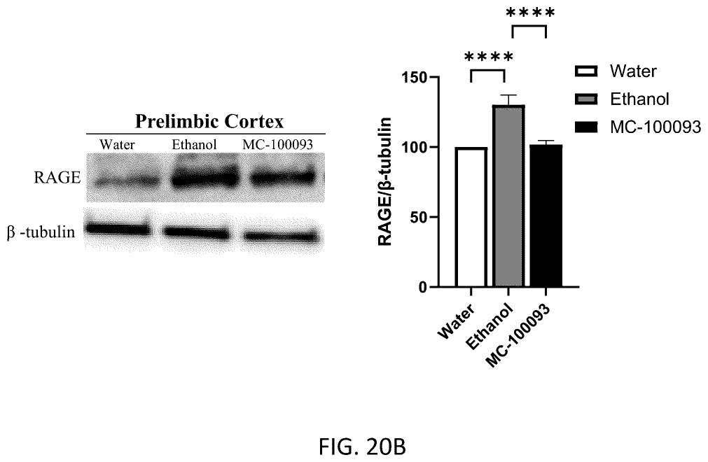

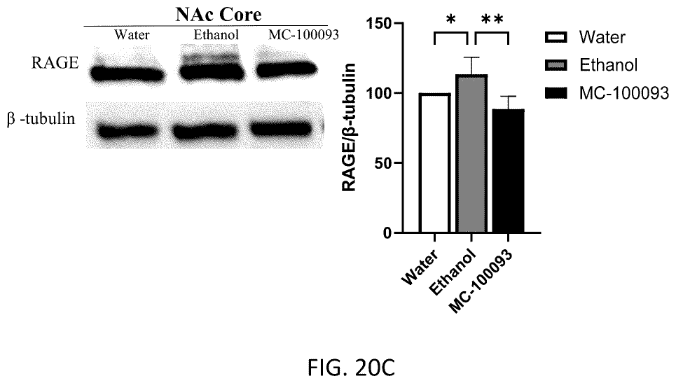

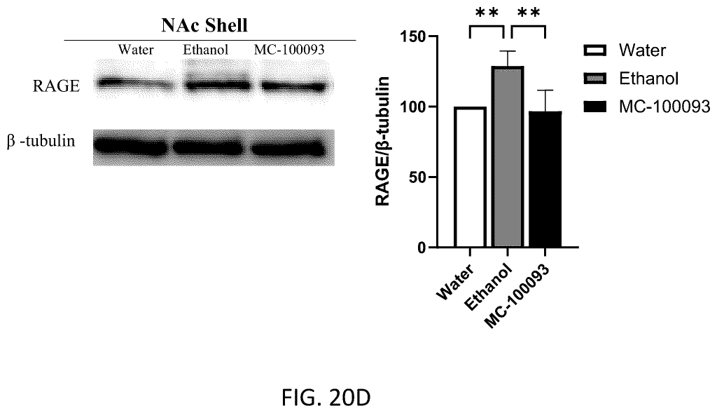

Regarding the mPFC subregion, PL, there was also a significant difference in the TNF-α expression among all groups [F(2,10)=36.26, p<0.0001]. Newman-Keuls test revealed a significant increase (28%) in TNF-α expression in the PL in the ethanol/saline group as compared to the water/saline group (p<0.0001) (FIG. 20B), and MC-100093 treatment significantly decreased the expression of TNF-α as comparing to the ethanol/saline group (p<0.0001) (FIG. 19B).

Regarding the subregions of the NAc, one-way ANOVA revealed a significant difference in the expression of TNF-α in the NAc core among all groups [F(2,11)=13.62, p<0.01]. Newman-Keuls test showed a significant increase (20%) in the TNF-α expression in the NAc core in the ethanol/saline group as compared to the water/saline group (p<0.01) (FIG. 19C). MC-100093 treatment decreased significantly TNF-α expression in the NAc core as compared to the ethanol/saline group (p<0.001). In the NAc shell, there was a significant difference in the TNF-α expression among all groups [F(2,11)=11.88, p<0.01]. Newman-Keuls test showed a significant increase (23%) in the TNF-α expression in the NAc shell in the ethanol/saline group as compared to the water/saline group (p<0.01) (FIG. 19D). Similarly, MC-100093 treatment decreased TNF-α expression in the NAc shell as compared to the ethanol/saline group (p<0.01) (FIG. 19D).

(4) Effects of MC-100093 and Chronic Ethanol Consumption on RAGE Expression in the Subregions of mPFC and NAc

In the IL subregion of mPFC, there was a significant difference in the expression of RAGE among all groups [F(2,9)=5.558, p<0.05]. Newman-Keuls test revealed that MC-100093 treatment decreased (14%) the expression of RAGE in the IL as compared to the ethanol/saline group (p<0.05) (FIG. 20A).

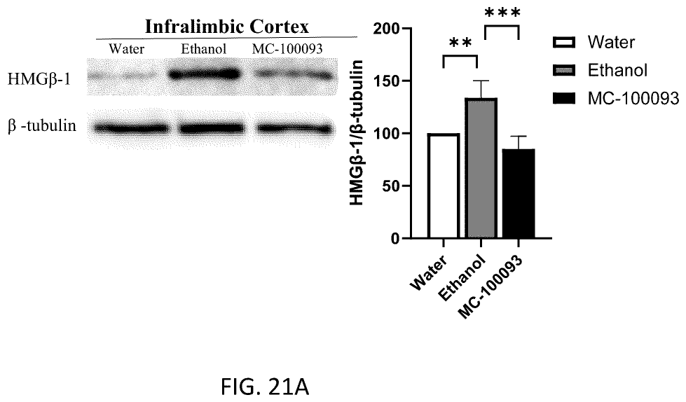

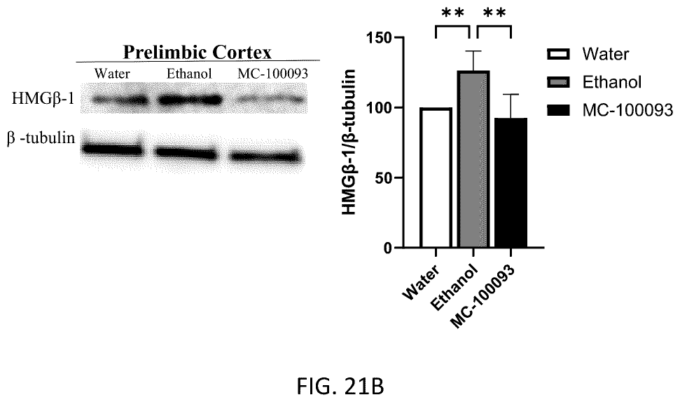

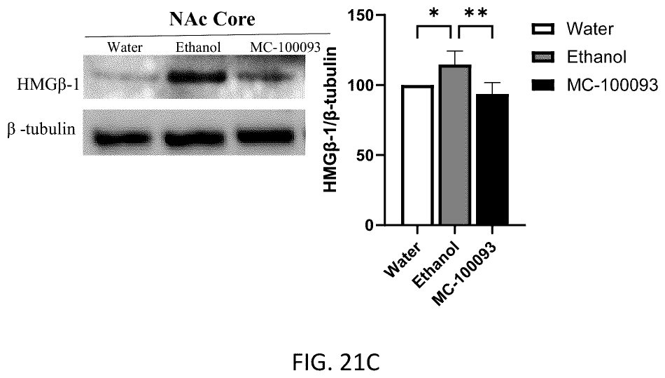

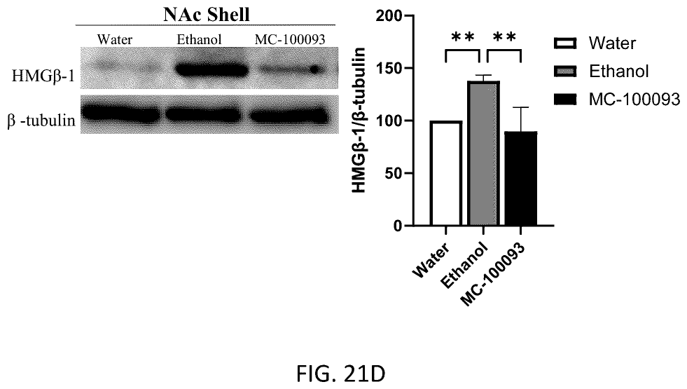

In the PL, one-way ANOVA revealed significant difference among all groups [F(2,9)=71.40, p<0.0001]. Newman-Keuls test revealed a significant increase (30%) in the RAGE expression in the PL in the ethanol/saline group as compared to the water/saline group (p<0.0001) (FIG. 21B). MC-100093 treatment decreased RAGE expression in the PL as compared to the ethanol/saline group (p<0.0001) (FIG. 20B).

In the NAc core, there was a significant difference among all groups [F(2,11)=9.679, p<0.01]. Neuman-Keuls test revealed a significant increase (14%) in the RAGE expression in the NAc core in the ethanol/saline group as compared to the water/saline group (p<0.05) (FIG. 21C). MC-100093 treatment decreased (12%) RAGE expression as compared to the ethanol/saline group (p<0.01) (FIG. 20C). In the NAc shell, there was a significant difference among all groups [F(2,9)=13.90, p<0.01)].

Newman-Keuls test revealed a significant increase (30%) in RAGE expression in the NAc shell in the ethanol/saline group as compared to the water/saline group (p<0.01) (FIG. 21D). MC-100093 treatment decreased RAGE expression as compared to the ethanol/saline group (p<0.01). (FIG. 20D).

(5) Effects of MC-100093 and Chronic Ethanol Consumption on HMGβ-1 Expression in the Subregions of mPFC and NAc

One-way ANOVA analysis revealed a significant difference in the HMGβ-1 expression in the mPFC subregion, IL, among all groups [F(2,8)=18.48, p<0.01)]. There was a significant increase (35%) in HMGβ-1 expression in the ethanol/saline group as compared to the water/saline group (p<0.01) (FIG. 21A).

MC-100093 treatment decreased HMGβ-1 expression in the IL as compared to the ethanol/saline group (p<0.01) (FIG. 21A). Furthermore, there was a significant difference in the expression of HMGβ-1 in PL among all groups [F_{(2,9)}=9.358, p<0.01]. There was a significant increase (27%) in HMGβ-1 expression in the ethanol/saline group as compared to the water/saline group (p<0.01) (FIG. 21B). MC-100093 treatment decreased the expression of HMGβ-1 in the PL as compared to the ethanol/saline group (p<0.01) (FIG. 21B). Regarding the subregions of the NAc, there was a significant difference in the expression of HMGβ-1 in the NAc core among all groups [F (2,8)=8.929, p<0.01)].

Neuman-Keuls test showed a significant increase (15%) in the expression of HMGβ-1 in the NAc core in the ethanol/saline group as compared to the water/saline group (p<0.05) (FIG. 21C). MC-100093 treatment decreased the expression of HMGβ-1 in the NAc core as compared to the ethanol/saline group (p<0.01) (FIG. 21C). One-way ANOVA revealed a significant difference in the expression of HMGβ-1 in the NAc Shell among all groups [F(2,9)=11.79, p<0.01].

Neuman-Keuls tested revealed a significant increase (38%) in the expression of HMGβ-1 in the NAc shell as compared to the water/saline group (p<0.01) (FIG. 21D). MC-100093 treatment decreased the expression of HMGβ-1 in the NAc shell as compared to the ethanol group (p<0.01) (FIG. 21D).

### B-3. Discussion

This study revealed that treatment with novel beta-lactam, MC-100093, attenuated the effects of ethanol-induced alterations in several neuroinflammatory and trophic factors in brains of male P rats. In this study, we only tested male P rats to determine the effects of our novel beta-lactam, MC-100093, in ethanol intake and neuroinflammatory and neurotrophic biomarkers. This study was focused in identifying MC-100093 as potential drug target for potential therapeutic effects in ethanol dependence animal model. Further studies are on-going for testing the effects of MC-100093 in female P rats to determine whether this drug has an effect in ethanol intake and neuroinflammatory and trophic factors. In the present study, using male P rats, we found that MC-100093 treatment reduced ethanol intake in P rats staring 24 hours after the first injection of the drug through the five days regimen.

In addition, MC-100093 showed an effect on water consumption. There was a trend of an increase in water consumption throughout the five days of treatment. On the fifth day, the ethanol/MC-100093 group drank significantly more water than the ethanol/saline group. MC-100093 at a higher dose (100 mg/kg, i.p.) has more effective as compared to the dose of MC-100093 (50 mg/kg, i.p.) tested in recent study from our lab. Thus, MC-100093 (100 mg/kg, i.p.) showed a robust effect in ethanol intake in this present study.

Chronic ethanol consumption for 6 weeks decreased the expression of BDNF in the subregions of mPFC (IL and PL) and NAc (core and shell). In addition, chronic exposure to ethanol upregulation of TNF-α, RAGE and HMGβ1 in the IL, NAc core, and NAc shell. Importantly, MC-100093 treatment during Week 6 reduced ethanol intake and this effect was associated with attenuation of ethanol-induced decrease in BDNF expression and increase in TNF-α, RAGE and HMGβ1. MC-100093 reduced ethanol intake in P rats at higher dose (100 mg/kg). This latter study showed also that reduction in ethanol intake with MC-100093 was associated with upregulation of GLT-1 and xCT in the NAc.

In this present study, MC-100093 treatment attenuated ethanol-induced downregulation of BDNF expression in the subregions of NAc and mPFC. It is important to note that BDNF is critical on modulating vesicular glutamate transporters and glutamate itself may alter BDNF expression, which suggest the interactive role between glutamate and BDNF in the regulation of synaptic transmission. MC-100093 attenuates ethanol-induced downregulation of GLT-1 expression in central reward brain region such as NAc, thus, normalizing glutamate homeostasis an effect that is suggested to be associated with normalization of BDNF expression.

It is important to note that the effect of ethanol exposure in the expression of BDNF depends on the ethanol exposure regimen, timing, age of animals and the targeted brain regions. For example, acute ethanol exposure increased BDNF expression; however, withdrawal after long term exposure to ethanol induced downregulation of BDNF in the central amygdala and medial amygdala. This suggests the downregulatory effect of BDNF with exposure to ethanol.

However, our studies showed that chronic ethanol intake increased BDNF expression only in NAc shell. This differential effect of ethanol exposure in BDNF expression in this brain region is probably due to the fact P rats consumed a higher amount of ethanol (˜10 g/kg/24 hr) during the five days of i.p. injection of MC-100093 performed on Week 6 of either saline vehicle or ceftriaxone as compared to our present study where rats drank less than 6 g/kg/24 hr over the five days i.p. injection of saline vehicle or MC-100093 on Week 6. The effect of high consumption of ethanol may lead to neuroadaptive mechanism involving BDNF expression. In addition, acute exposure to ethanol increased BDNF expression in the dorsal striatum; however, exposure to ethanol for six weeks decreased BDNF expression in the cortex of mice an effect similar to our present finding. It is important to note that BDNF polymorphism might be associated with increase in ethanol consumption and development of ethanol dependence in mice. Additionally, study demonstrated that microRNA-induced reduction of BDNF expression in the mPFC was associated with escalated ethanol intake in Wistar rats. Further studies are warranted to investigate the amount consumed of ethanol and duration of ethanol exposure on the expression of BDNF in several reward brain regions.

Our present study further investigated the effects of chronic exposure to ethanol and MC-100093, beta-lactam known to upregulate GLT-1, in neuroinflammatory factor such as TNF-α. This study revealed that chronic ethanol exposure increased TNF-α expression in the subregions of mPFC and NAc. This is in accordance with our studies demonstrated that chronic exposure to ethanol for six weeks increased the expression of TNF-α in the NAc shell of male high alcohol drinking (HAD1) rats. Additionally, binge ethanol consumption for only ten days followed by one day withdrawal period increased TNF-α in the brain. Other studies revealed that chronic ethanol exposure increased TNF-α expression in the cerebral cortex of both mouse genders. Importantly, since we have been successful in attenuating the effect of chronic ethanol-induced increase in TNF-α expression in the NAc of HAD1 with beta-lactams ampicillin/sulbactam, known to upregulate GLT-1, we tested our novel drug, MC-100093, which upregulates GLT-1 expression in the brain. Indeed, MC-100093 has been effective in normalizing TNF-α expression against the effect of chronic ethanol exposure, and this is in accordance with our previous study using beta-lactam antibiotics, ampicillin/sulbactam.

Furthermore, this study revealed that chronic ethanol exposure increased the expression of HMGβ-1 and RAGE in the subregions of mPFC and NAc. It is important to note that HMGβ-1 can activate RAGE, which in turn activates the NF-KB signaling pathway to increase TNF-α. In postmortem human brains from AUD patients, RAGE was found to be increased. In addition, studies demonstrated that chronic ethanol consumption resulted in an increase of NF-KB pathway, which was associated with increase in several cytokines in the rat brain, including HMGβ-1. Furthermore, binge ethanol drinking for ten days increased HMGβ-1 expression in the brain of mice. Other studies reported that chronic ethanol exposure for five weeks increased HMGβ-1 as well as its receptor, RAGE, in the cerebellum. Additionally, previous studies from our laboratory demonstrated that chronic ethanol intake for six weeks increased both HMGβ-1 and RAGE in the NAc of HAD1 rats. Similar to TNF-α, this latter study revealed that beta-lactams, ampicillin/sulbactam known to upregulate GLT-1, attenuated the effect of ethanol-induced increase in both HMGβ-1 and RAGE in the NAc of HAD1 rats. This is in accordance with our present study demonstrated that MC-100093, GLT-1 upregulator, attenuated ethanol-induced increase in both HMGβ-1 and RAGE in the subregions of mPFC and NAc of P rats. It is important to note that increase in the expression of HMGβ-1 is critical in the glutamate neurotoxicity as suggested by other studies. Thus, ethanol-induced downregulation of GLT-1 is associated with increased extracellular glutamate concentration, which might associated with increased in HMGβ-1 expression in target brain regions of the reward circuit. Thus, modulating GLT-1 expression with MC-100093 can lead to regulation of glutamate homeostasis and consequently attenuation of ethanol-induced neuroinflammation.

Based on the results above (Section C), MC-100093 treatment attenuated chronic ethanol consumption in male P rats. Furthermore, chronic ethanol consumption reduced BDNF expression in the subregions of the mPFC and NAc, increased pro-inflammatory cytokines such as TNF-α and HMGB1. In addition, chronic ethanol intake increased the expression of RAGE as mediator protein of inflammatory response. The attenuating effects of MC-100093 in the expression of BDNF and pro-inflammatory cytokines and mediator protein of inflammation might be associated with normalization of glutamate homeostasis as the drug is known to normalize the expression of astrocytic glutamate transporter such as GLT-1. This study presents a novel beta-lactam, MC-100093, that has the potential to attenuate ethanol intake in chronic ethanol regimen, and this attenuating effect of ethanol consumption was associated with attenuation of both increase in neuroinflammatory factory and decrease in trophic factors. This suggests that MC-100093 has the potential to prevent the effects of chronic ethanol consumption involving inflammation and oxidative stress.

Although the subject matter has been described in terms of exemplary embodiments, it is not limited thereto. Rather, the appended claims should be construed broadly, to include other variants and embodiments, which may be made by those skilled in the art.

What is claimed is:

1. A method for treating or preventing alcohol dependence and/or an alcohol associated disease or condition, comprising administrating a pharmaceutical composition comprising an effective amount of a compound having formula (I): 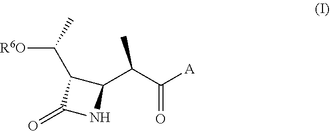
or a hydrates, a solvate, a pharmaceutically acceptable salt, a prodrug, or a complex thereof to a subject in need thereof, wherein:
A is selected from the group consisting of 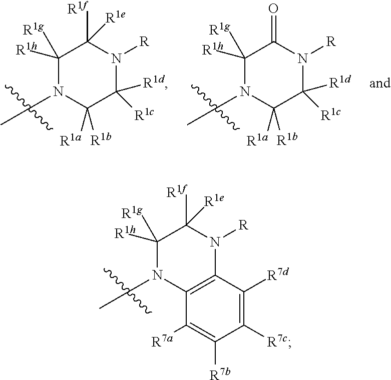
R is selected from the group consisting of hydrogen, C₁₋₆ linear alkyl, C₁₋₆ branched alkyl, optionally substituted aryl, C(O)R², C(O)OR³, C(O)NR^{4a}R^{4b}, SO₂R⁵, and SO₂NH₂;
R^{1a}, R^{1b}, R^{1c}, R^{1d}, R^{1e}, R^{1f}, R^{1g}, and R^{1h} are each independently selected from the group consisting of hydrogen, C₁₋₆ linear alkyl, and C₁₋₆ branch alkyl;
R² is selected from the group consisting of C₁₋₆ linear alkyl, C₁₋₆ branched alkyl, and optionally substituted aryl;
R³ is selected from the group consisting of C₁₋₆ linear alkyl, C₁₋₆ branched alkyl, and optionally substituted aryl;
R^{4a} is selected from the group consisting of C₁₋₆ linear alkyl, C₁₋₆ branched alkyl, and optionally substituted aryl;
R^{4b} is selected from the group consisting of C₁₋₆ linear alkyl, C₁₋₆ branched alkyl, and optionally substituted aryl;
R⁵ is selected from the group consisting of C₁₋₆ linear alkyl, C₁₋₆ branched alkyl, and optionally substituted aryl;
R⁶ is selected from the group consisting of hydrogen, C₁₋₆ linear alkyl, and C(O)R¹;
R^{7a}, R^{7b}, R^{7c}, and R^{7d} are each independently selected from the group consisting of are each independently selected from a group consisting of hydrogen, halogen, OH, C₁₋₆ linear alkyl, C₁₋₆ branched alkyl, C₁₋₆ alkoxy, C₁₋₆ haloalkyl, C₁₋₆ haloalkoxy, cyano, NH(C₁₋₆ alkyl), N(C₁₋₆ alkyl)₂, NHC(O)R⁸, C(O)NHR⁸, C(O)N(R⁸)₂, SH, SC₁₋₆ alkyl, SO₂NH₂, SO₂NHR⁸, SO₂R⁸, and NHSO₂R⁸; and
R⁸ is independently selected at each occurrence from a group consisting of hydrogen, C₁₋₆ linear alkyl, C₁₋₆ branched alkyl, and C₃₋₇ cycloalkyl.

2. The method of claim 1, wherein R^{1b} and R^{1g} are joined together with the atoms to which they are bound to form a ring containing 5, 6, or 7 atoms.

3. The method of claim 1, wherein R^{1b} and R^{1f} are joined together with the atoms to which they are bound to form a ring containing 5, 6, or 7 atoms.

4. The method of claim 1, wherein R^{1d} and R^{1f} are joined together with the atoms to which they are bound to form a ring containing 5, 6, or 7 atoms.

5. The method of claim 1, wherein R^{1b} and R^{1e} are joined together with the atoms to which they are bound to form a ring containing 5 or 6 atoms.

6. The method of claim 1, wherein the compound is selected from the group consisting of:
(3S, 4R)-3-((R)-(1-hydroxy-ethyl)-4-((R)-[1-methyl-2-(4-methyl-piperazin-1-yl)-2-oxo-ethyl]-azetidin-2-one;
tert-butyl 4-((R)-2-((2R,3S)-3-((R)-1-hydroxyethyl)-4-oxoazetidin-2-yl) propanoyl)piperazine-1-carboxylate;
(3S, 4R)-3-((R)-(1-Hydroxy-ethyl)-4-((R)-(1-methyl-2-oxo-2-piperazin-1-yl-ethyl)-azetidin-2-one;
(3S, 4R)-4-((R)-(1-(4-acetylpiperazin-1-yl)-1-oxopropan-2-yl)-3-((R) (1-hydroxyethyl)azetidin-2-one;
(3S,4R)-4-((R)-1-(4-ethylpiperazin-1-yl)-1-oxopropan-2-yl)-3-((R)-1-hydroxyethyl)azetidin-2-one;
(3S,4R)-3-((R)-1-hydroxyethyl)-4-((R)-1-(4-(methylsulfonyl) piperazin-1-yl)-1-oxopropan-2-yl)azetidin-2-one;
(3S,4R)-4-((R)-1-(4-cyclohexylpiperazin-1-yl)-1-oxopropan-2-yl)-3-((R)-1-hydroxyethyl)azetidin-2-one;
(3S,4R)-4-((R)-1-(4-benzoylpiperazin-1-yl)-1-oxopropan-2-yl)-3-((R)-1-hydroxyethyl)azetidin-2-one;
(3S,4R)-3-((R)-1-hydroxyethyl)-4-((R)-1-oxo-1-(4-phenyl piperazin-1-yl)propan-2-yl)azetidin-2-one;
(3S,4R)-3-((R)-1-hydroxyethyl)-4-((R)-1-oxo-1-(4-propyl piperazin-1-yl)propan-2-yl)azetidin-2-one;
(3S,4R)-3-((R)-1-hydroxyethyl)-4-((R)-1-(4-(4-methoxyphenyl) piperazin-1-yl)-1-oxopropan-2-yl)azetidin-2-one;
(3S,4R)-4-((R)-1-(4-(tert-butyl)piperazin-1-yl)-1-oxopropan-2-yl)-3-((R)-1-hydroxyethyl)azetidin-2-one;
4-((R)-2-((2R,3S)-3-((R)-1-hydroxyethyl)-4-oxoazetidin-2-yl)propanoyl)piperazine-1-carboxamide;
(3S,4R)-3-((R)-1-hydroxyethyl)-4-((R)-1-(4-methyl-3,4-dihydro quinoxalin-1(2H)-yl)-1-oxopropan-2-yl)azetidin-2-one;
(R)-1-((2R,3S)-2-((R)-1-(4-methylpiperazin-1-yl)-1-oxopropan-2-yl)-4-oxoazetidin-3-yl)ethyl acetate;
(R)-1-((2R,3S)-2-((R)-1-(4-methylpiperazin-1-yl)-1-oxopropan-2-yl)-4-oxoazetidin-3-yl)ethyl butyrate;
(R)-1-((2R,3S)-2-((R)-1-(4-methylpiperazin-1-yl)-1-oxopropan-2-yl)-4-oxoazetidin-3-yl)ethyl isobutyrate;
(R)-1-((2R,3S)-2-((R)-1-(4-methylpiperazin-1-yl)-1-oxopropan-2-yl)-4-oxoazetidin-3-yl)ethyl pivalate;
and any pharmaceutically acceptable forms thereof.

7. The method of claim 1, wherein the compound is (3S, 4R)-3-((R)-(1-hydroxy-ethyl)-4-((R)-[1-methyl-2-(4-methyl-piperazin-1-yl)-2-oxo-ethyl]-azetidin-2-one, or a pharmaceutically acceptable salt or complex thereof.

8. The method of claim 1, wherein the pharmaceutical composition further comprises at least one excipient.

9. The method of claim 1, wherein the subject is a mammal.

10. The method of claim 1, wherein the subject is a human subject.

11. The method of claim 1, wherein the composition is administrated orally or through injection.

12. The method of claim 1, wherein the effective amount is a dose of the compound in a range of from 0.001 mg/Kg of the subject to 200 mg/Kg of the subject.

13. The method of claim 12, wherein the dose of the compound is in a range of from 1 mg/Kg of the subject to 100 mg/Kg of the subject.

14. The method of claim 1, wherein the method is for treating or preventing alcohol dependence.

15. The method of claim 14, wherein the alcohol dependence is treated or prevented by attenuating alteration in glutamate transporters and/or reducing alcohol associated neuroinflammation.

16. The method of claim 14, wherein the compound is (3S, 4R)-3-((R)-(1-hydroxy-ethyl)-4-((R)-[1-methyl-2-(4-methyl-piperazin-1-yl)-2-oxo-ethyl]-azetidin-2-one, or a pharmaceutically acceptable salt or complex thereof.

17. The method of claim 16, wherein the composition is administrated through injection or orally.

18. The method of claim 1, wherein the method is for attenuating alteration in glutamate transporters in the subject.

19. The method of claim 1, wherein the method is for reducing alcohol associated neuroinflammation in the subject.

20. The method of claim 1, wherein the method is for treating or preventing alcohol associated fatty liver diseases.

21. The method of claim 19, wherein the compound is (3S, 4R)-3-((R)-(1-hydroxy-ethyl)-4-((R)-[1-methyl-2-(4-methyl-piperazin-1-yl)-2-oxo-ethyl]-azetidin-2-one, or a pharmaceutically acceptable salt or complex thereof.

22. The method of claim 21, wherein the composition is administrated through injection or orally.
